SEQUENCE LISTING (44 sequences)

SEQ ID NO: 1 - protein, 189 aa, Homo sapiens [sequence omitted]

SEQ ID NO: 2 - DNA, 570 bp, Homo sapiens
  source 1..570: mol_type = genomic DNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaa

SEQ ID NO: 3 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 4 - DNA, 570 bp, Homo sapiens
  source 1..570: mol_type = genomic DNA
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 5 - protein, 27 aa, Homo sapiens
CTRGHYYDSSGYSLIKGYNYYYYLDVW

SEQ ID NO: 6 - protein, 18 aa, Homo sapiens
CARQGVLGFGVSNWFDPW

SEQ ID NO: 7 - protein, 15 aa, Homo sapiens
CARDLSKLAEAFDIW

SEQ ID NO: 8 - protein, 9 aa, Homo sapiens
CATRTMIVV

SEQ ID NO: 9 - protein, 9 aa, Homo sapiens
LLPTPPDYW

SEQ ID NO: 10 - protein, 19 aa, Homo sapiens
CATEISPAIPPLGYGLGVW

SEQ ID NO: 11 - protein, 9 aa, Homo sapiens
CVRKRVVLL

SEQ ID NO: 12 - protein, 10 aa, Homo sapiens
SGYYWGFDYW

SEQ ID NO: 13 - protein, 20 aa, Homo sapiens
CARMEASYAPTHNFYGLDVW

SEQ ID NO: 14 - protein, 12 aa, Homo sapiens
CARDQGGPFDHW

SEQ ID NO: 15 - protein, 16 aa, Homo sapiens
CTLDYFGSGSNYNKYW

SEQ ID NO: 16 - protein, 20 aa, Homo sapiens
CATLPDDYGVDYGYWYFDLW

SEQ ID NO: 17 - protein, 5 aa, Homo sapiens
CARGL

SEQ ID NO: 18 - protein, 9 aa, Homo sapiens
MRCNWFDPW

SEQ ID NO: 19 - protein, 30 aa, Homo sapiens
CARGVRITIFGVAGGWTSPEDGEKDGLDPW

SEQ ID NO: 20 - protein, 21 aa, Homo sapiens
CARHRLATYYYESSGYYFDYW

SEQ ID NO: 21 - protein, 18 aa, Homo sapiens
CAGWGRTLPFLSNWFDPW

SEQ ID NO: 22 - protein, 17 aa, Homo sapiens
CARDWDMATIRGGGDYW

SEQ ID NO: 23 - protein, 16 aa, Homo sapiens
CTTQIYCTNGVCADYW

SEQ ID NO: 24 - protein, 14 aa, Homo sapiens
CVLPLVGTVYVGYW

SEQ ID NO: 25 - protein, 17 aa, Homo sapiens
CARDWNYGTNSFWFDPW

SEQ ID NO: 26 - protein, 16 aa, Homo sapiens
CARIRDYYASGAHDFW

SEQ ID NO: 27 - protein, 24 aa, Homo sapiens
CARFGGRDFWSGYSGYYHYGMDVW

SEQ ID NO: 28 - protein, 21 aa, Homo sapiens
CVHRQGRTLLRGAMSPYFDFW

SEQ ID NO: 29 - protein, 16 aa, Homo sapiens
CVKGDYGRNPGHFEYW

SEQ ID NO: 30 - protein, 7 aa, Homo sapiens
CARDCDS

SEQ ID NO: 31 - protein, 5 aa, Homo sapiens
GVTTL

SEQ ID NO: 32 - protein, 4 aa, Homo sapiens
FDYW

SEQ ID NO: 33 - protein, 20 aa, Homo sapiens
CARILSDFGDHDRRDAFDVW

SEQ ID NO: 34 - protein, 13 aa, Homo sapiens
CASEIVGWAFDYW

SEQ ID NO: 35 - protein, 12 aa, Homo sapiens
CATSALGQVDNW

SEQ ID NO: 36 - protein, 22 aa, Homo sapiens
CSRDGLGIVATGDVDSGGLDRW

SEQ ID NO: 37 - protein, 23 aa, Homo sapiens
CVKDLSNGYYSLDANHYFGMDVW

SEQ ID NO: 38 - protein, 21 aa, Homo sapiens
CVRGEDEVTAIDYYYFGMDVW

SEQ ID NO: 39 - protein, 17 aa, Homo sapiens
CAREGDDYDDYNYLDYW

SEQ ID NO: 40 - protein, 13 aa, Homo sapiens
CARRFGGSYFDYW

SEQ ID NO: 41 - protein, 17 aa, Homo sapiens
CAKGIYSSSFTRARDSW

SEQ ID NO: 42 - protein, 18 aa, Homo sapiens
CARGLSWEVPAAAWFDPW

SEQ ID NO: 43 - protein, 17 aa, Homo sapiens
CARSYSSSLRGDWFDPW

SEQ ID NO: 44 - protein, 9 aa, synthetic construct
LLPTPPDYW

(19) United States
(12) Patent Application Publication
(10) Pub. No.: US 2026/0327717 A1
(43) Pub. Date: Oct. 22, 2026

(54) METHODS FOR CHARACTERIZATION OF CIRCULATING TUMOR CELLS

(71) Applicants: DANA-FARBER CANCER INSTITUTE, INC., Boston, MA (US); THE GENERAL HOSPITAL CORPORATION, Boston, MA (US)

(72) Inventors: Ankit K. Dutta, Boston, MA (US); Jean-Baptiste Alberge, Vanves (FR); Irene Ghobrial, Boston, MA (US); Gad Getz, Belmont, MA (US)

(21) Appl. No.: 19/679,336

(22) Filed: May 15, 2026

Related U.S. Application Data
(63) Continuation of application No. 18/715,622, filed on Dec. 2, 2022.
(60) Provisional application No. 63/285,875, filed on Dec. 3, 2021.

(51) Int. Cl. C12Q 1/6886; G01N 33/575

(52) U.S. Cl. CPC C12Q 1/6886; G01N 33/5758; C12Q 2600/106; C12Q 2600/156; G01N 2333/70503; G01N 2333/70589; G01N 2333/70596

(57) ABSTRACT

The invention features methods for the identification of genomic aberrations in circulating tumor cells isolated from peripheral blood. In embodiments, the methods involve isolation of a small number of purified circulating multiple myeloma cells, purification of genomic DNA from the cells, and sequencing of the genomic DNA.

### CROSS-REFERENCE TO RELATED APPLICATION

This application is a continuation of U.S. patent application Ser. No. 18/715,622, filed May 31, 2024, which is the § 371 U.S. National Stage of International Application No. PCT/US2022/080796, filed Dec. 2, 2022, which claims the benefit of U.S. Provisional Application No. 63/285,875, filed Dec. 3, 2021, the disclosures of which are incorporated by reference herein in their entireties.

### SEQUENCE LISTING

This application contains a Sequence Listing electronically submitted via Patent Center to the United States Patent and Trademark Office as an XML file entitled “680_3089US01.xml” having a size of 40,585 bytes and created on May 31, 2024. The information contained in the Sequence Listing is incorporated by reference herein.

### BACKGROUND OF THE INVENTION

Multiple Myeloma (MM) remains an incurable hematologic malignancy characterized by the aberrant proliferation of clonal plasma cells (PCs) within the bone marrow (BM), which, in many cases, develops from the asymptomatic disease stages known as Monoclonal Gammopathy of Undetermined Significance (MGUS) and Smoldering Multiple Myeloma (SMM). Despite the development of effective new therapies, which have led to improved outcomes, most MM patients inevitably relapse and require further treatment. Diagnosis of precursor disease remains an incidental process with no routine screening or monitoring tests available. This highlights the need for advancement in early detection methods for precursor patients and resulting precision prevention and intervention to delay or intercept disease progression.

Methods for detection of multiple myeloma typically involve a bone biopsy, which is considered the gold standard for diagnosis and monitoring of MM progression. However, bone biopsy is an intrusive and painful procedure with possible secondary complications for patients. There is an urgent need for improvement in robust early detection methods that are biology based, able to capture the full picture of temporal and spatial nature of disease, and that are ideally minimally invasive for patients.

### SUMMARY OF THE INVENTION

As described below, the present invention features compositions and methods for minimally invasive characterizing a plasma cell dyscrasia (e.g., monoclonal gammopathy of undetermined significance, smoldering multiple myeloma, multiple myeloma) in a biological sample from a subject.

In one aspect, the invention features a method of characterizing a hematological malignancy in a subject. The method involves determining the number of circulating tumor cells (CTCs) in peripheral blood of a subject. The number of circulating multiple myeloma cells is indicative of disease stage.

In another aspect, the invention features a method of measuring a multiple myeloma 2/20/20 risk group in a subject with smoldering multiple myeloma. The method involves counting circulating tumor cells (CTCs) in a liquid biopsy collected from the subject. An elevated count of CTCs relative to a reference identifies the subject as having elevated risk for development of active multiple myeloma (MM).

In any of the above aspects, or embodiments thereof, a higher count of CTCs is indicative of a more advanced disease stage.

In any of the above aspects, or embodiments thereof, the number of circulating multiple myeloma cells is indicative of 2/20/20 risk stage. In any of the above aspects, or embodiments thereof, a higher count of CTCs is indicative of a higher 2/20/20 risk stage. In any of the above aspects, or embodiments thereof, a CTC count of less than about 3 is indicative of a low 2/20/20 risk stage. In any of the above aspects, or embodiments thereof, a CTC count of between about 5 and 24 is indicative of intermediate 2/20/20 risk stage. In any of the above aspects, or embodiments thereof, a CTC count of greater than about 170 is indicative of high 2/20/20 risk stage. In any of the above aspects, or embodiments thereof, a CTC count of greater than about 100, 200, 300, 400, 500, 600, 700, 800, 900, or 1000 is indicative that the subject has multiple myeloma. In any of the above aspects, or embodiments thereof, a CTC count of greater than about 1000 is indicative that the subject has multiple myeloma.

In any of the above aspects, or embodiments thereof, the CTCs are counted using an immunofluorescence-based technique. In any of the above aspects, or embodiments thereof, the immunofluorescence-based technique is fluorescence activated cell sorting (FACS). In any of the above aspects, or embodiments thereof, the subject is identified as having elevated risk if the CTC count is greater than about 20 per 4 ml of liquid biopsy.

In another aspect, the invention features a method of characterizing a hematological malignancy in a subject. The method involves isolating circulating tumor cells (CTCs) from peripheral blood of a subject, and detecting an alteration in the genome of the isolated cells.

In another aspect, the invention features a method of selecting a subject for treatment of a hematological malignancy, the method involving administering to the subject in need thereof an agent for the treatment of a hematological malignancy, where the subject is selected by isolating circulating tumor cells from peripheral blood of a subject, and detecting an alteration in the genome of the isolated cells that identifies the subject as having a hematological malignancy selected from one or more of MM, MGUS, and SMM.

In another aspect, the invention features a method of monitoring progression of a hematological malignancy in a subject. The method involves periodically isolating circulating tumor cells from peripheral blood of a subject, and detecting an alteration in the genome of the isolated cells, where an increase in the presence of alterations in the cells identifies the hematological malignancy as having progressed to a more advanced stage.

In any of the above aspects, or embodiments thereof, the circulating tumor cells have an immunophenotype of CD138⁺CD38⁺CD45⁻CD19⁻.

In any of the above aspects, or embodiments thereof, the alteration is characterized using sequencing and/or polynucleotide probe hybridization. In embodiments, the sequencing is carried out on DNA isolated from the circulating tumor cells. In embodiments, the sequencing is whole genome sequencing. In any of the above aspects, or embodiments thereof, the method does not involve any whole-genome amplification step. In embodiments, the amount of DNA sequenced is less than about 1000 pg. In embodiments, the amount of DNA sequenced is less than about 100, 10000, 100000, or 1000000 pg. In embodiments, the amount of DNA sequenced is less than about 100 pg. In embodiments, the sequence coverage is between 0.01× and 100×. In embodiments, the sequence coverage is at least about 120×, 150×, 200×, 300×, 400×, 500×, 600×, 700×, 800×, 900×, 1000×, or greater. In any of the above aspects, or embodiments thereof, the whole genome sequencing detects a genomic event selected from one or more of an aneuploidy, a translocation, a chromosomal gain, a chromosomal deletion, and a driver mutation. In embodiments, the aneuploidy is a hyperploidy and/or a monoploidy. In embodiments, the hyperploidy is a trisomy. In embodiments, the hyperploidy is a tetrasomy. In embodiments, the translocation is selected from one or more of t(11;14), t(14;16), t(4;14), t(6;14), t(14;20), t(8;14), t(2;8), and t(8;22). In embodiments, the chromosomal gain is a 1q gain. In embodiments, the chromosomal deletion is a 1p deletion, a chromosome 13q deletion, a chromosome 16q deletion, or a chromosome 17p deletion. In embodiments, the driver mutation contains a mutation to a gene associated with the Ras/Raf/MAPK pathway. In embodiments, the driver mutation contains a non-silent mutation to a KRAS and/or an NRAS gene sequence. In embodiments, the driver mutation is associated with an alteration to an A146, G12, G13, Q61, or K117 amino acid of a KRAS and/or NRAS polypeptide. In embodiments, the driver mutation contains a mutation to a DIS3, FAM46C, BRAF, or TP53 gene sequence.

In any of the above aspects, or embodiments thereof, DNA is isolated from the circulating tumor cells. In embodiments, the DNA is isolated from at least about 10 or 100 circulating tumor cells. In any of the above aspects, or embodiments thereof, the circulating tumor cells contain a tumor cell fraction of at least about 10%. In any of the above aspects, or embodiments thereof, the circulating tumor cells contain a tumor cell fraction of at least about 30%.

In any of the above aspects, or embodiments thereof, the subject has monoclonal gammopathy of undetermined significance (MGUS), smoldering multiple myeloma (SMM), or multiple myeloma.

In any of the above aspects, or embodiments thereof, the circulating tumor cells are isolated from the peripheral blood using an immunofluorescence-based technique. In embodiments, the immunofluorescence-based technique is fluorescence activated cell sorting (FACS). In any of the above aspects, or embodiments thereof, the circulating tumor cells are sorted based upon the immunophenotype CD138+ and/or CD38+. In any of the above aspects, or embodiments thereof, the circulating tumor cells are sorted based upon the immunophenotype CD19− and/or CD45−.

In any of the above aspects, or embodiments thereof, the subject is a mammal. In any of the above aspects, or embodiments thereof, the subject is a human.

In any of the above aspects, or embodiments thereof, the agent is a chemotherapeutic agent.

In any of the above aspects, or embodiments thereof, the method further involves administering to the subject a tandem autologous stem cell transplant, and the alteration is selected from one or more of t(4;14), t(14;16), t(14;20), or del(17p). In any of the above aspects, or embodiments thereof, the agent contains venetoclax, and the alteration is t(11;14).

In any of the above aspects, or embodiments thereof, the method further involves determining the 2/20/20 risk group of the subject based upon the detection of the alteration. In embodiments, a high-risk group selects the subject for treatment with a tandem transplant, immunotherapy, or consolidation therapy.

In any of the above aspects, or embodiments thereof, the hematological malignancy is SMM or MGUS.

The invention provides methods for identification of genomic aberrations in circulating tumor cells (e.g., circulating multiple myeloma cells (CMMCs)) isolated from a liquid biopsy. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Kirsten rat sarcoma virus (KRAS) polypeptide” is meant a KRAS protein or fragment thereof, having GTPase activity and having at least about 85% amino acid sequence identity to GenBank Accession No. AAB59445.1. An exemplary KRAS amino acid sequence from Homo sapiens is provided below GenBank: AAB59445.1):

MTEYKLVVVGAGGVGKSALTIQLIQNHFVDEYDPTIEDSYRKOVVIDGETCLLDILDTAGQEEY
SAMRDQYMRTGEGFLCVFAINNTKSFEDIHHYREQIKRVKDSEDVPMVLVGNKCDLPSRTVDTK
QAQDLARSYGIPFIETSAKTRORVEDAFYTLVREIRQYRLKKISKEEKTPGCVKIKKCIIM.

By “Kirsten rat sarcoma virus (KRAS) polynucleotide” is meant a nucleic acid molecule encoding a KRAS polypeptide, as well as the introns, exons, and regulatory sequences associated with its expression, or fragments thereof. In embodiments, a KRAS polynucleotide is the genomic sequence, mRNA, or gene associated with and/or required for KRAS expression. An exemplary KRAS nucleotide sequence from Homo sapiens is provided below (GenBank: AH005283.2):

ATGACTGAATATAAACTTGTGGTAGTTGGAGCTGGTGGCGTAGGCAAGAGTGCCTTGACGATAC
AGCTAATTCAGAATCATTTTGTGGACGAATATGATCCAACAATAGAGGATTCCTACAGGAAGCA
AGTAGTAATTGATGGAGAAACCTGTCTCTTGGATATTCTCGACACAGCAGGTCAAGAGGAGTAC
AGTGCAATGAGGGACCAGTACATGAGGACTGGGGAGGGCTTTCTTTGTGTATTTGCCATAAATA
ATACTAAATCATTTGAAGATATTCACCATTATAGAGAACAAATTAAAAGAGTTAAGGACTCTGA
AGATGTACCTATGGTCCTAGTAGGAAATAAATGTGATTTGCCTTCTAGAACAGTAGACACAAAA
CAGGCTCAGGACTTAGCAAGAAGTTATGGAATTCCTTTTATTGAAACATCAGCAAAGACAAGAC
AGAGAGTGGAGGATGCTTTTTATACATTGGTGAGAGAGATCCGACAATACAGATTGAAAAAAAT
CAGCAAAGAAGAAAAGACTCCTGGCTGTGTGAAAATTAAAAAATGCATTATAATGTAA.

By “Neuroblastoma RAS viral oncogene homolog (NRAS) polypeptide” is meant an NRAS protein or fragment thereof, having GTPase activity and having at least about 85% amino acid sequence identity to GenBank Accession No. CAA26529 An exemplary NRAS amino acid sequence from Homo sapiens is provided below (GenBank: CAA26529):

MTEYKLVVVGAGGVGKSALTIQLIQNHFVDEYDPTIEDSYRKOVVIDGETCLLDILDTAGQEEY
SAMRDQYMRTGEGFLCVFAINNSKSFADINLYREQIKRVKDSDDVPMVLVGNKCDLPTRTVDTK
QAHELAKSYGIPFIETSAKTRQGVEDAFYTLVREIRQYRMKKLNSSDDGTQGCMGLPCVVM.

By “Neuroblastoma RAS viral oncogene homolog (NRAS) polynucleotide” is meant a nucleic acid molecule encoding an NRAS polypeptide, as well as the introns, exons, and regulatory sequences associated with its expression, or fragments thereof. In embodiments, an NRAS polynucleotide is the genomic sequence, mRNA, or gene associated with and/or required for NRAS expression. An exemplary NRAS nucleotide sequence from Homo sapiens is provided below (GenBank: X02751.1):

ATGACTGAGTACAAACTGGTGGTGGTTGGAGCAGGTGGTGTTGGGAAAAGCGCACTGACAATCC
AGCTAATCCAGAACCACTTTGTAGATGAATATGATCCCACCATAGAGGATTCTTACAGAAAACA
AGTGGTTATAGATGGTGAAACCTGTTTGTTGGACATACTGGATACAGCTGGACAAGAAGAGTAC
AGTGCCATGAGAGACCAATACATGAGGACAGGCGAAGGCTTCCTCTGTGTATTTGCCATCAATA
ATAGCAAGTCATTTGCGGATATTAACCTCTACAGGGAGCAGATTAAGCGAGTAAAAGACTCGGA
TGATGTACCTATGGTGCTAGTGGGAAACAAGTGTGATTTGCCAACAAGGACAGTTGATACAAAA
CAAGCCCACGAACTGGCCAAGAGTTACGGGATTCCATTCATTGAAACCTCAGCCAAGACCAGAC
AGGGTGTTGAAGATGCTTTTTACACACTGGTAAGAGAAATACGCCAGTACCGAATGAAAAAACT
CAACAGCAGTGATGATGGGACTCAGGGTTGTATGGGATTGCCATGTGTGGTGATGTAA.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “aneuploidy” is meant in the context of a cell having an abnormal number of chromosomes relative to a cell of normal ploidy.

By “alteration” is meant a change in the structure, expression levels or activity of a gene or polypeptide as detected by standard art known methods such as those described herein. The alteration can be an increase or a decrease. As used herein, an alteration includes a 10% change in expression levels, preferably a 25% change, more preferably a 40% change, and most preferably a 50% or greater change in expression levels. In some embodiments, the alteration is a change in the sequence of the genome of a cell. Exemplary sequence changes include, but are not limited to deletions, hyperdiploidy, copy number abnormalities, and translocations.

The term “amplification” means any method employing a primer and a polymerase capable of replicating a target sequence with reasonable fidelity. In embodiments the target sequence is a genome and the amplification is “whole-genome amplification.” In some instances a method of the invention may comprise an amplification step (e.g., a PCR step for adapter ligation and/or final library amplification) but specifically exclude any whole-genome amplification step, which is typically a preceding step when sequencing samples containing low levels of DNA (e.g., low numbers of cells). In some embodiments, the methods of the invention do not include any amplification of a DNA sample (e.g., whole genome amplification) prior to library preparation. Amplification may be carried out by natural or recombinant DNA polymerases such as TaqGold™, T7 DNA polymerase, Klenow fragment of E. coli DNA polymerase, and reverse transcriptase. Non-limiting examples of amplification methods include PCR and/or whole genome amplification. Amplification may involve thermocycling or isothermal amplification (such as through the methods RPA or LAMP).

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

As used herein, the term “coverage” refers to the percentage of genome covered by reads. In one embodiment, low coverage or ultra low pass coverage is less than about 1×. Coverage also refers to, in shotgun sequencing, the average number of reads representing a given nucleotide in the reconstructed sequence. It can be calculated from the length of the original genome (G), the number of reads (N), and the average read length (L) as N*(L/G). Biases in sample preparation, sequencing, and genomic alignment and assembly can result in regions of the genome that lack coverage (that is, gaps) and in regions with much higher coverage than theoretically expected. It is important to assess the uniformity of coverage, and thus data quality, by calculating the variance in sequencing depth across the genome. The term depth may also be used to describe how much of the complexity in a sequencing library has been sampled. All sequencing libraries contain finite pools of distinct DNA fragments. In a sequencing experiment only some of these fragments are sampled.

“Detect” refers to identifying the presence, absence or amount of the analyte to be detected.

By “disease” is meant any condition or disorder that damages or interferes with the normal function of a cell, tissue, or organ. In embodiments, the disease is any disease that may be characterized by isolating circulating tumor cells (CTCs). In some instances, the disease is a hematological malignancy. Examples of diseases include plasma cell dyscrasias (e.g., a monoclonal gammopathy), such as monoclonal gammopathy of undermined significance (MGUS), smoldering multiple myeloma (SMM), symptomatic multiple myeloma, Waldenstrom macroglobulinemia (WM), amyloidosis (AL), plasmacytoma syndrome (e.g., solitary plasmacytoma of bone, extramedullary plasmacytoma), light chain deposition disease, and heavy-chain disease. Further non-limiting examples of diseases include any B cell or plasma cell neoplasm.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increase” is meant to alter positively by at least 5% relative to a reference. An increase may be by 5%, 10%, 25%, 30%, 50%, 75%, or even by 100%.

The terms “isolated,” “purified,” or “biologically pure” refer to material that is free to varying degrees from components which normally accompany it as found in its native state. “Isolate” denotes a degree of separation from original source or surroundings (e.g., non circulating tumor cells and/or peripheral blood mononuclear cells). “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. An isolated cell(s) are free from other cells not of interest to an analysis (e.g., free of non-plasma cells and/or free of non-circulating multiple myeloma cells).

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein, cell, or polynucleotide having an alteration in expression level, genome sequence, or activity that is associated with a condition, disease, or disorder. Non-limiting examples of markers include circulating tumor cells, such as circulating multiple myeloma cells (CMMCs). In some embodiments, a marker can include a genomic event, such as aneuploidy (e.g., hyperploidies, such as trisomies, or tetrasomies; and monoploidies), translocations (e.g., t(4;14), t(6;14), t(8;14), t(11;14), t(14;16), t(14;20), t(2;8), and t(8;22)), chromosomal arm gains or deletions (e.g., chromosome 1q gain, chromosome 1p deletion, chromosome 13q deletion, chromosome 16q deletion, and chromosome 17p deletion), and/or driver mutations (e.g., mutations in the Ras/Raf/MAPK pathway (e.g., mutations to KRAS or NRAS), and/or mutations to DIS3, FAM46C, BRAF, and/or TP53). The event detected can be predictive of risk and MM progression. In embodiments, a driver mutation is selected from any non-silent mutation (e.g., a G12, G13, Q61, K117, or A146 alteration) to KRAS or NRAS. In some instances, a driver mutation is selected from any non-silent mutation to any one or more of the following genes: KRAS, NRAS, DIS3, BRAF, FAM46C, TP53, MYC, MAX, IGLL5, TRAF3, DUSP2, TCL1A, TRAF2, CYLD, LTB, HIST1H1E, BCL7A, SP140, NFKBIA, EGR1, PABPC1, PRKD2, TBC1D29, IRF4, RB1, TGDS, PTPN11 FUBP1, RPL5, FGFR3, SAMHD1, ACTG1, HIST1H1B, NFKB2, KMT2B, KLHL6, RASA2, PIM1, PRDM1, DTX SETD2, BHLHE41, RPL10, BTG1, RPS3A, CCND1, RPRD1B, HIST1H1D, ZNF292, RFTN1, CDKN1B, LCE1D, XBP1, IRF1, POT1, HIST1H2BK, ABCF1, ZFP36L1, TET2, ARID2, KDM6A, EP300, ARID1A, NCOR1, HUWE1, CDKN2C, SF3B1, ATM, NF1, CREBBP, DNMT3A, MAFB, MAF, KDM5C, UBR5, PIK3CA, IDH1, MCL1, BIRC2, MLL1, MLL2, MAML2, MAN2C1, IDH2, KMT2C, and ATRX.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively by at least 5% relative to a reference. A reduction may be by 5%, 10%, 25%, 30%, 50%, 75%, or even by 100%.

By “reference” is meant a standard or control condition. In embodiments, a reference is a healthy subject or cell or the genome sequence of a healthy subject or cell.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. A reference sequence can be the genome of a healthy cell or a portion thereof. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “remission” is meant a subject having substantially no signs or symptoms of multiple myeloma. In embodiments, a multiple myeloma subject in remission shows little-to-no signs or symptoms of multiple myeloma and/or shows signs or symptoms of multiple myeloma similar to those observed in a healthy subject and/or a subject having a non-active multiple myeloma (e.g., MGUS or SMM).

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “venetoclax” is meant a compound with the chemical structure

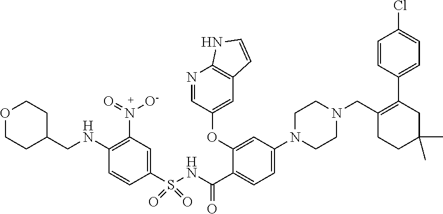

and corresponding to CAS No. 1257044-40-8, or pharmaceutically acceptable salts thereof. Venetoclax is a BH3-mimetic.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1E provides overviews, graphs, and plots showing that detection and enumeration of circulating tumor cells (CTCs) correlates with multiple myeloma (MM) disease pathology. FIG. 1A provides an overview of the experimental workflow used to enumerate CTCs (n=261) and characterize their genomic abnormalities (n=51). FIG. 1B provides a distribution of CTC counts across precursor disease stages of MM. Text indicates proportion of participants with no CTCs detected. FIG. 1C provides a boxplot and density graphs of CTCs enumerated between disease precursor stages MGUS and SMM. FIG. 1D provides a boxplot and density graphs showing CTC counts associated with 20/2/20 SMM risk classification when available. FIG. 1E provides Kaplan-Meier curves depicting probability of progression from SMM to overt myeloma or death based on CTC enumeration. *: p<0.05, **:p<0.01, ***:p<0.001, ****:p<0.0001.

FIGS. 2A-2D provides plots and heatmaps showing that MinimuMM-seq reveals CTCs reflect the major BM clone and could replace molecular cytogenetics. FIG. 2A provides a boxplot of the number of cells sequenced for BMPCs (left) versus CTCs (right). In 8 cases selected CD138+ BMPC bulk genomic DNA was used. FIG. 2B provides a plot showing tumor purity in BMPCs and CTCs assessed by the ABSOLUTE algorithm and based on copy number abnormalities, and single nucleotide variant (SNVs) multiplicity and fraction of alternate reads. FIG. 2C provides plots showing categorical classification of successful Fluorescence In Situ Hybridization (FISH) probes by their presence or absence in the whole genome sequencing (WGS) data of BMPCs and CTCs. FISH failures and untested FISH probes are excluded from this graph. FIG. 2D provides a genome wide copy number abnormalities heatmap and translocation discovery (right), in participants with matched CTC and BM samples (n=24), with comparison to clinical FISH report results. Each row is split into a top panel dedicated to CTCs profiling and a lower panel for BMPCs. Key differences between compartments are highlighted by an arrow or dark gray text in the table. FISH failures/within normal limits are represented in light gray text in the table. Ns: non-significant, *: p<0.05, **: p<0.01, ***:p<0.001.

FIGS. 3A-3D provide heatmaps, plots, and examples showing that MinimuMM-seq enables genomic profiling of CTCs for unbiased WGS based molecular analyses. FIG. 3A provides a copy number heatmap and translocation matrix from the discovery cohort of CTCs only sequencing (n=27). FIG. 3B provides plots showing for patients with longitudinal follow-up, concordance of previous FISH reports with WGS results. FISH failures and untested FISH probes are excluded from this graph. FIGS. 3C-3D provide two examples of clinical relevance depicting mutations detected by MinimuMM-seq on CTCs. Both participants are clinically diagnosed as SMM, and an assay characterizes novel translocation (CTF025: IGH-MYC), gains and losses of chromosomes (CTF025: trisomies), as well as driver point mutations (CTF031: TP53 resulting in bi-allelic hit; CTF025: NRAS) using CTCs which may refine patient tumor classification using blood-based sampling only.

FIGS. 4A-4C provide graphs and reconstructions showing that serial WGS of CTCs reveals disease clonal architecture and evolutionary history. FIG. 4A (i) shows a comparison of mutation density occurring between bone marrow plasma cells (BMPCs, x-axis) and peripheral blood (CTCs, y-axis) compartments in SMM participant CTF013 estimated by ABSOLUTE. Recurrent mutations in MM are annotated. FIG. 4A(ii) shows a phylogenetic reconstruction of tumor architecture between BMPCs and CTCs of participant CTF013 after mutation clustering with PhylogicNDT shows preferential circulation of subclones. FIGS. 4B and 4C (i) show comparisons of mutational density from longitudinal sampling of CTCs in SMM. In example of participant CTF004 (B), serial blood was sampled at initial screening (x-axis) and at follow-up 2.3 years later (y-axis). For participant CTF032 (C), serial blood was sampled before (x-axis) and 4 months after start of treatment (y-axis). FIGS. 4B and 4C (ii) show longitudinal reconstructions of CTC clonal evolution for participants CTF004 (B), and CTF032 (C). CCF: Cancer cell fraction.

FIGS. 5A-5B provide matrix plots showing that genomic profiling of CTCs can be used to replace bone marrow FISH for risk classification of patients. Matrix plots highlighting the diagnostic yield of MinimuMM-seq for 51 patients across the disease stages of MGUS, SMM and MM. Each row is a participant, and each column is a genomic abnormality with potential clinical significance in MM. FIG. 5A provides a matrix plot showing in 24 patients that had matched CTCs at the time of BM, head-to-head results detected by both FISH and WGS are highlighted in black, while additional yield detected by WGS is represented in dark gray. The bottom rows show risk stratification enabled by results of CTC sequencing compared to standard clinical FISH. FIG. 5B provides a matrix plot showing abnormality detection and risk classification in the CTC-only cohort when only peripheral blood was sampled from 27 patients.

FIGS. 6A-6E provide plots and examples showing that CTC sequencing enables cohort-level genomic study of mutational signatures and complex structural events. FIG. 6A provides an estimation of weight of mutational processes in the CTC, sorted by known mutational signatures and represented in absolute (top) and 1-normalized values (bottom). Asterisks represent MAF-rearranged tumors. Plus (+) sign represents truncation for participant CTF058 (APOBEC Weight 30,828±71). SBS: single-base substitution. FIGS. 6B-E provide examples showing characterization of complex structural variants from whole-genome sequencing data. FIGS. 6B-C provide examples showing CTF033 chromothripsis of the long arm of chromosome 3 at the genome scale (B) and zoomed on chromosome 3 only (C). FIGS. 6D-E provide examples showing chromoplexy of chromosomes 7, 8, and 18 involving MYC on chromosome 8 in matched bone marrow (D) and peripheral blood (E) from CTF034.

FIGS. 7A-7E provide plots and graphs showing the correlation between clinical measures of disease pathology, survival, and circulating tumor cells enumeration. FIG. 7A provides a boxplot of CTCs enumerated between disease precursor stages MGUS and SMM, and participants with overt disease (MM). FIGS. 7B-C provide comparison and correlation of enumeration results from circulating tumor cells (CTCs) of multiple myeloma precursor patients with (B) plasma cells involvement in the bone marrow given as BMPC percentage (N=92 with successful count), and (C) M-spike protein concentration (N=85 with successful quantification). FIGS. 7D-E provide Kaplan-Meier curves depicting progression-free survival for smoldering multiple myeloma patients (N=109) based on CTC count quartiles with overall p-value (D) and quartiles Q1 vs Q2, Q3, and Q4 (E).

FIGS. 8A-8C provide images, plots, and graphs showing isolation of pure circulating tumor cells from peripheral blood of precursor disease patients. FIG. 8A provides images showing that enriched CTCs are intact cells with immunophenotype of PCs (138+38+45−). FIG. 8B provides plots showing that copy number profiling of MM cells by ultra-low pass whole-genome sequencing in the bone marrow (top) and in matched circulating tumor cells (bottom) illustrates tumor origin and genomic abnormalities. In this example SMM patient concordance of deletion 13, 16 and 22 is observed in both samples. FIG. 8C provides a graph showing detection of IGH translocations per number of cells sequenced and sequence genome coverage. Dashed line at 50 CTCs. Genome coverage is indicated as mean (X) with 95% central interval.

FIGS. 9A-9E provide readouts, plots, and graphs showing that CTC sequencing enables cohort-level genomic characterization of tumor in MM precursor stages FIG. 9A provides a graph showing effective sequence coverage calculated post-alignment with 95% intervals by number of cells captured. FIG. 9B provides a plot showing the estimated power to detect single-nucleotide variants (SNVs) given actual sequence coverage and tumor purity achieved in the context of a clonal mutation (left, cancer cell fraction CCF 100%) and of a subclonal mutation (right, CCF=50% shown). FIG. 9C provides a graph showing the power of SNV detection by number of cells sequenced given purity, ploidy, and genomic coverage in a scenario of a 100% cancer cell fraction (CCF), in plain circles, and of a 50% CCF in triangles. FIG. 9D provides For patients underpowered (left, N=2), and powered at 80% to detect SNVs (right, N=15/17), bar plot representation of total number of mutations detected in each compartment (BMPCs and CTCs) by an assay with CCF estimate >33%, and intersection between both compartments (black dots). Dashed lines represent reference range obtained from the Oben et al. study. FIG. 9E provides a readout showing double-hit hotspot mutation at KRAS p.G12 and p.G13 in matched bone marrow and peripheral blood from participant CTF013.

FIGS. 10A-10E provide heatmaps and graphs showing longitudinal and tissue-matched genomic characterization of driver mutations. FIG. 10A provides a genome-wide copy number abnormalities heatmap (left), and translocation discovery (right), with comparison to clinical FISH reports. Each row is split into a top panel dedicated to CTCs profiling at initial screening date (T0), and subsequent follow-up screenings below (T1, T2). FIG. 10B provides a graph of cancer cell fraction of non-silent mutations in myeloma driver genes at T0 (light gray) and T1 (dark gray). Asterisks represent mutations discovered only at the highlighted timepoint, but CCF is given in validation (force-calling) mode. FIG. 10C provides graphs showing that categorical classification of mutations in known commonly mutated genes detected in this cohort. Top BMPC and CTC rows represent de novo detection of mutation, while bottom validated (Valid.) BMPC and CTC rows represent cross-compartment validation of mutation by force-calling. Known hotspots (G12, G13, Q61 from RAS mutants) are shown in red. FIG. 10D provides a graph showing the fraction of participants with 6 common myeloma driver genes with one or more mutations detected in a known hotspot or outside of a known hotspot. FIG. 10E provides Cancer cell fraction of non-silent mutations in known commonly mutated genes of myeloma between matched CTCs (dark gray) and BMPCs (light gray). Similar to panel B, asterisks represent mutations discovered only at the highlighted timepoint, but CCF is given in validation (force-calling) mode. Quotation marks represent splice site variants.

FIGS. 11A-11B provides graphs showing comparisons of mutational processes between BMPCs and CTCs assigned to most likely PCAWG composite reference signature. FIG. 11A provides a graph showing cosine similarity between BMPCs and CMMCs for each participant with matched samples using raw mutational data and bootstrapping, Mean and 95% confidence intervals are shown. FIG. 11B provides bar graphs representing signature weight (left) and normalized weight (right) in BMPCs and CTCs, per each matched sample. Mean is aggregated from all NMF runs. Plus (+) sign symbolizes truncation for CTF058 (APOBEC Weight, CTCs: 30,828±71, BMPCs: 31,455±91).

### DETAILED DESCRIPTION OF THE INVENTION

The invention features compositions and methods for non-invasively characterizing a monoclonal gammopathy (e.g., monoclonal gammopathy of undetermined significance, smoldering multiple myeloma, multiple myeloma) in a biological sample from a subject. In embodiments, the methods of the invention do not include a whole-genome amplification step.

The invention is based, at least in part, upon the discovery, that monoclonal gammopathies can be non-invasively characterized by (i) detecting alterations in the number of multiple myeloma cells present in the peripheral blood of a subject; or (ii) by isolating multiple myeloma cells from the peripheral blood of a subject, and characterizing such cells (e.g., whole genome sequencing). Such characterization may involve, for example, the sequencing of polynucleotides (e.g., unamplified DNA, such as genomic DNA) from circulating multiple myeloma cells to detect genetic abnormalities (e.g., translocations or hyperploidy) associated with a multiple myeloma. In the Examples provided below, circulating tumor cells were isolated from peripheral blood of patients with multiple myeloma, smoldering multiple myeloma (SMM), or monoclonal gammopathy of undetermined significance (MGUS). Advantageously, the methods of the invention do not require prior knowledge of patient abnormalities to target.

The invention is also based, at least in part, upon the development of a new approach called “MinimuMM-seq” (Minimally Invasive Multiple Myeloma sequencing) that enabled detection of translocations and copy number abnormalities through whole-genome sequencing of highly pure CTCs. The approach leveraged advancements in tumor cell enrichment strategies, low input library construction, and tailored cancer genomics analyses to enable WGS of CTCs for systematic genomic profiling of pathognomonic MM events. Application of the approach to CTCs in a cohort of 51 patients, 24 with paired BM, was able to detect 100% of clinically reported BM biopsy events; therefore, the approach could replace molecular cytogenetics for diagnostic yield and risk classification. Longitudinal sampling of CTCs in 8 patients revealed major clones could be tracked in the blood, with clonal evolution and shifting dynamics of subclones over time. The findings provide proof of concept that CTC detection (e.g., in a liquid biopsy sample) and genomic profiling can be used clinically for monitoring and managing disease in MM.

The Examples provided herein demonstrate that the methods provided in this disclosure allow for the identification and separation of circulating plasma cells of tumor origin from normal cells and facilitate further downstream analysis. The invention is further based, at least in part, upon the discovery that methods of the invention can be used to replace, accompany, or supplant fluorescence in situ hybridization (FISH) and/or cytogenetics analysis (e.g., analyses of bone marrow biopsy samples) in myeloma diagnosis and prognosis. In some embodiments, the methods of the invention are used to monitor disease state.

In various embodiments, the methods of the invention can help physicians in patient management decisions. For example, the methods can be used for prognostication, to inform treatment, and/or to monitor disease status/progression for multiple myeloma patients, asymptomatic monoclonal gammopathy of undetermined significance patients, or smoldering multiple myeloma patients. The methods can be used to inform implementation of precision medicine strategies for overt multiple myeloma patients. In various embodiments, the methods of the invention involve early detection of a cancer or tumor, and/or real-time monitoring and/or assessment of response to a treatment for a cancer or tumor.

Multiple myeloma (MM) has a well described continuum of disease, however, the initial diagnosis of monoclonal gammopathy of undetermined significance (MGUS) or smoldering multiple myeloma (SMM) remains an incidental process through the identification of increased clonal immunoglobulin in the blood. Bone marrow (BM) is a gold standard for diagnosis and monitoring of disease progression, however, BM biopsy is an intrusive and painful procedure with possible secondary complications for patients. Moreover, research-level next generation sequencing (NGS) studies have established complex clonal architecture with the existence of genetic heterogeneity and spatial heterogeneity in multiple myeloma (MM). Thus, localized BM biopsy alone is not always able to represent the full pathology of disease. This presents an urgent need for improvement in robust early detection methods that are biology based, able to capture the full picture of temporal and spatial nature of disease, and ideally being minimally invasive for patients.

Currently diagnosis is only as good as the assays and techniques available, thus improved sensitivity and method development for reliable detection are needed. In the transformation of a normal plasma cell (PC) to proliferating MM cell, known key initiating events including hyperdiploidy and/or translocations are present in virtually all patients. Therefore, copy number changes and structural variants make up a key component of stratification, where these biological events are known to be associated with disease risk and prognosis. FISH has an important role in detecting structural abnormalities, however, was developed long ago and faces limitations such as varied probe sets across clinics or no result when insufficient cells are available. The methods of the invention provide minimally invasive blood biopsies to measure tumor biology-based analytes such as circulating tumor cells (e.g., multiple myeloma cells (CMMCs)) as markers of a disease (e.g., multiple myeloma). Since CMMCs are rare tumor cells that extravasate from the primary BM tumor site to the blood, they present an elegant solution to capture representation of the current major clone of disease.

Additionally, while not intending to be bound by theory, circulating tumor cells (e.g., CMMCs) are intact cells from the primary tumor and therefore amenable to molecular characterization to uncover underlying initiating genetic abnormalities (e.g., clonal events) and prognostic risk factors for actionable outcomes for patients. As blood draws are readily possible and repeatable, there are many advantages for potential clinical use in settings of early detection, real time monitoring and assessing treatment response/clonal evolution.

### Hematological Malignancies

Multiple myeloma (MM) is a plasma cell dyscrasia. Plasma cell dyscrasias are cancers of the plasma cells. They are produced as a result of malignant proliferation of a monoclonal population of plasma cells that may or may not secrete detectable levels of a monoclonal immunoglobulin or paraprotein commonly referred to as M protein. Further non-limiting examples of plasma cell dyscrasias include monoclonal gammopathy of undermined significance (MGUS), smoldering multiple myeloma (SMM), symptomatic multiple myeloma, Waldenstrom macroglobulinemia (WM), amyloidosis (AL), plasmacytoma syndrome (e.g., solitary plasmacytoma of bone, extramedullary plasmacytoma), light chain deposition disease, and heavy-chain disease. MGUS, smoldering MM, and symptomatic MM represent a spectrum of the same disease.

Monoclonal Gammopathy of Undermined Significance (MGUS) MGUS is characterized by a serum monoclonal protein (<30 g/L), <10% plasma cells in the bone marrow, and absence of end-organ damage. Asymptomatic MGUS stage consistently precedes multiple myeloma (MM). MGUS is present in 3% of persons >50 years and in 5% >70 years of age. The risk of progression to MM or a related disorder is 1% per year. Patients with risk factors consisting of an abnormal serum free light chain ratio, non-immunoglobulin G (IgG) MGUS, and an elevated serum M protein (≥15 g/1) have a risk of progression at 20 years of 58%, compared with 37% among patients with two risk factors, 21% for those with one risk factor, and 5% for individuals with no risk factors. The cumulative probability of progression to active MM or amyloidosis is 51% at 5 years, 66% at 10 years and 73% at 15 years; the median time to progression was 4.8 years

Smoldering Multiple Myeloma (SMM) also known as asymptomatic MM is characterized by having a serum immunoglobulin (Ig) G or IgA monoclonal protein of 30 g/L or higher and/or 10% or more plasma cells in the bone marrow but no evidence of end-organ damage. Not intending to be bound by theory, there are 2 different types of SMM: evolving smoldering MM and non-evolving Smoldering MM. Evolving SMM is characterized by a progressive increase in M protein and a shorter median time to progression (TTP) to active multiple myeloma of 1.3 years. Non-evolving SMM has a more stable M protein that can then change abruptly at the time of progression to active multiple myeloma, with a median TTP of 3.9 years.

Symptomatic or Active Multiple myeloma (MM) is a form of cancer that affects a type of white blood cell called the plasma cell. Multiple myeloma appears in the bone marrow, which is the soft tissue inside the bones that makes stem cells. In multiple myeloma, plasma cells, which mature from stem cells and typically produce antibodies to fight germs and other harmful substances, become abnormal. These abnormal cells are called myeloma cells. In 2021, an estimated 34,920 cases of multiple myeloma were diagnosed in the United States and over 12,410 patient deaths associated with multiple myeloma were reported. As the most common type of plasma cell cancer, effective treatment requires an accurate diagnosis and precise treatment.

In embodiments, symptomatic or active MM is characterized by any level of monoclonal protein and the presence of end-organ damage that consists of the CRAB criteria (hypercalcemia, renal insufficiency, anemia, or bone lesions). In some instances, multiple myeloma diagnosis is made using the detection of a biomarker for a myeloma defining event, as described, for example, in Rajkumar, S., et al., The Lancet Oncology, 15:E538-548 (2014), doi: 10.1016/S1470-2045(14)70442-5, the disclosure of which is incorporated herein by reference in its entirety for all purposes. MM is a plasma cell malignancy that characteristically involves extensive infiltration of bone marrow (BM), with the formation of plasmacytomas, as clusters of malignant plasma cells inside or outside of the BM milieu. Consequences of this disease are numerous and involve multiple organ systems. Disruption of BM and normal plasma cell function leads to anemia, leukopenia, hypogammaglobulinemia, and thrombocytopenia, which variously result in fatigue, increased susceptibility to infection, and, less commonly, increased tendency to bleed. Disease involvement in bone creates osteolytic lesions, produces bone pain, and may be associated with hypercalcemia.

### Conventional Detection Methods

To date, the gold standard for characterizing MM disease state has involved a bone marrow biopsy. The present disclosure provides a non-invasive method for characterizing the disease state of a patient. The methods of the invention are suitable for use alone, or if desired, may be used in concert with one or more of the following conventional diagnostic methods.

Traditionally, the initial evaluation of a suspected hematological malignancy (e.g., a monoclonal gammopathy) includes both serum and urine protein electrophoresis with immunofixation to identify and quantify the M protein. The majority of patients are expected to have a detectable M protein, but approximately 1-3% can present with a non-secretory myeloma that does not produce light or heavy chains. True non-secretory myeloma is thus rare, not least because, with the availability of serum free light chain testing, it is recognized that M protein is present. The most common M protein is IgG, followed by IgA, and light-chain-only disease. IgD and IgE are relatively uncommon and can be more difficult to diagnose because their M spikes are often very small. Up to 20% of patients will produce only light chains, which may not be detectable in the serum because they pass through the glomeruli and are excreted in the urine. The present invention provides methods that can also be used to detect and/or characterize a monoclonal gammopathy in a patient.

A standard evaluation of a documented monoclonal gammopathy includes a complete blood count with differential, calcium, serum urea nitrogen, and creatinine. Serum free light chain testing is also a useful diagnostic test (Piehler A. P. et al, Clin. Chem., 54: 1823-30 (2008)). Bone disease is best assessed by skeletal survey. Bone scans are not a sensitive measure of myelomatous bone lesions because the radioisotope is poorly taken up by lytic lesions in MM, as a result of osteoblast inhibition. Magnetic resonance imaging (MRI) is useful for the evaluation of solitary plasmacytoma of bone and for the evaluation of paraspinal and epidural components. 18F-FDG Positron Emission Tomography (PET)/CT scans are more sensitive in the detection of active lesions in the whole body (Fonti R. et al., J. Nucl. Med., 49: 195-200 (2008)). A bone marrow aspiration and biopsy are helpful to quantify the plasma cell infiltrate and adds important prognostic information with cytogenetic evaluation, including fluorescent in situ hybridization (FISH). Additional prognostic information can be obtained with serum B2-microglobulin (B2M) and C-reactive protein (CRP).

The criteria for the diagnosis of MM, SMM, and MGUS are detailed in Table 1 below. Distinction among these disease states informs treatment decisions and prognostic recommendations.

TABLE 1
Conventional criteria for the diagnosis of MM, SMM, and MGUS

| Disorder | Disease definition |
|---|---|
| MGUS | Serum monoclonal protein level <3 g/dL, |
| | bone marrow plasma cells 10%, and absence |
| | of end-organ damage, such as lytic bone |
| | lesions, anemia, hypercalcemia, or renal |
| | failure, that can be attributed to a plasma cell |
| | proliferative disorder. |
| SMM | Serum monoclonal protein (IgG or IgA) level ≥3 |
| | g/dL and/or bone marrow plasma cells .10%, |
| | absence of end-organ damage, such as |
| | lytic bone lesions, anemia, hypercalcemia, or |
| | renal failure that can be attributed to a plasma |
| | cell proliferative disorder. |
| | Alternatively or additionally, SMM may be |
| | defined by the presence or absence of |
| | biomarkers (e.g., Myeloma Defining Event |
| | biomarkers), by bone-marrow plasma cell |
| | infiltration ≥60%, by serum-free light chain |
| | ratio ≥100, and/or by >1 focal lesion in the |
| | skeleton on magnetic resonance imaging |
| | analysis (see, Rajkumar SV, et al. |
| | International Myeloma Working Group |
| | updated criteria for the diagnosis of multiple |
| | myeloma. Lancet Oncol 15, e538-548 |
| | (2014)). In some instances, SMM may be |
| | associated with organ damage. |
| MM | Bone marrow plasma cells ≥10%, presence of |
| | serum and/or urinary monoclonal protein |
| | (except in patients with true nonsecretory |
| | multiple myeloma), plus evidence of lytic |
| | bone lesions, anemia, hypercalcemia, or renal |
| | failure that can be attributed to the underlying |
| | plasma cell proliferative disorder. |

Conventional staging systems involve the following. The most widely used myeloma staging system since 1975 has been the Durie-Salmon, in which the clinical stage of disease is based on several measurements including levels of M protein, serum hemoglobin value, serum calcium level, and the number of bone lesions. The International Staging System (ISS), developed by the International Myeloma Working Group is now also widely used (Greipp P R. Et al, J. Clin. Oncol, 23:3412-20 (2005)). ISS is based on two prognostic factors: serum levels of B2M and albumin, and is comprised of three stages: B2M 3.5 mg/L and albumin 3.5 g/dL (median survival, 62 months; stage I); B2M <3.5 mg/L and albumin <3.5 g/dL or B2M 3.5 to <5.5 mg/L (median survival, 44 months; stage II); and B2M 5.5 mg/L (median survival, 29 months; stage III). With an increased understanding of the biology of myeloma, other factors have been shown to correlate well with clinical outcome and are now commonly used. For example, cytogenetic abnormalities as detected by FISH techniques have been shown to identify patient populations with very different outcomes. For instance, loss of the long arm of chromosome 13 is found in up to 50% of patients and, when detected by metaphase chromosome analysis, is associated with poor prognosis. In addition, a hypodiploid karyotyped t(4;14), and −17pl3.1 is typically associated with poor outcome, while the t(11;14) and hypodiploidy are associated with improved survival (Kyrtsonis M. C. et al., Semin. Hematol, 46: 110-7, (2009)).

Purification and/or Counting of Circulating Tumor Cells

Disease state can also be assessed by characterizing the genomes of MM cells isolated from the peripheral blood of a subject. Such characterization is facilitated by the isolation of circulating tumor cells (e.g., circulating multiple myeloma cells (CMMCs)) from a sample (e.g., a liquid biopsy, such as a peripheral blood sample). In embodiments, genomic DNA from the circulating tumor cells is isolated and sequenced. In various embodiments, the MM cells are purified using an immunophenotype-based enrichment technique, such as Fluorescence-activated cell sorting (FACS) or CellSearch™.

In embodiments, the methods of the disclosure involve enumeration of circulating tumor cells. Such enumeration can be used for characterizing disease state (e.g., multiple myeloma (MM), smoldering multiple myeloma (SMM), monoclonal gammopathy of undermined significance (MGUS)).

In various embodiments, the methods of the invention involve sorting and/or counting cells (e.g., circulating tumor cells, such as circulating multiple myeloma cells (CMMCs)) obtained from a liquid biopsy (e.g., a blood sample) from a subject. The cells can be sorted and counted using any suitable method known in the art, such as an immunophenotype-based enrichment method. The cells can be sorted using a commercially available kit, such as the Silicon Biosystems Circulating Multiple Myeloma Cell Assay kit, which can be used in combination with a CellSearch™ system. Non-limiting examples of immunophenotype-based enrichment methods include a CellSearch™ system (an immunomagnetic and immunofluorescence imaging technology), and fluorescence activated cell sorting (FACS; e.g., high-sensitivity fluorescence-activated cell sorting). The immunophenotypes CD138+ and/or CD38+ can be used to select for plasma cells and the immunophenotypes CD45− and/or CD19− can be used to exclude non-PC leukocytes from a selection. In embodiments, a DAPI stain can be used to select for and/or detect nucleated cells.

In embodiments, the methods of the invention involve sequencing of DNA isolated from the enriched cells according to methods described herein. The sequence data obtained according to the methods of the invention allows for mutational analyses of iterating, clinically relevant and prognostic events of multiple myeloma including, as non-limiting examples, structural variation, copy number variation, and single nucleotide variation. In embodiments, the cells are enriched or isolated from a large background of mononuclear cells.

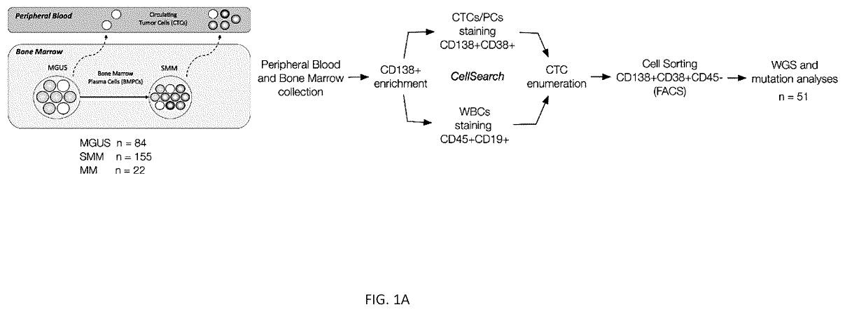

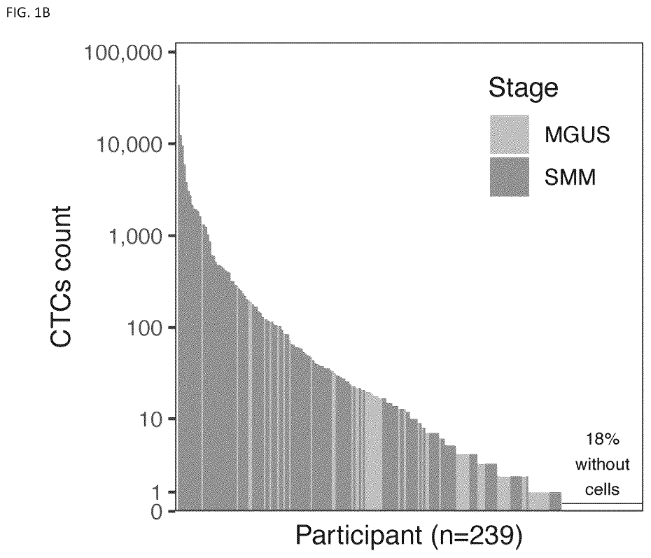

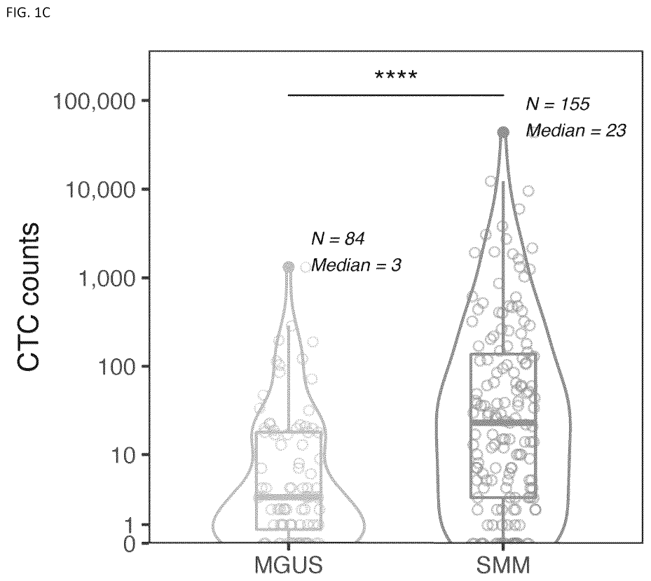

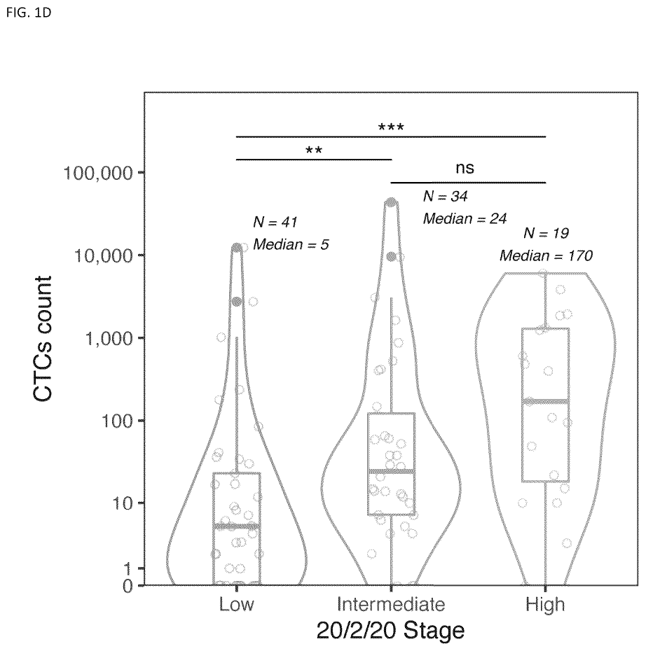

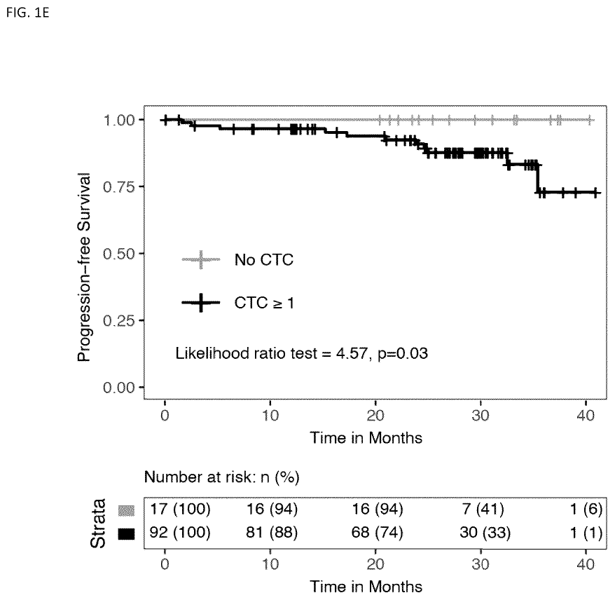

In various aspects, the invention provides methods (see, e.g., FIG. 1) that involve isolation or enrichment of a small number of purified circulating tumor cells (e.g, about or at least about 2 cells, 3 cells, 4 cells, 5 cells, 6 cells, 7 cells, 8 cells, 9 cells, 10 cells, 20 cells, 30 cells, 40 cells, 50 cells, 60 cells, 70 cells, 80 cells, 90 cells, 100 cells, 200 cells, 300 cells, 400 cells, 500 cells, 600 cells, 700 cells, 800 cells, 900 cells, 1000 cells, 2000 cells, 3000 cells, 4000 cells, 5000 cells, 6000 cells, 7000 cells, 8000 cells, 9000 cells, 1000 cells, 2000 cells, 3000 cells, 4000 cells, 5000 cells, 6000 cells, 7000 cells, 8000 cells, 9000 cells, 1000 cells, 10000 cells, 20000 cells, 30000 cells, 40000 cells, 50000 cells, 60000 cells, 70000 cells, 80000 cells, 90000 or 100000 cells). In some instances, the methods further involve purification of genomic DNA from the cells, and sequencing of the genomic DNA without any whole-genome amplification step.

In embodiments, a collection of sorted cells (e.g., a “minipool”) contains about or at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 1000, 2000, 3000, 4000, 5000, 6000, 7000, 8000, 9000, 1000, 10000, 20000, 30000, 40000, 50000, 60000, 70000, 80000, 90000 or 100000 cells (e.g., circulating tumor cells, CMMCs, and/or plasma cells). In some instances, the collection of sorted cells contains no more than about 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 1000, 2000, 3000, 4000, 5000, 6000, 7000, 8000, 9000, 1000, 10000, 20000, 30000, 40000, 50000, 60000, 70000, 80000, 90000 or 100000 cells (e.g., circulating tumor cells, CMMCs, and/or plasma cells). In embodiments, the collection of sorted cells contains a fraction of tumor cells equal to about or at least about 5%, 10%, 15%, 20%, 25%, 30%, 35%, 40%, 45%, 50%, 55%, 60%, 65%, 70%, 75%, 80%, 85%, 90%, 95%, 99%, or 100%. In some instances, the collections of sorted cells include only plasma cells (e.g., circulating tumor cells or CMMCs). In embodiments, the collection of sorted cells contains no more than about 10, 100, 1000, or 10000 leukocytes and/or other non-plasma cells. In various embodiments, the cells are intact cells.

### Sequencing and Analysis

In embodiments the methods of the invention involve sequencing genomic DNA obtained from a collection of sorted cells prepared according to the methods provided herein. In embodiments, the method for sequencing the genomic DNA does not involve any whole-genome amplification step.

In embodiments of the methods provided herein, next-generation sequencing (NGS) of genomic DNA from cells isolated or enriched from a liquid biopsy sample allows for capture of the genetic abnormalities of MM, similar to that detected in standard BM biopsy sample analysis alone. The methods of the invention enable quantitative disease monitoring for patients in the clinic at regular intervals. In various instances, the methods of the invention involve enriching and selecting circulating tumor cells (e.g., CMMCs) from a large background of mononuclear cells, and subsequently extracting nucleic acids from this minute cell fraction to allow for subsequent molecular characterization of the circulating tumor cells.

Any suitable method for isolation of DNA from the cells may be used in the methods of the invention (e.g., proteinase K-based purification methods). Various kits are commercially available for the purification of polynucleotides from a sample and are suitable for use in the methods of the invention (e.g., an Arcturus PicoPure DNA Extraction Kit, Thermo Fisher Scientific). In an embodiment, the genomic DNA is purified using a proteinase K digestion-based technique (e.g., Arcturus PicoPure DNA Extraction Kit, Thermo Fisher Scientific)

In embodiments, the extracted DNA is used to prepare a sequencing library. Methods for preparing libraries of polynucleotides for sequencing are known to one of skill in the art. Library preparation can include the addition of nucleotide bar codes to the library polynucleotides according to methods known in the art. Libraries can be prepared using commercially available kits.

Not intending to be bound by theory, methods involving whole-genome amplification of DNA from recovered cells, followed by sequencing, only allows for reliable copy number variant analysis as a consequence of stochastic variation during the amplification process. Whole-genome amplification (WGA) may introduce one or more of amplification bias, artifacts, allelic distortions, and non-uniformity in genome coverage. Thus, methods involving a whole-genome amplification step may provide certain challenges in performing unbiased whole-genome analysis on small inputs of DNA (e.g., picogram-levels of DNA and/or genomic DNA isolated from 1 to 100 cells), optionally where the DNA is derived from a liquid biopsy sample.

In embodiments, sequencing of the genomic DNA involves construction of a sequencing library without any whole-genome DNA amplification. In some instances, the sequencing library is prepared using enzymatic fragmentation-based techniques (e.g., NEBNext Ultra II FS, New England Biolabs). In various instances, the sequencing libraries are prepared using picogram levels of genomic DNA; for example, about or less than about 1 pg, 2 pg, 3 pg, 4 pg, 5 pg, 6 pg, 7 pg, 8 pg, 9 pg, 10 pg, 20 pg, 30 pg, 40 pg, 50 pg, 60 pg, 70 pg, 80 pg, 90 pg, 100 pg, 200 pg, 300 pg, 400 pg, 500 pg, 600 pg, 700 pg, or 900 pg. In various embodiments, the methods provided herein involve no whole-genome amplification step. Not intending to be bound by theory, a single cell typically contains around 6 pg of double-stranded DNA.

The extracted DNA may be sequenced using any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide and/or sequencing library can be carried out using any suitable commercially available sequencing technology. In another embodiment, the sequencing of a polynucleotide is carried out using chain termination method of DNA sequencing (e.g., Sanger sequencing). In yet another embodiment, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing. In embodiments, the polynucleotide is sequenced using HiSeq2500 or Novaseq6000.

In embodiments, the sequencing is to a coverage of about or at least about 0.001, 0.01, 0.05, 0.1, 0.2, 0.3, 0.4, 0.5, 0.75, 1, 2, 3, 4, 5, 6, 7, 8, 9, 10, 20, 30, 40, 50, 60, 70, 80, 90, or 100×, where a sequencing coverage of 0.01 indicates that a DNA sample has been sequenced such that the amount of DNA sequenced is equivalent in size to about 1% of the corresponding genome from which the DNA sample is derived. In embodiments, the sequencing is to a coverage of no more than about 0.001, 0.01, 0.05, 0.1, 0.2, 0.3, 0.4, 0.5, 0.75, 1, 2, 3, 4, 5, 6, 7, 8, 9, 10, 20, 30, 40, 50, 60, 70, 80, 90, or 100×.

In embodiments, the methods of the disclosure further involve analyzing sequence data obtained through the sequencing of a polynucleotide and/or sequencing library. The analysis can involve the detection of clinically relevant and/or prognostic events, such as driver mutations, single nucleotide variation, and/or chromosomal rearrangements (e.g., structural variation or copy number variation) associated with a multiple myeloma. Non-limiting examples of clinically relevant and/or prognostic events that may be detected include aneuploidy (e.g., hyperploidies, such as trisomies, or tetrasomies; and monoploidies), translocations (e.g., t(4;14), t(6;14), t(8;14), t(11;14), t(14;16), t(14;20), t(2;8), and t(8;22)), chromosomal arm gains or deletions (e.g., chromosome 1q gain, chromosome 1p deletion, chromosome 13q deletion, chromosome 16q deletion, and chromosome 17p deletion), and/or driver mutations (e.g., non-silent mutations to KRAS or NRAS, and/or mutations to DIS3, FAM46C, BRAF, and/or TP53). The event detected can be predictive of risk and MM progression. In embodiments, a driver mutation is selected from a G12, G13, Q61, K117, or A146 alteration to KRAS or NRAS. In some instances, translocations are detected using density-based graph clustering of sequencing reads supporting oncogenic structural rearrangements. In embodiments, structural rearrangements are detected using sequencing read pairs or single-sequencing reads from both chromosomes of a translocation (e.g., from both chromosome 11 and chromosome 14). In embodiments, a driver mutation is selected from any non-silent mutation (e.g., a G12, G13, Q61, K117, or A146 alteration) to KRAS or NRAS. In some instances, a driver mutation is selected from any non-silent mutation to any one or more of the following genes: KRAS, NRAS, DIS3, BRAF, FAM46C, TP53, MYC, MAX, IGLL5, TRAF3, DUSP2, TCL1A, TRAF2, CYLD, LTB, HIST1H1E, BCL7A, SP140, NFKBIA, EGR1, PABPC1, PRKD2, TBC1D29, IRF4, RB1, TGDS, PTPN11, FUBP1, RPL5, FGFR3, SAMHD1, ACTG1, HIST1H1B, NFKB2, KMT2B, KLHL6, RASA2, PIM1, PRDM1, DTX, SETD2, BHLHE41, RPL10, BTG1, RPS3A, CCND1, RPRD1B, HIST1H1D, ZNF292, RFTN1, CDKN1B, LCE1D, XBP1, IRF1, POT1, HIST1H2BK, ABCF1, ZFP36L1, TET2, ARID2, KDM6A, EP300, ARID1A, NCOR1, HUWE1, CDKN2C, SF3B1, ATM, NF1, CREBBP, DNMT3A, MAFB, MAF, KDM5C, UBR5, PIK3CA, IDH1, MCL1, BIRC2, MLL1, MLL2, MAML2, MAN2C1, IDH2, KMT2C, and ATRX.

The sequence data obtained according to the methods of the invention allows for the detection and quantification of genetic abnormalities in genomic DNA of circulating tumor cells (e.g., circulating multiple myeloma cells (CMMCs)) from patients with hematological malignancies (e.g., monoclonal gammopathies).

### Treatments

Methods of inhibiting and/or treating cancer and tumors (e.g., a multiple myeloma) in a subject with cancer or a predisposition for developing cancer as identified by methods of the disclosure are also contemplated. Methods described herein are useful as clinical or companion diagnostics for therapies or can be used to guide treatment decisions based on clinical response/resistance. For example, a subject having a translocation t(4;14), t(14;16), t(14;20), or del(17p) can advantageously be treated using a tandem autologous stem cell transplant (ASCT) in multiple myeloma patients, optionally instead of single ASCT (see, e.g., Kumar 2017 Nat Rev Dis Prim doi:10.1038/nrdp.2017.46). In an embodiment, a subject having a translocation t(11;14) can advantageously be treated using venetoclax. In some instances, a subject with 17p or that is classified as being at high risk is treated with a tandem transplant, immunotherapy, or consolidation therapy (e.g., radiation therapy, stem cell transplant, or treatment with a chemotherapeutic agent). In some embodiments, a subject classified as being at low or intermediate risk may stop therapy without prolonged maintenance if they are in minimal residual disease (MRD).

Frontline therapy for MM includes either conventional chemotherapy or high-dose chemotherapy (HDT) supported by autologous or allogeneic stem cell transplantation (SCT), depending on patient characteristics such as performance status, age, availability of a sibling donor, comorbidities, and, in some cases, patient and physician preferences. Other treatments include: bortezomib, thalidomide, lenalidomide, dexamethasone, cyclophosphamide, melphalan, and stem cell transplant. For a patient under 70 years of age, autologous stem cell transplant is proposed after induction.

Non-limiting examples of agents suitable for use to treat a multiple myeloma include a chemotherapeutic agent, radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, tamoxifen, taxol, topotecan hydrochloride, trastuzumab, vinblastine, vincristine and vinorelbine tartrate. Further non-limiting examples of chemotherapeutic agents include an alkylating agent (e.g. busulfan, chlorambucil, cisplatin, cyclophosphamide (Cytoxan), dacarbazine, ifosfamide, mechlorethamine (mustargen), and melphalan), a topoisomerase inhibitor, an antimetabolite (e.g. 5-fluorouracil (5-FU), cytarabine (Ara-C), fludarabine, gemcitabine, and methotrexate), an anthracycline, an antitumor antibiotic (e.g. bleomycin, dactinomycin, daunorubicin, doxorubicin (Adriamycin), and idarubicin), an epipodophyllotoxin, nitrosureas (e.g. carmustine and lomustine), topotecan, irinotecan, doxorubicin, etoposide, mitoxantrone, bleomycin, busulfan, mitomycin C, cisplatin, carboplatin, oxaliplatin and docetaxel.

In embodiments, response to therapy is measured using the methods provided herein (e.g., through molecular characterization of circulating multiple myeloma cells). In embodiments, response to therapy is measured by a reduction in M protein levels in serum and/or urine and the reduction in size or disappearance of plasmacytomas. The international uniform response criteria for by have expanded upon the European Group for Blood and Marrow Transplantation criteria to provide a more comprehensive evaluation system (Dure B. G. et al., Leukemia, 20: 1467-73 (2006)). Importantly, achievement of response has been associated with improved survival in SCT trials with high-dose therapy. Similarly, time to progression (TTP) has been shown to be an important surrogate for improved survival. Despite high response rates to frontline therapy, virtually all patients eventually relapse. Table 2 shows the international uniform response criteria for MM.

TABLE 2
International uniform response criteria for multiple myeloma (MM)

| Response | |
|---|---|
| Subcategory | Response Criteria |
| CR (complete | Negative immunofixation on the serum and urine and disappearance of |
| response) | any soft tissue plasmacytomas and ≤5% plasma cells in bone marrow |
| sCR (stringent | CR as described above, plus: |
| complete response) | normal free light chain (FLC) ratio and absence of clonal cells in bone |
| | marrow by immunohistochemistry or immunofluorescence |
| VGPR (very good | Serum and urine M-protein detectable by immunofluorescence but not |
| partial response) | on electrophoresis or 90% or greater reduction in serum M-protein plus |
| | urine M-protein level <100 mg per 24 hours |
| PR (partial | ≥50% reduction of serum M-protein and reduction in 24-h urinary M- |
| response) | protein by ≥90% or to <200 mg per 24 h |
| | If the serum and urine M-protein are unmeasurable, a ≥50% decrease |
| | in the difference between involved and uninvolved FLC levels is |
| | required in place of the M-protein criteria |
| | If serum and urine M-protein are unmeasurable, and serum free light |
| | assay is also unmeasurable, ≥50% reduction in plasma cells is required |
| | in place of M-protein, provided baseline bone marrow plasma cell |
| | percentage was ≥30% |
| | In addition to the above listed criteria, if present at baseline, a ≥50% |
| | reduction in the size of soft tissue plasmacytomas is also required. |
| SD (stable disease) | Not meeting criteria for CR, VGPR, PR or progressive disease |

In embodiments, the subject has been diagnosed with cancer or is at risk of developing a multiple myeloma.

For therapeutic use, administration of an agent can begin at the detection or surgical removal of tumors. This can be followed by boosting doses until at least symptoms are substantially abated and for a period thereafter.

The pharmaceutical compositions for therapeutic treatment are intended for parenteral, topical, nasal, oral or local administration. Preferably, the pharmaceutical compositions are administered parenterally, e.g., intravenously, subcutaneously, intradermally, or intramuscularly. The disclosure provides compositions for parenteral administration which comprise a solution of a suitable agent dissolved or suspended in an acceptable carrier, preferably an aqueous carrier. A variety of aqueous carriers may be used, e.g., water, buffered water, saline, glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, triethanolamine oleate, etc.

In an advantageous embodiment, the cancer therapeutic is an immunotherapeutic (e.g., an antibody). The cancer therapeutic can be a chimeric antigen receptor (CAR) T cell. The immunotherapeutic may be a cytokine therapeutic (such as an interferon or an interleukin), a dendritic cell therapeutic or an antibody therapeutic, such as a monoclonal antibody. In a particularly advantageous embodiment, the immunotherapeutic is a neoantigen (see, e.g., U.S. Pat. No. 9,115,402 and US Patent Publication Nos. 20110293637, 20160008447, 20160101170, 20160331822 and 20160339090).

### Monitoring Hematological Malignancy Stage

Subjects being treated for a hematological malignancy (e.g., a monoclonal gammopathy) may be characterized using any of the methods described herein. Cells characteristic of a hematological malignancy typically display alterations in their genome compared to corresponding normal reference cells. Genetic alterations (e.g., mutations, chromosomal rearrangements, or aneuploidy) are correlated with multiple myeloma and related pathologies (e.g., MGUS, SMM).

In embodiments, the methods of the invention are used to monitor a patient. In some instances, monitoring of a patient involves characterizing circulating tumor cells (e.g., CMMCs) from a subject according to the methods provided herein every or at least about every 1 day, 1 week, 2 weeks, 3 weeks, 4 weeks, 5 weeks, 6 weeks, 7 weeks, 8 weeks, 9 weeks, 10 weeks, 11 weeks 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, or year, optionally over a period of at least about 1 week, 2 weeks, 3 weeks, 4 weeks, 5 weeks, 6 weeks, 7 weeks, 8 weeks, 9 weeks, 10 weeks, 11 weeks 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, or a year.

In these methods, a biological sample (e.g., a liquid biopsy) is obtained from the subject and circulating tumor cells isolated from the biological sample are characterized according to the methods provided herein. The biological sample can be, e.g., a body fluid such as blood or plasma, or a sample from a tumor from the subject. Typically, the biological sample is a blood sample (e.g., a peripheral blood (PB) sample). In various embodiments, a method for identifying the stage of a multiple myeloma involves counting the number of circulating multiple myeloma cells (CMMCs) in the biological sample. An elevated number of circulating tumor cells (e.g., CMMCs) relative to a reference (e.g., a healthy subject) is indicative of a later stage of multiple myeloma. In embodiments, a patient with SMM has a higher level of circulating tumor cells (CTCs) in a peripheral blood sample than a patient with MGUS. Also, in some embodiments, elevated CTCs in a peripheral blood sample taken from a patient relative to a reference is indicative of a higher risk stage for the multiple myeloma (e.g., a 2/20/20 risk stage). For example, in embodiments, a subject with SMM has a CTC count of from about 1 to about or at least about 1, 2, 3, 4, 5, 6, 7, 8, 9, 10, 11, 12, 13, 14, 15, 16, 17, 18, 19, or 20 cells and a subject with SMM has a CTC count of about or at least about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000, and a subject with MM has a CTC count of about or at least about 500, 600, 700, 800, 900, 1000, 1100, 1200, 1300, 1400, 1500, or 2000. In embodiments, a low 2/20/20 risk stage is associated with a CTC count of less than about 1, 2, 3, 4, 5, 6, 7, 8, 9, 10, 11, 12, 13, 14, 15, 16, 17, 18, 19, 20, 25, or 30 cells, an intermediate 2/20/20 risk is associated with a CTC count of from about 5, 10, 15, 20, 25, 30, 35, 40, 45, or 50 cells to about 50, 60, 70, 80, 90, 100, 200, 300, 400, 500, 600, 700, 800, 900, 1000, or 10,000 cells, and a high 2/20/20 risk is associated with a CTC count of greater than about 500, 600, 700, 800, 900, 1000, 1100, 1200, 1300, 1400, 1500, 2000, 3000, 4000, or higher. In embodiments, the cells are counted using a 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, or more peripheral blood sample.

Measuring the levels of the CTCs is also useful in determining whether a particular treatment is working for the subject. The levels of CTCs may be measured at any point where a health care practitioner expects the treatment the subject has been receiving to have begun to be effective in controlling the malignancy (e.g., 1 week, 2 weeks, 3 weeks, 1 month, two months, three months, four months, five months, six months, between six months and 1 year, at 1 year). The level of the CTCs that are found to be elevated or decreased in the MGUS, SMM, or symptomatic stage of MM are measured in the subject's biological sample obtained before and after treatment. If these CTC levels are similar to the CTC levels from an advanced stage of the malignancy or if the CTC levels remain the same as the stage of the disease when the subject began treatment, the treatment is determined to have been ineffective and a new treatment is considered.

For example, consider a patient at the MGUS stage of MM who is receiving treatment for the malignancy. A biological sample is obtained before and after treatment from the patient. CTC levels at the different stages of the malignancy are measured in both the pre- and post-treatment samples. If after treatment, the patient shows CTC level that resembles the SMM or active MM stage of the disease, or if the CTC level remains unaltered compared to pre-treatment, the treatment regimen that the patient was on is deemed ineffective and a new treatment is administered to the subject. If after treatment, the patient shows CTC levels that resembles a subject who does not have a multiple myeloma (e.g., MGUS, SMM, or MM) or if the CTC levels are lower than before treatment, the patient is continued on the treatment regimen that the patient was receiving.

### Subject Management

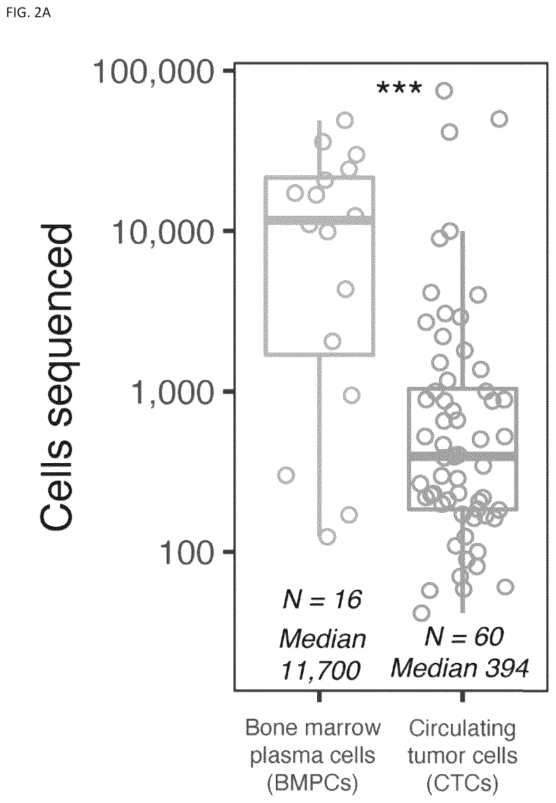

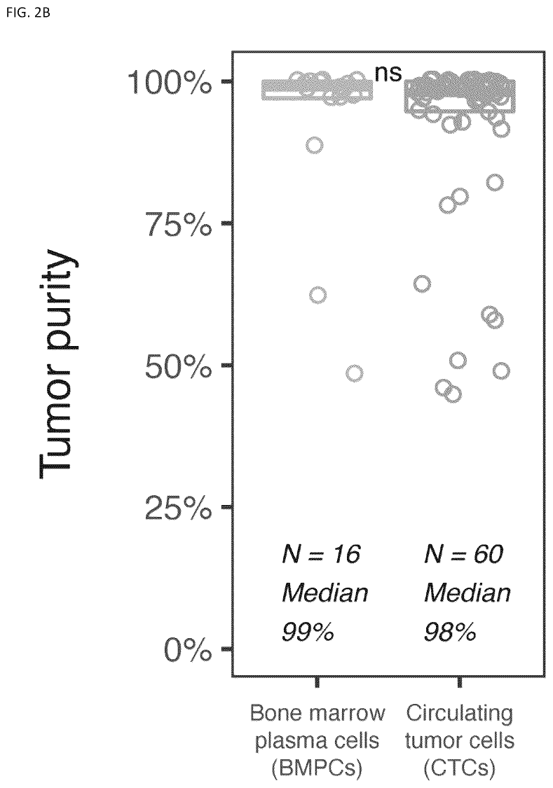

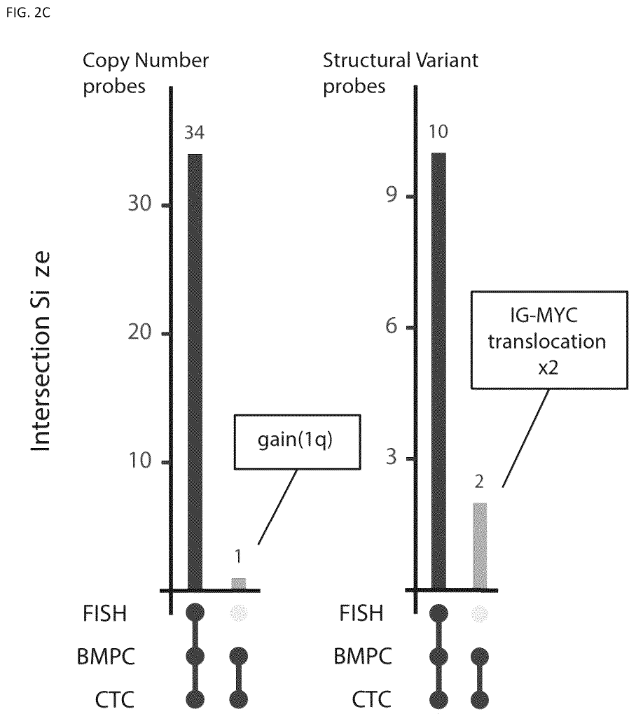

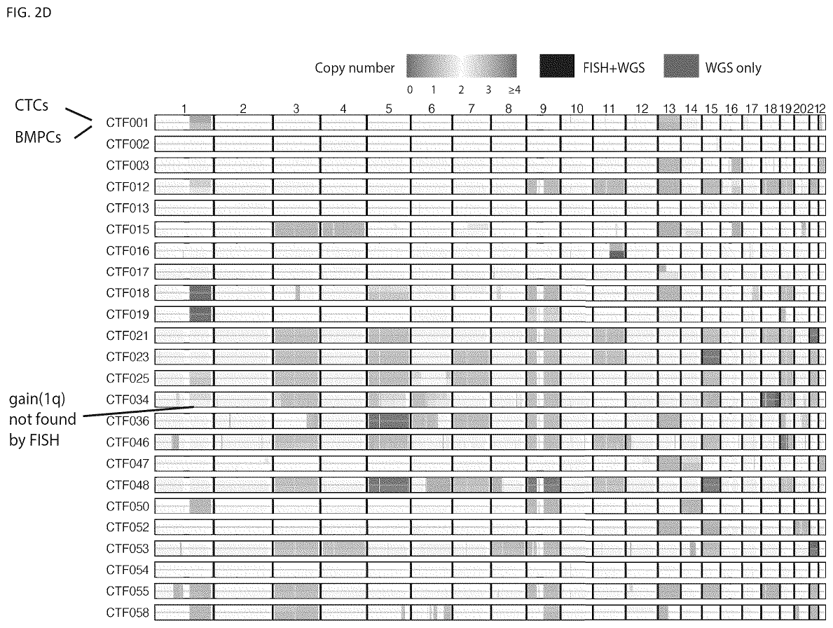

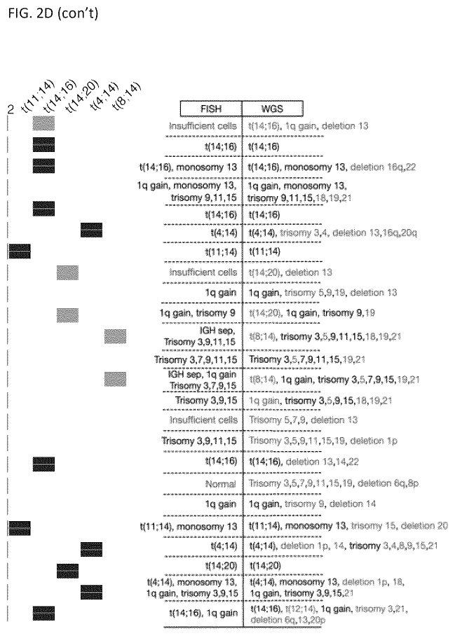

In certain embodiments, the methods of the invention involve managing subject treatment based on disease status (e.g., complete remission, partial remission, resistant disease, stable disease) or based on characterization of CTCs from the subject for an alteration. Such management includes referral, for example, to a qualified specialist (e.g., an oncologist). In one embodiment, if a physician makes a diagnosis of a multiple myeloma (MM), then a certain regime of treatment, such as prescription or administration of therapeutic agent might follow. Alternatively, a diagnosis of non-cancer might be followed with further testing to determine a specific disease that the patient might be suffering from or to determine whether a multiple myeloma in the subject has progressed (e.g., from one state in the development of a multiple myeloma to another, such as from MGUS to SMM or from SMM to MM; see FIG. 2). In some embodiments, subject management involves routine monitoring of multiple myeloma (MM) status in the patient through regular (e.g., weekly, monthly, yearly, etc.) characterization of CTCs from the patient. Also, if the diagnostic test gives an inconclusive result on cancer status, further tests may be called for.

Additional embodiments of the invention relate to the communication of assay results or diagnoses or both to technicians, physicians, or patients. In certain embodiments, computers will be used to communicate assay results or diagnoses or both to interested parties, e.g., physicians and their patients. In some embodiments, the assays will be performed, or the assay results analyzed in a country or jurisdiction which differs from the country or jurisdiction to which the results or diagnoses are communicated.

The disease state or treatment of a patient having a cancer or disease can be monitored using the methods and compositions of this invention. In one embodiment, the response of a patient to a treatment can be monitored using the methods and compositions of this invention. Such monitoring may be useful, for example, in assessing the efficacy of a particular treatment in a patient. Treatments amenable to monitoring using the methods of the invention include, but are not limited to, chemotherapy, radiotherapy, immunotherapy, and surgery.

### Hardware and Software

The present disclosure also relates to a computer system involved in carrying out the methods of the disclosure relating to both computations and sequencing. The methods described herein, analyses can be performed on general-purpose or specially-programmed hardware or software. One can then record the results (e.g., characterization of a CTC) on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In aspects, the analysis is performed by an algorithm. The analysis of sequences will generate results that are subject to data processing. Data processing can be performed by the algorithm. One of ordinary skill can readily select and use the appropriate software and/or hardware to analyze a sequence.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations (e.g., aneuploidy, translocations, and/or MM driver mutations) observed in the data. In aspects, software used to analyze the data can include code that applies an algorithm to the analysis of the results. The software can also use input data (e.g., sequence) to characterize CTCs.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Kits

The disclosure also provides kits for use in characterizing a biological sample from a subject. Kits of the instant disclosure may include one or more containers comprising an agent for enriching/isolating and/or characterization of CTCs (e.g., CMMCs) and/or for treatment of a multiple myeloma (MM). In some embodiments, the kits further include instructions for use in accordance with the methods of this disclosure. In some embodiments, these instructions comprise a description of use of the agent to enrich/isolate and/or characterize CTCs and/or use of the agent for treatment of a multiple myeloma (MM). In some embodiments, the instructions comprise a description of how to isolate polynucleotides from a sample and/or to characterize CTCs. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Detection and Enumeration of CTCs Correlate with Precursor Disease Pathology

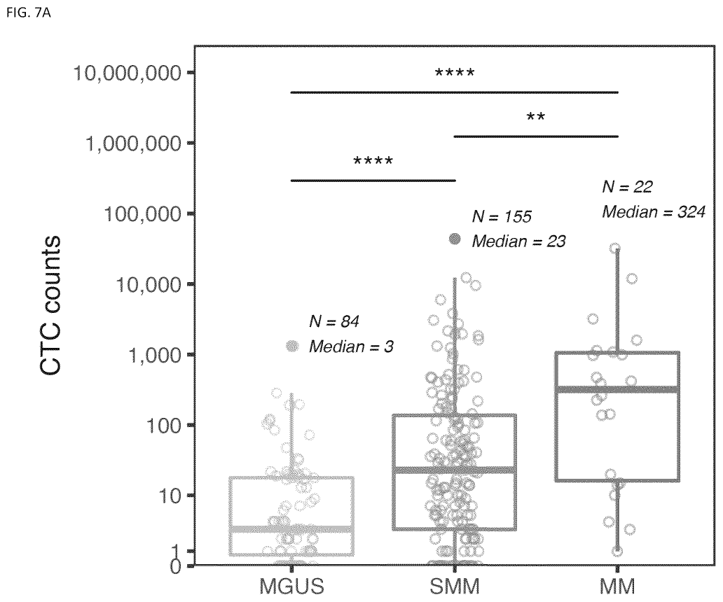

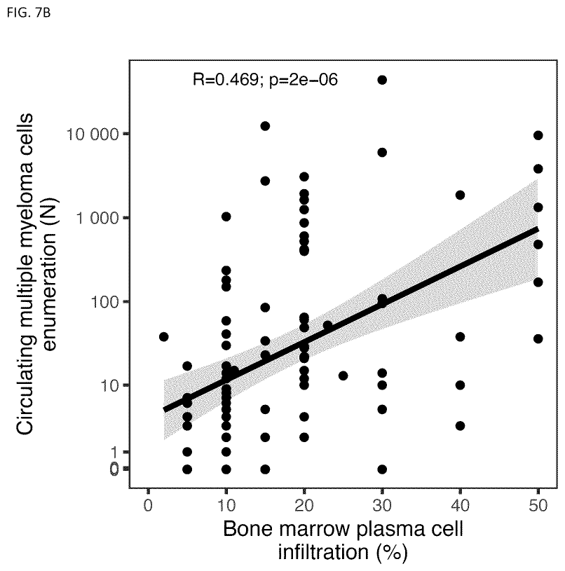

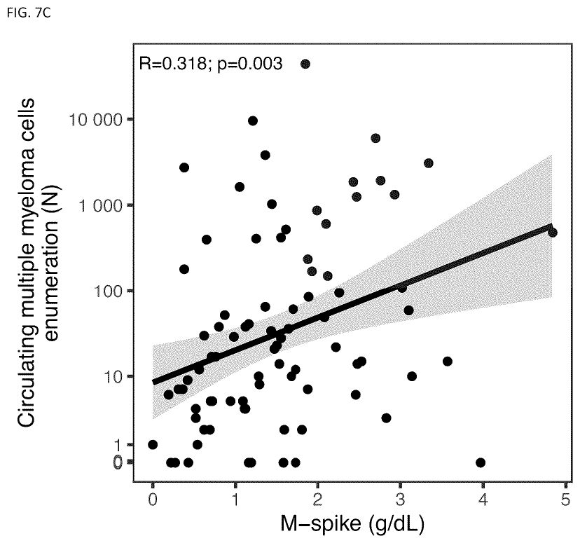

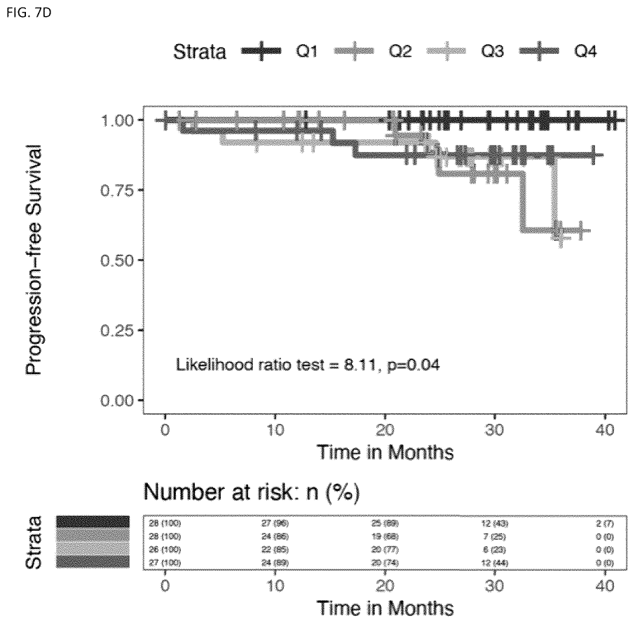

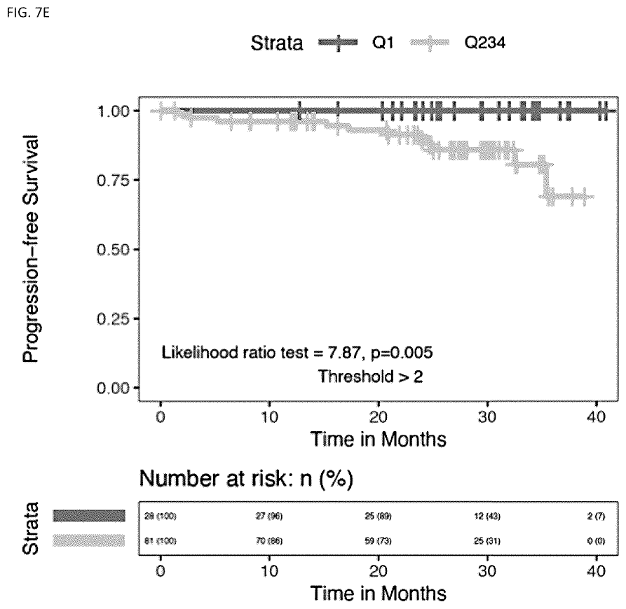

To evaluate the frequency of CTCs in precursor disease stages, peripheral blood was collected from 239 untreated patients across asymptomatic stages of MM, including 84 MGUS and 155 SMM, for the enrichment and capture of CTCs using the CellSearch platform with the Circulating Multiple Myeloma Cell Assay (Menarini Silicon Biosystems), which requires 4 mL of blood per test [FIG. 1A]. The majority of precursor patients showed evidence of CTCs, with one or more CTC detected in 82% of enrolled patients, with 75% of MGUS (63 patients) and 86% of SMM (134 patients) having successful enumeration [FIG. 1B]. An increase in the number of CTCs was observed between MGUS and SMM disease stages, with a median count of 3 (range 0 to 1,328) and 23 (range 0 to 43,836), respectively (P<0.0001) [FIG. 1C]. A cohort of newly diagnosed MM patients was also collected (n=22), with a median enumeration of 324 (range 1 to 32,071) at the overt disease stage [FIG. 7A].

Whether CTC counts correlated with clinical parameters which are monitored for signs of progression, or risk stratification in SMM, CTCs were assessed to determine whether an association with current staging or disease risk is evident. A moderate positive correlation was observed between the number of CTCs enumerated from patients and their BMPC % (r=0.47, 95% CI, 0.29 to 0.61, P<0.0001) [FIG. 7B], and to a lesser extent with the M-spike concentration (r=0.32, 95% CI, 0.11 to 0.50, P=0.003) [FIG. 7C]. A proportion of SMM patients in the cohort had clinical BM biopsy performed (N=94) with results available for the assessment of risk classification using the International Myeloma Working Group 2/20/20 model (20). Enumeration of CTCs correlated with this model, where a higher CTC count was associated with an increased risk group based on the 3-risk factor model, with a median of 5, 24 and 170 CTCs detected in low, intermediate, and high-risk SMM groups, respectively [FIG. 1D]. The median CTC count in low-risk SMM was noted to be similar to that found in MGUS, fittingly reflective of early disease categorization. CTC counts were found to be significantly increased at intermediate (P<0.01) and high-risk stages (P<0.01) compared to the low-risk group. Survival analysis using the Kaplan-Meier method showed SMM patients with detected CTCs have higher chances of progression (CTC count ≥¼ mL of blood, likelihood ratio test statistic 4.57, P=0.03) [FIG. 1E] over a median follow-up time of 27 months (IQR, 21 to 32 months). The association remained true when comparing quartiles of CTC counts (P=0.04) [FIG. 7D], and more specifically the lowest Q1 (0 to 2 CTC/4 mL of blood) against Q2, Q3 and Q4 (P=0.005) [FIG. 7E]. Taken together, these data illustrate that as tumor burden of disease increases, there is an increase in release and trafficking of tumor cells from the BM to the PB which may be predictive of disease staging and monitored by enumeration of CTCs.

### Example 2: MinimuMM-Seq Reveals CTCs Reflect the Major BM Clone and could Replace Molecular Cytogenetics

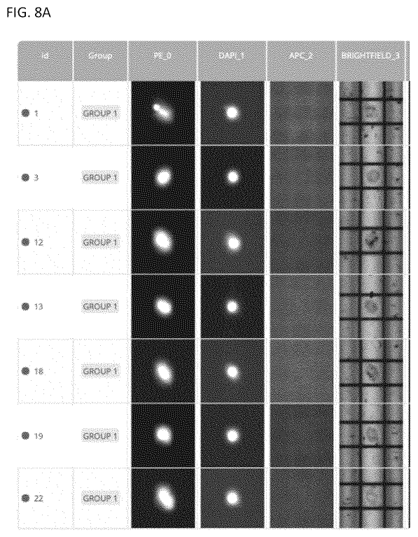

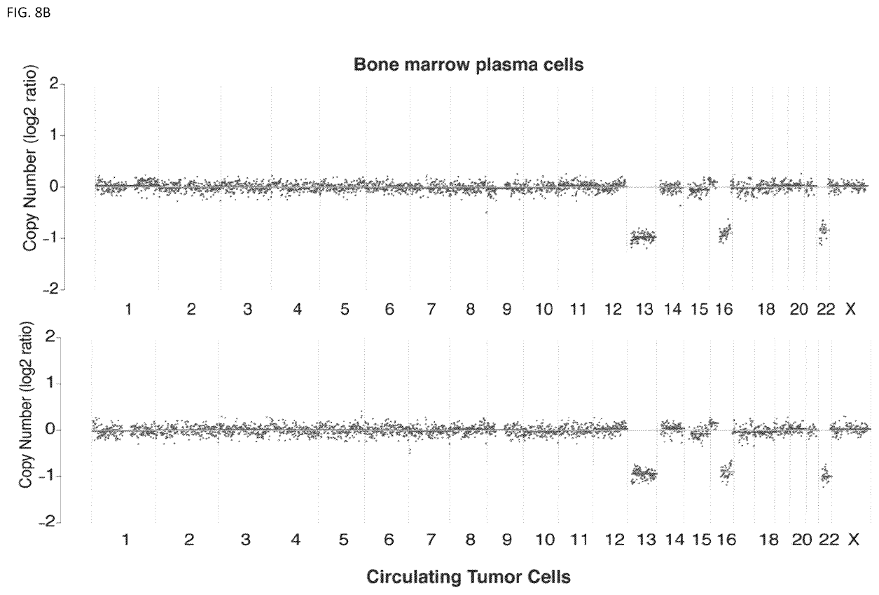

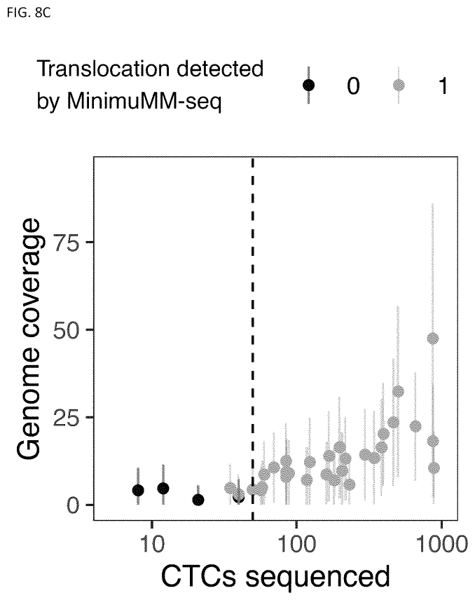

Enumeration of CTCs provides a correlative measure of disease burden, however, molecular characterization can confirm tumor biology and MM-associated genetic alterations. Enriched CTCs that were isolated in a workflow were first characterized to confirm they were malignant cells of good quality and morphology through imaging and ultra-low pass (ULP) WGS and ichorCNA. CTCs were found to be intact and harbor arm-level somatic copy number abnormalities, in concordance with matched BM results [FIGS. 8A and 8B]. Researchers set to establish the minimum number of CTCs that could be recovered and sequenced, without loss of sensitivity for the detection of initiating events of MM, when ground truth of BM fluorescence in situ hybridization (FISH) was known. Quantitative evaluation revealed the lowest limit of detection when 50 CTCs were recovered [FIG. 8C].

With this foundation, the clinical utility of genomic profiling of CTCs using standard WGS without WGA was investigated as an unbiased method to detect abnormalities present in CTCs. Clinical characteristics of all participants are highlighted in Table 3 and sequencing metrics are summarized in Table 4. Overall, a median of 394 CTCs (range, 41 to 75,000) and 11,700 BMPCs (range, 124 to 49,000 for matched sample patients) were isolated and sequenced [FIG. 2A] with paired germline samples as a reference normal comparison. The enrichment approach yielded a median tumor purity of 99% (range, 48 to 100%) and 98% (range, 45 to 100%) for both BMPCs and CTCs, respectively, with quantification by ABSOLUTE (21) [FIG. 2B].

To first illustrate that CTCs could deliver equivalent genomic yield compared to clinical BM testing, matched PB and BM research samples were collected at the same time point of clinic visit from 24 patients (2 MGUS, 15 SMM and 7 MM). All patients with a BM biopsy had routine molecular cytogenetics performed at clinical pathology and FISH analysis recorded [Materials and Methods]. Clinical FISH reported that 11 patients (46%) harbored translocations, 9 patients (37%) had trisomies/copy number changes and 4 patients (17%) had inconclusive results, with BM biopsies showing PC infiltrations in the range of 5% to 80% [Table 5]. Head-to-head comparison of WGS with FISH probes that were tested showed sequencing of CTCs was able to detect 100% (N=44) of abnormalities identified in clinical testing with copy number and structural variant probes [FIG. 2C]. Notably, new clinically reportable high-risk events of IGH-MYC (t(8;14)) translocations and 1q gain were distinguished in 3 patients by WGS, with concordance between CTC and BM compartments, but were not found by FISH (N=3, over 97 successful probes with negative results).

Overall comparison of large-scale events across all patients showed a high concordance of genetic profile between WGS and FISH. Matched CTCs and BMPCs were confirmed to originate from the same clonal expansion through B-cell receptor (BCR) repertoire characterization [Table 6]. Genomic results demonstrate that CTCs are able to faithfully recapitulate all results obtained from ground truth BM, including translocations (t(11;14), t(14;16), t(14;20), t(4;14) and t(8;14)) and copy number abnormalities (chromosomal trisomies, 1q gain, deletion 13q and deletion 16q) [FIG. 2D]. Moreover, WGS of CTCs was able to identify additional genetic events in 20 patients (83%) [FIG. 2D, light gray text]. In particular, 4 SMM patients (CTF001, CTF017, CTF036 and CTF048) had FISH within normal limits or failure due to insufficient plasma cells during clinical testing, however, analysis of CTCs was able to detect and recover cytogenetic abnormality results. In CTF001, MAF translocation t(14;16), 1q gain, and copy loss events were detected on chromosomes 13 and 22. Interestingly, this patient had a BM biopsy with clinical FISH performed again one year after this sampling time point, being successful and reporting t(14;16), deletion 17p, monosomy 13 and 1q gain. In CTF017, a novel IGH-MAFB (t(14;20)) translocation and deletion 13q were detected. In CTF036, trisomies and deletion 13q were found, while in CTF048 trisomies and deletion 6q, 8p were identified. These results highlight that blood-based screening of CTCs can reliably detect alterations to replace BM, even when routine clinical BM fails, to assist with tumor classification.

TABLE 3
Clinical characteristics and sampling
of participants. PB: Peripheral Blood.

| Patient | CTF | | | | IgH and Light | Patient |
|---|---|---|---|---|---|---|
| ID | ID | Age | Gender | Stage | Chain (LC) | Sample |
| 1 | CTF001 | 41 | F | SMM | IgG Kappa | Matched |
| 2 | CTF003 | 51 | F | SMM | IgG Kappa | Matched |
| 3 | CTF002 | 50 | F | SMM | IgG Lambda | Matched |
| 4 | CTF004 | 72 | M | SMM | IgG Lambda | PB |
| 5 | CTF005 | 56 | F | SMM | IgA Lambda | PB |
| 6 | CTF010 | 51 | M | SMM | IgG Kappa | PB |
| 7 | CTF011 | 75 | M | SMM | Biclonal | PB |
| | | | | | IgG Kappa/ | |
| | | | | | IgG Lambda | |
| 8 | CTF012 | 65 | F | SMM | IgG Kappa | Matched |
| 9 | CTF013 | 61 | F | SMM | IgG Lambda | Matched |
| 10 | CTF015 | 78 | F | SMM | IgG Kappa | Matched |
| 11 | CTF016 | 76 | F | SMM | Lambda LC | Matched |
| 12 | CTF017 | 50 | F | SMM | IgG Lambda | Matched |
| 13 | CTF018 | 78 | F | SMM | IgG Kappa | Matched |
| 14 | CTF019 | 61 | M | MM | IgG Kappa | Matched |
| 15 | CTF021 | 63 | M | MM | IgG Kappa | Matched |
| 16 | CTF023 | 84 | M | SMM | IgG Kappa | Matched |
| 17 | CTF022 | 72 | M | MM | IgG Kappa | PB |
| 18 | CTF024 | 73 | F | SMM | IgA Lambda | PB |
| 19 | CTF025 | 77 | F | SMM | IgG Kappa | Matched |
| 20 | CTF026 | 70 | M | MGUS | IgA Kappa | PB |
| 21 | CTF027 | 65 | F | SMM | Lambda LC | PB |
| 22 | CTF028 | 57 | M | MGUS | IgG Kappa | PB |
| 23 | CTF029 | 81 | F | SMM | Biclonal | PB |
| | | | | | IgA/IgG | |
| 24 | CTF030 | 66 | F | SMM | IgA Kappa | PB |
| 25 | CTF031 | 64 | M | SMM | IgG Kappa | PB |
| 26 | CTF032 | 71 | F | SMM | IgG Lambda | PB |
| 27 | CTF033 | 64 | F | SMM | IgG Kappa | PB |
| 28 | CTF034 | 67 | M | MM | IgG Lambda | Matched |
| 29 | CTF035 | 77 | M | SMM | IgG Kappa | PB |
| 30 | CTF036 | 54 | M | SMM | IgG Lambda | Matched |
| 31 | CTF038 | 62 | M | SMM | IgG Kappa | PB |
| 32 | CTF039 | 64 | M | SMM | IgG Lambda | PB |
| 33 | CTF040 | 62 | M | SMM | IgG Lambda | PB |
| 34 | CTF041 | 58 | F | SMM | IgG Kappa | PB |
| 35 | CTF042 | 68 | F | SMM | IgG Kappa | PB |
| 36 | CTF043 | 75 | M | SMM | Kappa LC | PB |
| 37 | CTF044 | 67 | F | MGUS | IgG Lambda | PB |
| 38 | CTF045 | 37 | F | SMM | IgG Kappa | PB |
| 39 | CTF046 | 48 | M | SMM | IgG Kappa | Matched |
| 40 | CTF047 | 56 | M | MM | IgA Lambda | Matched |
| | | | | | LC | |
| 41 | CTF048 | 68 | F | MGUS | IgG Lambda | Matched |
| 42 | CTF049 | 46 | F | MM | IgG Lambda | PB |
| 43 | CTF050 | 74 | M | SMM | Lambda LC | Matched |
| 44 | CTF051 | 51 | F | SMM | IgG Kappa | PB |
| 45 | CTF052 | 76 | F | SMM | IgG Kappa | Matched |
| 46 | CTF053 | 71 | M | MM | IgA Kappa | Matched |
| 47 | CTF054 | 53 | M | MGUS | IgG Kappa | Matched |
| 48 | CTF055 | 60 | M | MM | IgG Lambda | Matched |
| 49 | CTF056 | 47 | F | SMM | IgA Kappa | PB |
| 50 | CTF057 | 68 | M | MM | IgG Kappa | PB |
| 51 | CTF058 | 54 | F | MM | IgG Kappa | Matched |

TABLE 4
WGS coverage and library metrics. Coverage and SNP sensitivity
are calculated as effective post-exclusion of low-mapping
reads, duplicates, overlapping, and low-quality reads.

| Characteristics | Mean (±SD) | Range |
|---|---|---|
| Insert size mode | 314 bp (±37 bp) | 197 to 365 |
| Coverage | 19.1(±11.1) | 3 to 47 |
| % Genome coverage at . . . | | |
| 5X | 92.4% (±13.7%) | 10% to 98.6% |
| 10X | 76.2% (±27.4%) | 1% to 98.3% |
| 15X | 55.4% (±34.8%) | 0% to 97.6% |
| 30X | 18.4% (±28.3%) | 0% to 89.9% |
| Heterozygous SNP Q-score | 14.1 (±3.47) | 3 to 19 |
| % Bases excluded due to . . . | | |
| Low Mapping Quality | 7.3% (±1.3%) | 5% to 11.6% |
| Duplicate Reads | 44.7% (±23.9%) | 0% to 80.1% |
| Unpaired Reads | 0.04% (±0.04%) | 0% to 0.16% |
| Low Base Quality | 1.1% (±0.8%) | 0% to 3.4% |
| Overlapping Reads | 5.0% (±4.5%) | 1% to 17.9% |
| Above Coverage Cap | 0.8% (±0.4%) | 0% to 1.7% |
| Total % Bases excluded | 58.9% (±19.4%) | 10% to 88.9% |

TABLE 5
Clinical BM FISH results and cells recovered for cohort with matched samples.

| | | | IgH and | BM | | | | | | | | | | |
|---|---|---|---|---|---|---|---|---|---|---|---|---|---|---|
| | CTF | | Light | Biopsy | | | | | 1q | 1p | 13q | 16q | 17p | |
| ID | ID | Stage | Chain | PCs % | BMPCs | CTCs | Translocation | Hyperdiploid | dup | del | del | del | del | Notes |
| 1 | CTF001 | SMM | IgG | 20% | 170 | 41 | n/a | n/a | n/a | n/a | n/a | n/a | n/a | Insufficient |
| | | | Kappa | | | | | | | | | | | number of |
| | | | | | | | | | | | | | | plasma cells |
| 2 | CTF003 | SMM | IgG | 15% | 300 | 100 | t(14; 16), | n/a | n/a | n/a | 75% | n/a | n/a | |
| | | | Kappa | | | | 90% | | | | | | | |
| 3 | CTF002 | SMM | IgG | 20% | 124 | 170 | t(14; 16), | n/a | n/a | n/a | n/a | n/a | n/a | |
| | | | Lambda | | | | 70% | | | | | | | |
| 8 | CTF012 | SMM | IgG | 20% | 8300 | 210 | n/a | Trisomy | 40% | n/a | 95% | n/a | n/a | |
| | | | Kappa | | | | | 9, 11, 15 | | | | | | |
| 9 | CTF013 | SMM | IgG | 10% | 30608 | 398 | t(14; 16), | n/a | n/a | n/a | n/a | n/a | n/a | |
| | | | Lambda | | | | 35% | | | | | | | |
| 10 | CTF015 | SMM | IgG | 30% | 8300 | 4135 | t(4; 14), | n/a | n/a | n/a | n/a | n/a | n/a | |
| | | | Kappa | | | | 95% | | | | | | | |
| 11 | CTF016 | SMM | Lambda | 20% | 8300 | 398 | t(11; 14), | n/a | n/a | n/a | n/a | n/a | n/a | |
| | | | LC | | | | 80% | | | | | | | |
| 12 | CTF017 | SMM | IgG | 15% | 4500 | 886 | n/a | n/a | n/a | n/a | n/a | n/a | n/a | Insufficient |
| | | | Lambda | | | | | | | | | | | number of |
| | | | | | | | | | | | | | | plasma cells |
| 13 | CTF018 | SMM | IgG | 40% | 11474 | 2911 | n/a | n/a | 95% | n/a | n/a | n/a | n/a | |
| | | | Kappa | | | | | | | | | | | |
| 14 | CTF019 | MM | IgG | 50% | 947 | 1169 | n/a | Trisomy | 65% | n/a | n/a | n/a | n/a | |
| | | | Kappa | | | | | 9 | | | | | | |
| 15 | CTF021 | MM | IgG | 80% | 12423 | 182 | 14q IGH | Trisomy | n/a | n/a | n/a | n/a | n/a | |
| | | | Kappa | | | | sep, 60% | 3, 9, 11, | | | | | | |
| | | | | | | | | 15 | | | | | | |
| 16 | CTF023 | SMM | IgG | 20% | 23344 | 343 | n/a | Trisomy | n/a | n/a | n/a | n/a | n/a | |
| | | | Kappa | | | | | 3,7,9,11, | | | | | | |
| | | | | | | | | 15(tetra) | | | | | | |
| 19 | CTF025 | SMM | IgG | 30% | 36368 | 198 | n/a | n/a | 70% | n/a | n/a | n/a | n/a | |
| | | | Kappa | | | | | | | | | | | |
| 28 | CTF034 | MM | IgG | 80% | 16880 | 654 | n/a | Trisomy | n/a | n/a | n/a | n/a | n/a | |
| | | | Lambda | | | | | 3, 9, 15 | | | | | | |
| 30 | CTF036 | SMM | IgG | 20% | 49395 | 286 | n/a | n/a | n/a | n/a | n/a | n/a | n/a | Insufficient |
| | | | Lambda | | | | | | | | | | | number of |
| | | | | | | | | | | | | | | plasma cells |
| 39 | CTF046 | SMM | IgG | 30% | 2051 | 523 | n/a | n/a | n/a | n/a | n/a | n/a | n/a | Insufficient |
| | | | Kappa | | | | | | | | | | | number of |
| | | | | | | | | | | | | | | plasma cells |
| 40 | CTF047 | MM | IgA | 80% | 16700 | 32071 | t(14; 16), | n/a | n/a | n/a | n/a | n/a | n/a | |
| | | | Lambda | | | | 100% | | | | | | | |
| | | | LC | | | | | | | | | | | |
| 41 | CTF048 | MGUS | IgG | 10% | 9906 | 266 | n/a | n/a | n/a | n/a | n/a | n/a | n/a | |
| | | | Lambda | | | | | | | | | | | |
| 43 | CTF050 | SMM | Lambda | 20% | 4343 | 227 | n/a | n/a | 72% | n/a | n/a | n/a | n/a | |
| | | | LC | | | | | | | | | | | |
| 45 | CTF052 | SMM | IgG | 50% | 8300 | 168 | t(11; 14), | n/a | n/a | n/a | 88% | n/a | n/a | |
| | | | Kappa | | | | 78% | | | | | | | |
| 46 | CTF053 | MM | IgA | 50% | 8300 | 124 | t(4; 14), | n/a | n/a | n/a | n/a | n/a | n/a | |
| | | | Kappa | | | | 100% | | | | | | | |
| 47 | CTF054 | MGUS | IgG | 5% | 17243 | 70 | t(14; 20), | n/a | n/a | n/a | n/a | n/a | n/a | |
| | | | Kappa | | | | 72% | | | | | | | |
| 48 | CTF055 | MM | IgG | 70% | 20783 | 871 | t(4; 14), | Trisomy | 76% | n/a | n/a | n/a | n/a | |
| | | | Lambda | | | | 77% | 3, 9, 15 | | | | | | |
| 51 | CTF058 | MM | IgG | 80% | 8300 | 3061 | t(14; 16), | n/a | 86% | n/a | n/a | n/a | n/a | |
| | | | Kappa | | | | 92% | | | | | | | |

TABLE 6
Comparison of BCR sequence with VDJ and CDR3 exact match between BMPCs and
CTCs obtained by mixcr algorithm. Rows are marked by detection in both CTCs and BMPCs
(italicized). Bolded rows depict major clone found in BMPCs but not reconstructed in CTC.
Allele fraction is given as a percentage of all BCR detected. No IGH BCR was reconstructed for
matched patient CTF016 in either CTCs or BMPCs.

| | | | | | Clone | Clone |
|---|---|---|---|---|---|---|
| | | | | | Allele | Allele |
| Parti- | | | | CDR3 Aminoacid sequence | Fraction | Fraction |
| cipant | V_segment | D_segment | J_segment | (SEQ ID NOs) | BMPCs | CTCs |
| CTF001 | IGHV3-11*00 | IGHD3-22*00 | IGHJ6*00 | CTRGHYYDSSGYSLIKGYNYYYYLDVW (5) | 100% | 80% |
| CTF001 | IGHV4-39*00 | IGHD3-10*00 | IGHJ5*00 | CARQGVLGFGVSNWFDPW (6) | 0% | 20% |
| CTF002 | IGHV3-21*00 | IGHD5-12*00 | IGHJ3*00 | CARDLSKLAEAFDIW (7) | 71% | 80% |
| CTF002 | IGHV3-13*00 | IGHD3-22*00 | IGHJ4*00 | CATRTMIVV_LLPTPPDYW (8, 44) | 29% | 20% |
| CTF003 | IGHV1-24*00 | IGHD6-13*00 | IGHJ6*00 | CATEISPAIPPLGYGLGVW (9) | 42% | 57% |
| CTF003 | IGHV3-16*00 | IGHD3-22*00 | IGHJ4*00 | CVRKRVVLL**SGYYWGFDYW (11, 12) | 33% | 43% |
| CTF003 | IGHV3-35*00 | IGHD3-22*00 | IGHJ4*00 | CVRKRVVLL**SGYYWGFDYW (11, 12) | 25% | 0% |
| CTF012 | IGHV1-46*00 | IGHD4-17*00 | IGHJ6*00 | CARMEASYAPTHNFYGLDVW (13) | 94% | 100% |
| CTF012 | IGHV4-28*00 | IGHD4-17*00 | IGHJ6*00 | CARMEASYAPTHNFYGLDVW (13) | 6% | 0% |
| CTF013 | IGHV4-59*00 | IGHD3-16*00 | IGHJ4*00 | CARDQGGPFDHW (14) | 87% | 100% |
| CTF013 | IGHV3-15*00 | IGHD3-10*00 | IGHJ4*00 | CTLDYFGSGSNYNKYW (15) | 7% | 0% |
| CTF013 | IGHV4-59*00 | IGHD3-16*00 | IGHJ5*00 | CARDQGGPFDHW (14) | 7% | 0% |
| CTF015 | IGHV1-3*00 | IGHD3-16*00 | IGHJ2*00 | CATLPDDYGVDYGYWYFDLW (16) | 95% | 94% |
| CTF015 | IGHV1-67*00 | IGHD3-16*00 | IGHJ2*00 | CATLPDDYGVDYGYWYFDLW (16) | 5% | 0% |
| CTF015 | IGHV1-46*00 | IGHD3-16*00 | IGHJ2*00 | CATLPDDYGVDYGYWYFDLW (16) | 0% | 6% |
| CTF017 | IGHV3-13*00 | IGHD2-2*00 | IGHJ5*00 | CARGL***QL_MRCNWFDPW (17, 18) | 33% | 100% |
| CTF017 | IGHV1-18*00 | IGHD3-3*00 | IGHJ5*00 | CARGVRITIFGVAGGWTSPEDGEKDGLD | 11% | 0% |
| | | | | PW (19) | | |
| CTF017 | IGHV4-39*00 | IGHD3-22*00 | IGHJ4*00 | CARHRLATYYYESSGYYFDYW (20) | 11% | 0% |
| CTF017 | IGHV4-34*00 | IGHD6-19*00 | IGHJ5*00 | CAGWGRTLPFLSNWFDPW (21) | 11% | 0% |
| CTF017 | IGHV1-18*00 | IGHD5-24*00 | IGHJ4*00 | CARDWDMATIRGGGDYW (22) | 11% | 0% |
| CTF017 | IGHV3-15*00 | IGHD2-8*00 | IGHJ4*00 | CTTQIYCTNGVCADYW (23) | 11% | 0% |
| CTF017 | IGHV4-39*00 | IGHD6-19*00 | IGHJ4*00 | CVLPLVGTVYVGYW (24) | 11% | 0% |
| CTF018 | IGHV4-31*00 | IGHD1-7*00 | IGHJ5*00 | CARDWNYGTNSFWFDPW (25) | 100% | 95% |
| CTF018 | IGHV6-1*00 | IGHD1-7*00 | IGHJ5*00 | CARDWNYGTNSFWFDPW (25) | 0% | 5% |
| CTF019 | IGHV2-70*00 | IGHD3-10*00 | IGHJ4*00 | CARIRDYYASGAHDFW (26) | 100% | 100% |
| CTF021 | IGHV3-21*00 | IGHD3-3*00 | IGHJ6*00 | CARFGGRDFWSGYSGYYHYGMDVW (27) | 100% | 100% |
| CTF023 | IGHV2-5*00 | IGHD3-10*00 | IGHJ4*00 | CVHRQGRTLLRGAMSPYFDFW (28) | 100% | 0% |
| CTF025 | IGHV3-43*00 | IGHD4-23*00 | IGHJ1*00 | CVKGDYGRNPGHFEYW (29) | 100% | 100% |
| CTF034 | IGHV3-33*00 | IGHD4-17*00 | IGHJ4*00 | CARDCDS**H_GVTTL*FDYW (30, 31, | 67% | 72% |
| | | | | 32) | | |
| CTF034 | IGHV3-48*00 | IGHD3-9*00 | IGHJ3*00 | CARILSDFGDHDRRDAFDVW (33) | 33% | 22% |
| CTF034 | IGHV3-21*00 | IGHD3-9*00 | IGHJ3*00 | CARILSDFGDHDRRDAFDVW (33) | 0% | 6% |
| CTF036 | IGHV1-3*00 | IGHD6-19*00 | IGHJ4*00 | CASEIVGWAFDYW (34) | 90% | 83% |
| CTF036 | IGHV1-67*00 | IGHD6-19*00 | IGHJ4*00 | CASEIVGWAFDYW (34) | 10% | 0% |
| CTF036 | IGHV1-46*00 | IGHD6-19*00 | IGHJ4*00 | CASEIVGWAFDYW (34) | 0% | 17% |
| CTF046 | IGHV1-24*00 | IGHD3-16*00 | IGHJ4*00 | CATSALGQVDNW (35) | 100% | 67% |
| CTF046 | IGHV3-53*00 | IGHD6-13*00 | IGHJ5*00 | CARSYSSSLRGDWFDPW (43) | 0% | 33% |
| CTF047 | IGHV3-49*00 | IGHD5-12*00 | IGHJ4*00 | CSRDGLGIVATGDVDSGGLDRW (36) | 100% | 100% |
| CTF048 | IGHV3-9*00 | IGHD3-22*00 | IGHJ6*00 | CVKDLSNGYYSLDANHYFGMDVW (37) | 100% | 100% |
| CTF052 | IGHV1-69*00 | IGHD2-21*00 | IGHJ6*00 | CVRGEDEVTAIDYYYFGMDVW (38) | 100% | 0% |
| CTF053 | IGHV1-18*00 | IGHD4-17*00 | IGHJ4*00 | CAREGDDYDDYNYLDYW (39) | 100% | 100% |
| CTF054 | IGHV5-51*00 | IGHD3-10*00 | IGHJ4*00 | CARRFGGSYFDYW (40) | 100% | 100% |
| CTF055 | IGHV3-30*00 | IGHD6-13*00 | IGHJ4*00 | CAKGIYSSSFTRARDSW (41) | 100% | 100% |
| CTF058 | IGHV4-31*00 | IGHD6-13*00 | IGHJ5*00 | CARGLSWEVPAAAWFDPW (42) | 100% | 100% |

### Example 3: MimimumMM-seq Enables Genomic Profiling of CTCs for Unbiased WGS-Based Molecular Analyses

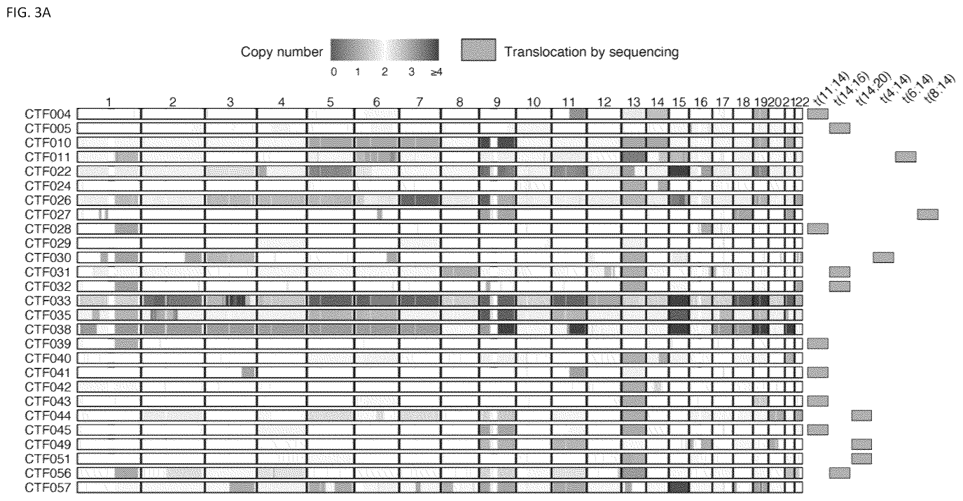

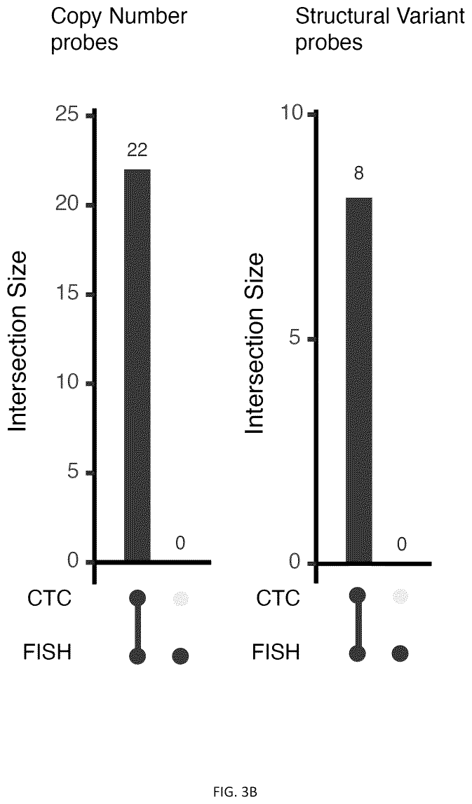

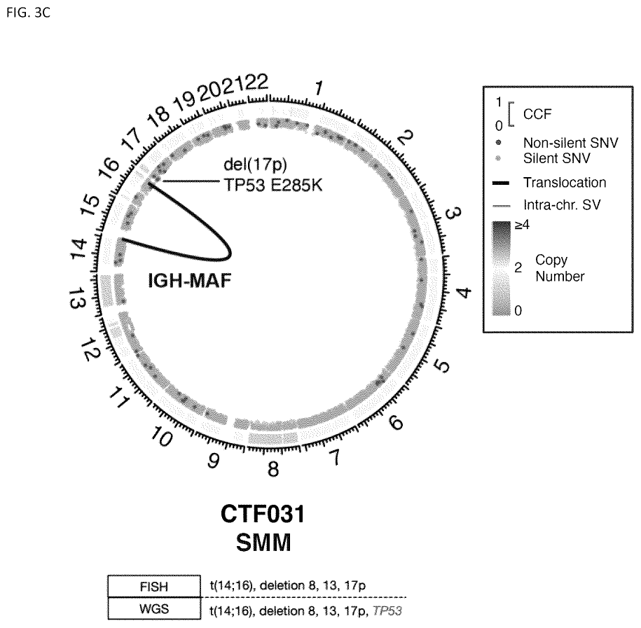

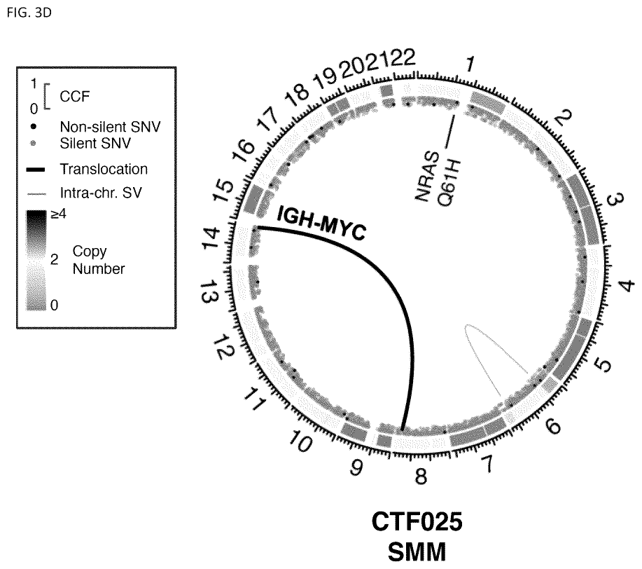

As characterization of CTCs proved feasible, the utility of PB sampling and WGS of CTCs as a diagnostic tool for minimally invasive detection of molecular events in patients in the absence of a BM reference was explored. To extend the assessment and demonstrate the applicability of MinimuMM-seq as a clinical tool, a validation cohort was collected of prospective PB-only samples from 27 patients (3 MGUS, 22 SMM and 2 MM). Comprehensive detection of chromosomal abnormalities was demonstrated, with the ability to detect key translocations and copy number variants of MM across all patients using CTCs [FIG. 3A]. Sixteen patients (59%) harbored translocations, including frequent and infrequent translocations such as t(11;14), t(4;14), t(14;16), t(14;20), t(6;14) and t(8;14). Eleven patients (41%) showed trisomies and copy number changes including 1q gain, 1p deletion, odd chromosomal trisomies and deletion 13q. Due to the asymptomatic nature of precursor patients, all were under careful clinical observation and most patients had a BM biopsy and FISH analyses performed at one time point during their ongoing monitoring, which was used as a known reference [Table 7]. Evaluation of WGS of CTCs, in comparison to FISH probes that were tested on BM, firmly showed CTCs were able to detect 100% of abnormalities identified in clinical testing [FIG. 3B]. In 6 patients (CTF022, 026, 027, 029, 040 and 044) no clinical FISH data was available (normal or insufficient cells), however, genomic characterization of CTCs revealed the presence of trisomies, t(14;16), rare IGH-MAFA translocation (t(8;14)), deletion 1p and 13q, which are important cytogenetic abnormalities with a predictive value for clinical understanding of a premalignant patient.

As such, two key examples of the application are highlighted in blood sampling of SMM finding high-risk events. Participant CTF031 clinical testing showed t(14;16), deletion of 8q, 13q and 17p. Additionally, sensitivity of WGS was found to layer in mutations with the detection of single nucleotide variants (SNV) in TP53, inducing a likely pathogenic variant p.E285K (22). Notably, this result confers a biallelic double hit event, which is a high-risk category for MM patients that conventional testing would not be able to uncover [FIG. 3C]. From clinical reports, participant CTF025 harbored an IGH separation, 1q gain and trisomies. WGS of CTCs revealed the unknown translocation with chromosome 14 to be IGH-MYC (t(8;14)), with additional trisomies and NRAS hotspot mutation p.Q61H. Studies have shown MYC-IGH translocations as high-risk events, compared to non-IgHMYC translocations, indicating an increased potential for progression from SMM to MM within 2 years (23,24) [FIG. 3D]. Taken together, these results exemplify the resolution afforded by sequencing-based methods to characterize tumor biology for improved clinical decisions on early intervention strategies.

TABLE 7
Clinical BM FISH results of peripheral blood only cohort and CTCs recovered

| | | | | BM | | | | | | | | | |
|---|---|---|---|---|---|---|---|---|---|---|---|---|---|
| | | | IgH and | Biopsy | | | | 1q | 1p | 13q | 16q | 17p | |
| ID | CTF ID | Stage | Light Chain | PCs % | CMMCs | Translocation | Hyperdiploid | dup | del | del | del | del | Notes |
| 4 | CTF004 | SMM | IgG Lambda | 10% | 60 | t(11; 14), | n/a | n/a | n/a | n/a | n/a | n/a | |
| | | | | | | 80% | | | | | | | |
| 5 | CTF005 | SMM | IgA Lambda | 10% | 57 | t(14; 16) | n/a | n/a | n/a | n/a | n/a | n/a | |
| 6 | CTF010 | SMM | IgG Kappa | 20% | 402 | 14q IGH | n/a | n/a | n/a | 85% | n/a | n/a | Trisomy |
| | | | | | | sep, 50% | | | | | | | 6p (75%) |
| 7 | CTF011 | SMM | Biclonal | 15% | 297 | n/a | n/a | ✓ | n/a | 85% | n/a | n/a | |
| | | | IgG Kappa/ | | | | | | | | | | |
| | | | IgG Lambda | | | | | | | | | | |
| 19 | CTF022 | SMM | IgG Kappa | 20% | 206 | n/a | Trisomy | n/a | n/a | ✓ | n/a | n/a | |
| | | | | | | | 5, 9, 15 | | | | | | |
| 20 | CTF024 | SMM | IgA Lambda | 13% | 217 | n/a | n/a | n/a | n/a | ✓ | n/a | n/a | |
| 22 | CTF026 | MGUS | IgA Kappa | 10% | 81 | n/a | n/a | n/a | n/a | n/a | n/a | n/a | No BM |
| | | | | | | | | | | | | | FISH ever |
| | | | | | | | | | | | | | performed |
| 23 | CTF027 | SMM | Lambda LC | 10% | 4979 | n/a | n/a | ✓ | n/a | n/a | n/a | n/a | |
| 24 | CTF028 | MGUS | IgG Kappa | 10% | 162 | n/a | n/a | n/a | n/a | n/a | n/a | n/a | |
| 25 | CTF029 | SMM | Biclonal | 20% | 58 | t(11; 14), | n/a | n/a | n/a | n/a | n/a | n/a | |
| | | | IgA/IgG | | | 100% | | | | | | | |
| 26 | CTF030 | SMM | Lambda | 20% | 503 | t(4; 14) | Trisomy 3 | ✓ | n/a | ✓ | n/a | n/a | |
| | | | high | | | | | | | | | | |
| 27 | CTF031 | SMM | IgG Kappa | 30% | 41500 | t(14; 16), | n/a | n/a | n/a | 85% | n/a | 70% | 8q del, |
| | | | | | | 85% | | | | | | | 95% |
| 28 | CTF032 | SMM | IgG Lambda | 50% | 9372 | t(14; 16), | n/a | 75% | n/a | 90% | n/a | n/a | |
| | | | | | | 70% | | | | | | | |
| 29 | CTF033 | SMM | IgG Kappa | 20% | 755 | 14q IGH | Trisomy | n/a | n/a | n/a | n/a | n/a | |
| | | | | | | sep, 40% | 3, 7, 9, | | | | | | |
| | | | | | | | 11, 15 | | | | | | |
| 31 | CTF035 | SMM | IgG Kappa | 70% | 184 | n/a | Trisomy | 90% | n/a | n/a | n/a | n/a | |
| | | | | | | | 9, 11, 15 | | | | | | |
| 33 | CTF038 | SMM | IgG Kappa | 20% | 523 | n/a | n/a | 76% | n/a | n/a | n/a | n/a | |
| 34 | CTF039 | SMM | IgG Lambda | 20% | 232 | t(11; 14) | n/a | n/a | ✓ | n/a | n/a | n/a | |
| 35 | CTF040 | SMM | IgG Lambda | 20% | 171 | n/a | n/a | n/a | n/a | n/a | n/a | n/a | |
| 36 | CTF041 | SMM | IgG Kappa | 10% | 386 | t(11; 14) | i | n/a | n/a | n/a | n/a | n/a | |
| 37 | CTF042 | SMM | IgG Kappa | 20% | 230 | 14q IGH | n/a | n/a | n/a | ✓ | n/a | n/a | |
| | | | | | | sep | | | | | | | |
| 38 | CTF043 | SMM | Kappa LC | 30% | 218 | t(11; 14), | n/a | n/a | n/a | 100% | n/a | n/a | |
| | | | | | | 100% | | | | | | | |
| 39 | CTF044 | MGUS | IgG Lambda | n/a | 872 | n/a | n/a | n/a | n/a | n/a | n/a | n/a | |

### Example 4: Longitudinal Liquid Biopsy and Serial WGS of CTCs Reveal Clonal Architecture and Evolutionary History

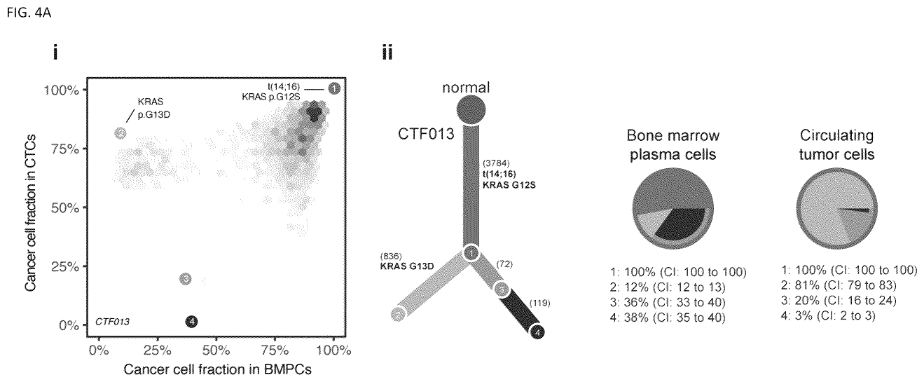

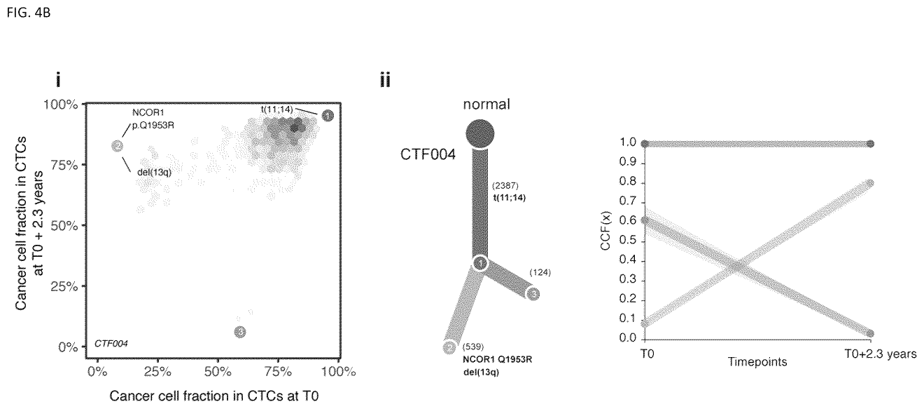

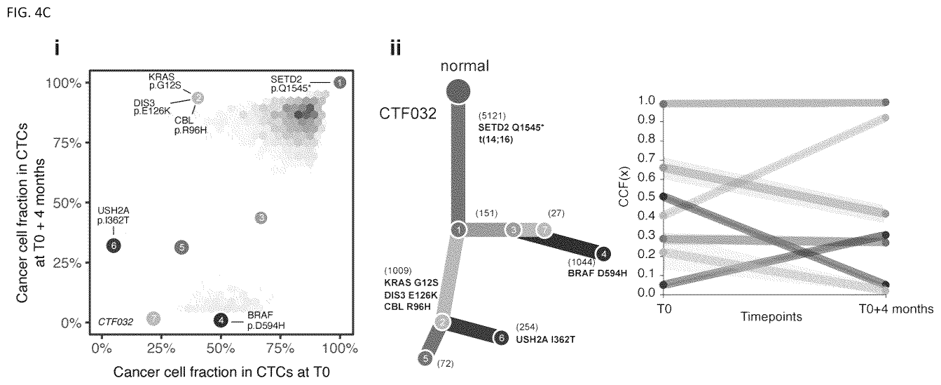

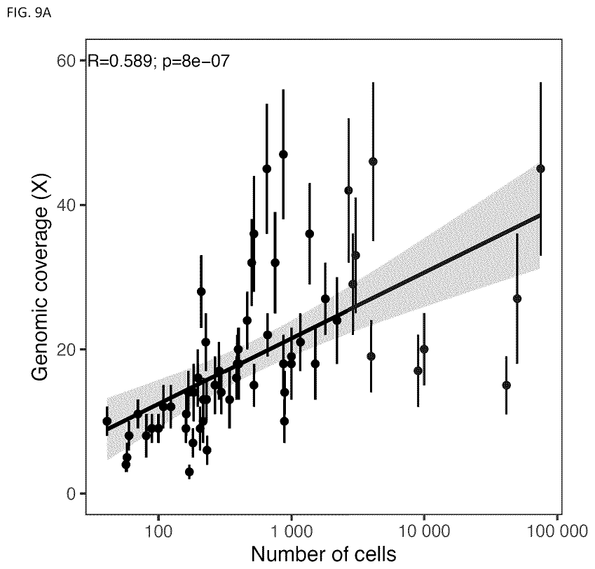

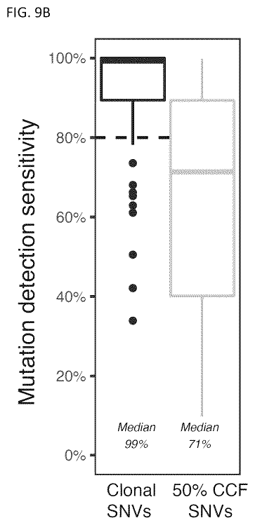

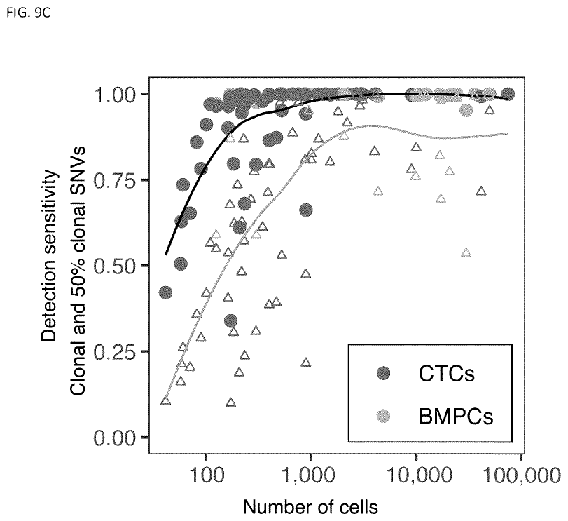

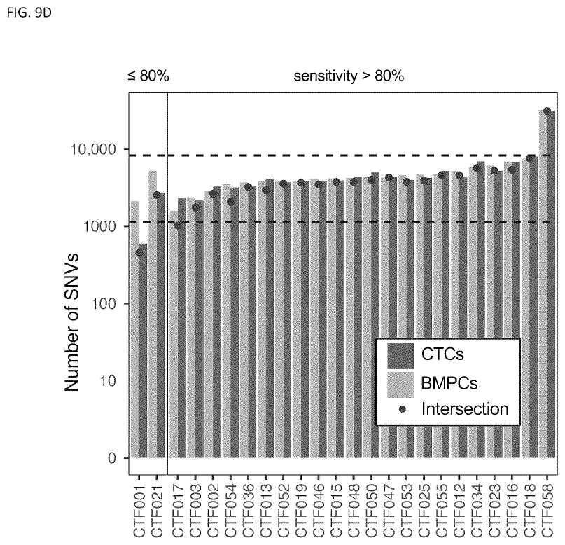

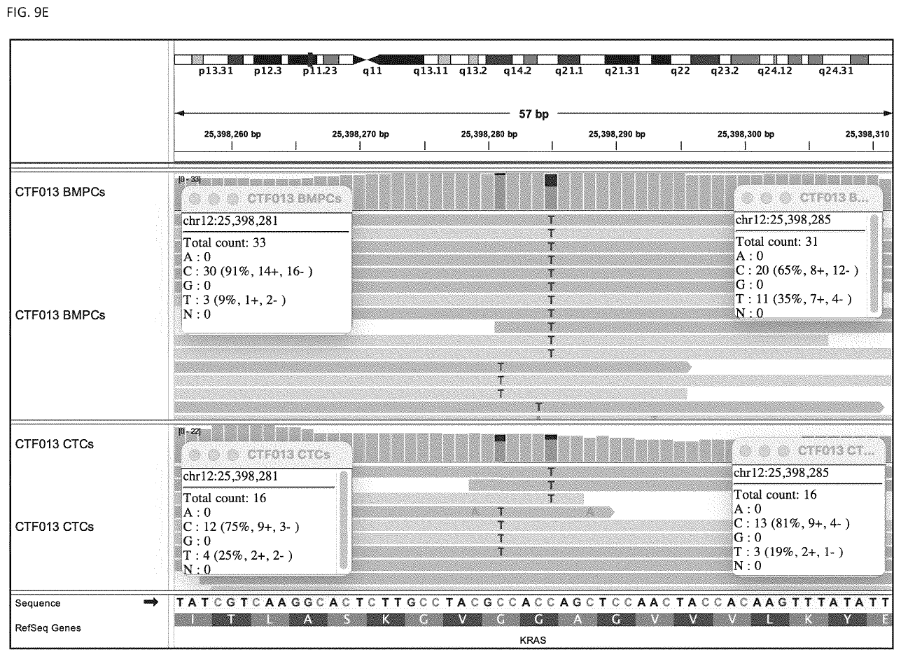

As peripheral blood continuously circulates, liquid biopsy sampling may be affected by the phenomena of spatial and temporal heterogeneity in CTC burden. In patients with matched samples, it was investigated whether the major BM clone possessed the potential to extravasate and circulate, or whether preferential circulation of subclones occurred. Studies showed that large-scale copy number and IgH translocations are shared between both BM and the CTCs [FIG. 2D]. The sensitivity was calculated to detect point mutations in CTCs and BM given purity, ploidy, and sequencing depth of coverage [FIG. 9A-C]. Sensitivity was 99% (IQR, 92% to 100%) for clonal events (cancer cell fraction, CCF=1), and 75% (IQR, 43% to 94%) for mutations occurring ≥50% CCF. Two patients with matched BMPCs fell below an average detection power of 80% for clonal events due to their low number of captured CTCs [FIG. 9D]. Total mutational load across all samples was in the range of 1,000 to 9,000 SNVs and short insertions or deletions (indels), in line with the expected burden from previous genome studies of MM PCs(2,3,25) [FIG. 9D]. At the genome-wide scale, mutations were more likely to be shared between both compartments [FIG. 9D], indicating the shared clonal history of BMPCs and the CTCs. In participant CTF013, this is exemplified by a high mutation density at the upper diagonal (100% CCF), with t(14;16) and the first KRAS hotspot mutant (p.G12S) cluster [FIG. 4Ai]. A second high-density cluster was found with the second KRAS hotspot (p.G13D), having a vertical shift from the diagonal suggesting it is preferentially found in the peripheral blood compartment. Next, used PhylogicNDT (26) was used to cluster mutations and copy number abnormalities into branching subclones and found that KRAS p.G12S is indeed predicted to be a shared clonal event (light gray), while KRAS p.G13D belongs to a subclonal branch (dark gray) [FIG. 4Aii, FIG. 9E]. This suggests that KRAS p.G13D confers an additional fitness advantage to tumor cells, and indeed is more common than the p.G12S mutation in the MM disease setting (27).

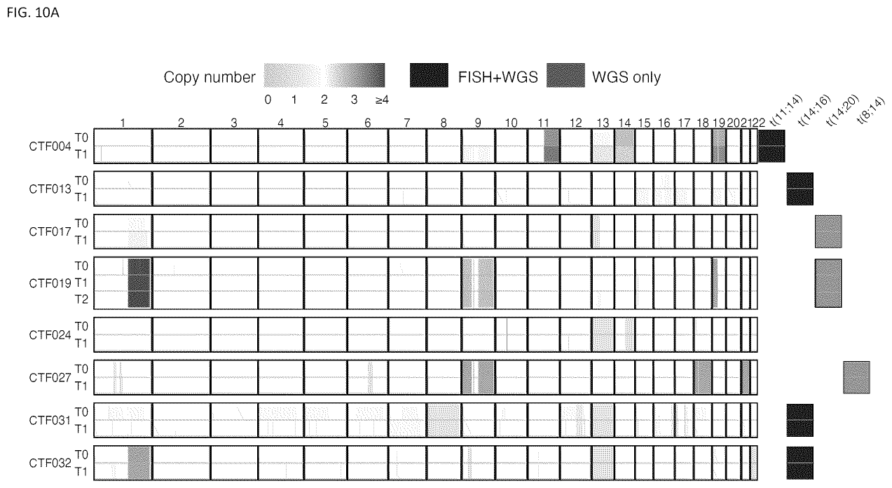

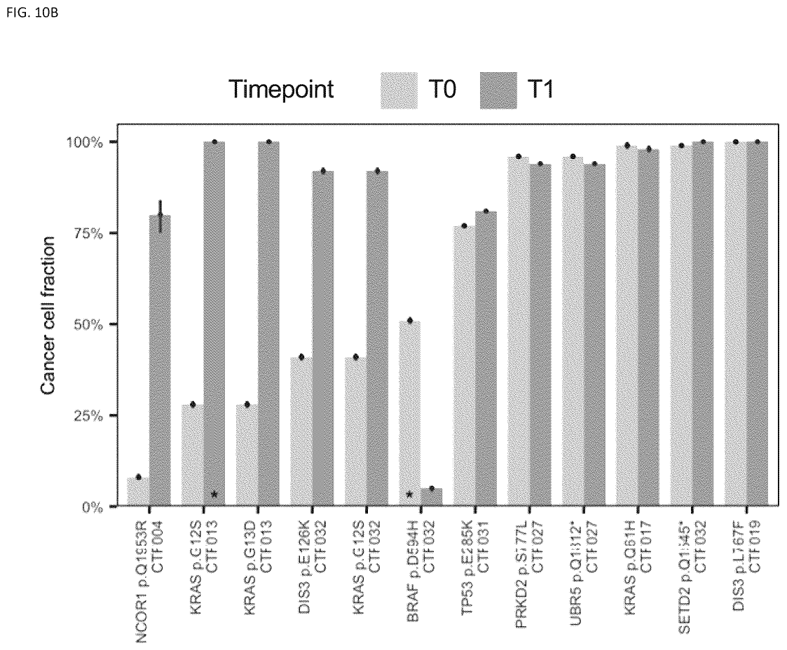

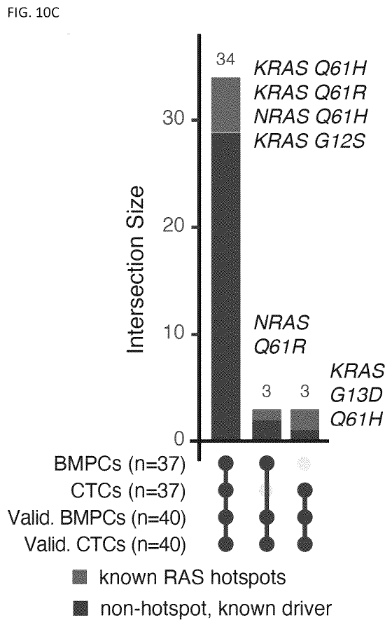

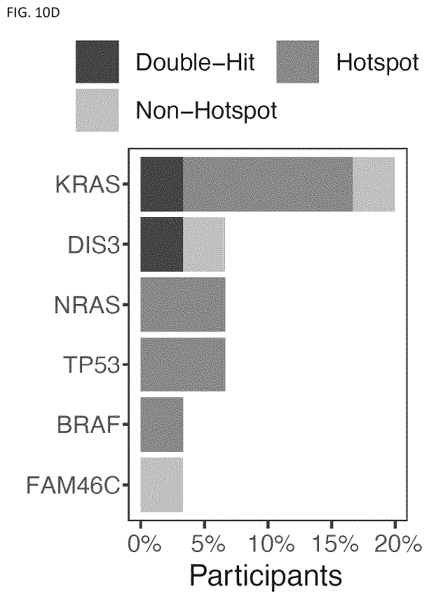

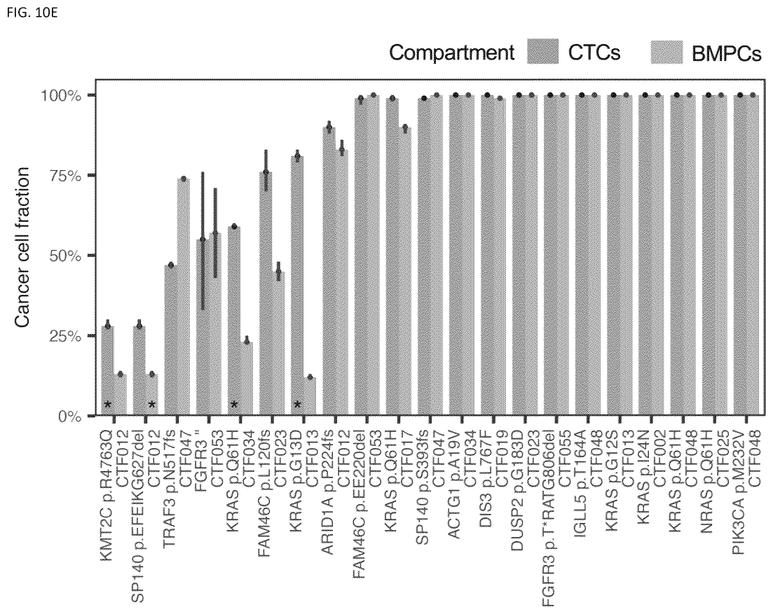

As clonal complexity is not limited to spatial heterogeneity, but also temporal changes over time, serial samples were analyzed from 8 cases of SMM (CTF004, 013, 017, 019, 024, 027, 031, 032), of which 7 patients remained under careful clinical observation without treatment (time range, 2 to 33 months). In all cases stability of the major clone was validated, with clonal evolution and shifting dynamics of CTC subclones over time [FIG. 10A]. Expansion of subclones harboring MM driver mutations such as KRAS (both G12 and G13 hotspots) and DIS3 was observed, indicating their potential to confer selective advantage for clonal fitness and independence for circulation [FIG. 10B]. In an example case of CTF004, longitudinal sampling over 28 months demonstrated the reliability of the approach for diagnostic sampling based on liquid biopsy with stability of mutation presence with clonal t(11;14) and gain of 11q validated at both time points [FIG. 4Bi]. Additionally, over time, an emergent subclone with increased clonal fitness appeared harboring an additional deletion of 13q, growing from CCF of 8% (CI: [8%, 9%]) at T0, to CCF of 80% at T1 (CI: [75%, 84%]), while simultaneous extinction of orange subclone was observed [FIG. 4Bii]. These data demonstrate the use of CTCs to successfully track both acquisition of mutations and clonal dynamics of MM disease over time in a minimally invasive manner.

Of the 8 patients with serial blood collection, one SMM (CTF032) received early interventional treatment following first blood collection and CTC sequencing. After 4 months of therapy, the patient achieved a partial response with serum M-spike concentration decreased by 70% (1.16 to 0.37 g/dL) and CTC counts decreased by 86% (12,769 to 1,803), however serial CTC profiling was able to give a readout of clonal tiding (i.e., switching of clones), with potential drug-related dynamics evident in real-time, described here as an example. Within this period, abundance of myeloma cells with BRAF p.D594H kinase-dead mutation (28) decreased from 51% (CI: [50%, 52%]) to 5% (CI: [4%, 5%]) [FIG. 4Ci-ii]. Simultaneously, a minor clone present at baseline with oncogenic KRAS p.G12S activating hotspot mutation showed a relative CCF increase from 41% (CI: [40%, 42%]) to 92% (CI: [91%, 93%]). The patient progressed to overt multiple myeloma after end of treatment, while PhylogicNDT modeling predicted that clones bearing KRAS p.G12S were selected, in combination with other mutants that are part of the dark gray branch also bearing USH2A p.I362T, a gene recurrently mutated in POEMS syndrome (29). Selection of the new fittest clone under treatment is revealed by a minimally-invasive method before any potential resistance is clinically observed.

### Example 5: Genomic Profiling of CTCs can Replace Clinical BM FISH for Risk Classification of Patients

In the genomics era, implementation of clinical sequencing to improve stratification of patients based on genomic biomarkers has high potential, going beyond the use of correlative clinical markers alone in models such as the IMWG 20-2-20 for SMM (20) and International Staging System (ISS) for MM (30). Detection of chromosomal abnormalities has been implemented to extend both systems (which became the four-factor model and the Revised ISS (31), respectively). The ability to use genomic profiling of CTCs for this stratification would provide a minimally invasive and repeatable solution to refine patient classification.

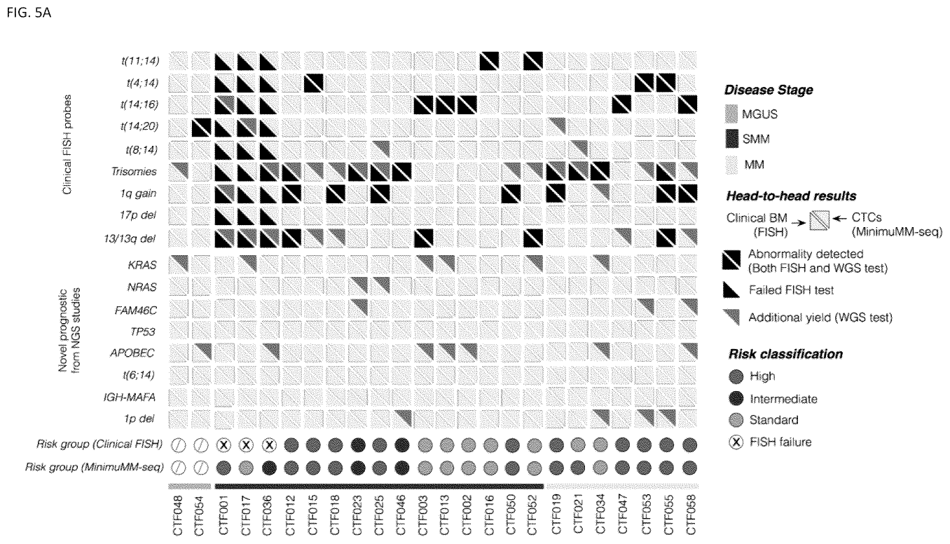

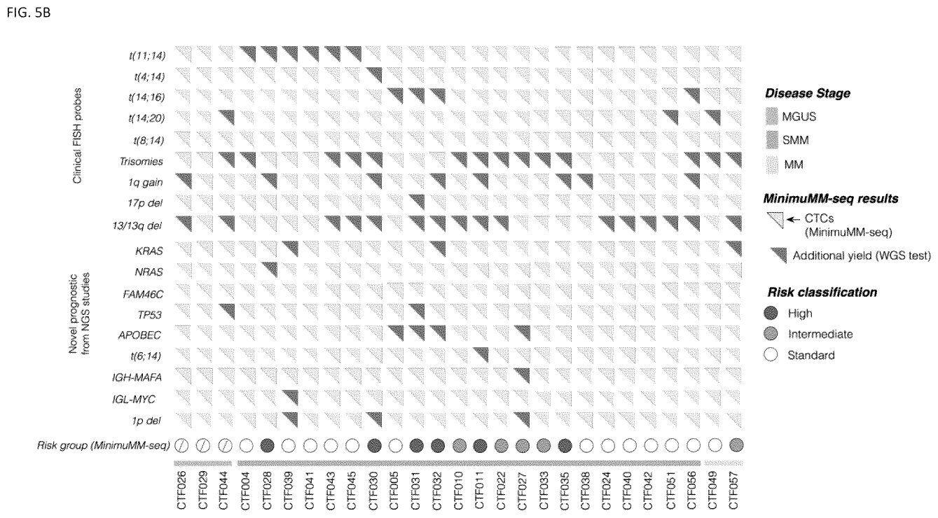

Sequencing of CTCs was able to detect all reported high-risk prognostic factors including t(4;14), t(14;16) 1q gain, del(13q), del(17)p. Risk stratification carried out with WGS of CTCs, using available cytogenetic-based risk models (31,32), showed matched patients would have been assigned to the correct risk group when BM FISH was successful. Additionally, the recovery of FISH failures (due to insufficient cells for testing), or additional diagnostic yield enabled attribution of clinical risk [FIG. 5A]. Moreover, detection of novel driver mutations (KRAS, NRAS, FAM46C), MYC rearrangements, and signatures (APOBEC activity) by NGS are also shown and could help in predicting which patients are at high risk of transformation. Similarly, in SMM patients where only CTCs were sampled, diagnostic yield was obtained from MinimuMM-seq allowing for the assignment of risk classification [FIG. 5B]. In 5 cases of MGUS (CTF026, CTF029, CTF044, CTF048, and CTF054), CTCs could also be used to obtain a genetic result. Usually, clinical BM is not performed at this very early stage due to low disease burden, as such, minimally invasive blood testing may be a rapid solution to overcome limitations of both sampling and patient discomfort for a closer observation of MGUS patients. No cytogenetic risk association models for MGUS are present, but observed trisomies, t(14;20), 1q gain and deletion of 13q were observed, potentially detailing high-risk patients. Taken together, these data show that genomic assessment of CTCs provides demonstrable results that match BM sampling, and the potential of the power of their clinical application for cytogenetic assessment of patients.

### Example 6: Circulating Tumor Cells (CTCs) Sequencing Enables Cohort-Level Genomic Study of Mutational Signatures and Complex Structural Events

As whole-genome sequencing (WGS) is a next generation sequencing (NGS) technology that can sequence the entire genome, its application in CTCs may also provide more comprehensive mutation data across all scales without prior knowledge. Mutation and structural variants counts are summarized in Tables 8 and 9. Mutations in the RAS-MAPK pathway are the most prevalent and significantly mutated drivers in multiple myeloma (MM), with KRAS and NRAS mutated in 21% and 19.5% of MM patients (1,5,6), respectively. Clonal and subclonal MAPK mutations of KRAS and NRAS were found in 12 patients out of 51 (24%), including hotspot mutations of G12, G13 and Q61 [FIG. 10C]. Mutations were also found in 6 driver genes of MM (BRAF, DIS3, FAM46C, KRAS, NRAS and TP53) in 17 patients (33%) [FIG. 10D], with concordance between CTCs and matched BM. All mutations found in CTCs in driver genes were validated in the BM compartment by specifically looking for sequencing reads that support the mutant allele [FIG. 10D-E].

TABLE 8
Enumeration of single nucleotide variants and short
insertions and deletions discovered from WGS of CTCs.

| Single Nucleotide | | Median per | Median clonal per | |
|---|---|---|---|---|
| Variants | | participant | participant | Median subclonal per |
| and short indels | Total | (range) | (range) | participant (range) |
| Total | 257423 | 3676 (314 to 30929) | 2413 (281 to 8920) | 1248 (31 to 22009) |

By variant class

| | | | | |
|---|---|---|---|---|
| 3′UTR | 1638 | 20 (2 to 262) | 13 (2 to 80) | 7 (0 to 182) |
| S′Flank | 10090 | 130 (11 to 1472) | 84 (10 to 396) | 43 (1 to 1076) |
| S′UTR | 650 | 7 (1 to 143) | 4 (0 to 31) | 3 (0 to 112) |
| Could not be determined | 4 | 1 (0 to 1) | 1 (0 to 1) | 0 (0 to 1) |
| De novo start in frame | 6 | 1 (0 to 1) | 1 (0 to 1) | 0 (0 to 1) |
| De novo start out of frame | 8 | 1 (0 to 1) | 0 (0 to 1) | 0 (0 to 1) |
| Frameshift deletion | 52 | 2 (0 to 5) | 1 (0 to 2) | 1 (0 to 3) |
| Frameshift insertion | 12 | 1 (0 to 1) | 1 (0 to 1) | 0 (0 to 1) |
| Intergenic region | 130641 | 1946 (179 to 14476) | 1285 (159 to 4338) | 650 (19 to 10138) |
| Inframe deletion | 18 | 1 (0 to 2) | 0 (0 to 2) | 1 (0 to 1) |
| Inframe insertion | 3 | 1 (0 to 1) | 1 (0 to 1) | 0 (0 to 1) |
| Intron | 88794 | 1202 (96 to 11712) | 777 (87 to 3253) | 417 (9 to 8459) |
| Missense mutation | 1530 | 19 (2 to 242) | 10 (1 to 55) | 8 (0 to 187) |
| Nonsense mutation | 95 | 1 (0 to 24) | 1 (0 to 5) | 1 (0 to 19) |
| Nonstop mutation | 1 | 1 (0 to 1) | 1 (1 to 1) | 0 (0 to 0) |
| RNA | 23136 | 342 (24 to 2482) | 218 (21 to 735) | 116 (2 to 1747) |
| Silent | 622 | 7 (0 to 100) | 5 (0 to 23) | 2 (0 to 77) |
| Splice site | 117 | 2 (0 to 11) | 1 (0 to 6) | 1 (0 to 7) |
| Start codon SNP | 5 | 1 (0 to 1) | 0 (0 to 1) | 1 (0 to 1) |
| Translation start site | 1 | 1 (0 to 1) | 0 (0 to 0) | 1 (1 to 1) |

TABLE 9
Enumeration of structural variants reconstructed from WGS of
CTCs discovered by tumor-normal matched analysis with the Structural
Variant detection workflow (Materials and Methods).

| Structural Variants | Total | Median (Range) |
|---|---|---|
| Total | 545 (100.0%) | 4 (1-72) |

By variant class

| | | |
|---|---|---|
| Deletion | 161 (29.5%) | 2 (0 to 23) |
| Inter-chromosomal translocation | 81(16.7%) | 2 (0 to 15) |
| Inversion | 84 (15.4%) | 2 (0 to 22) |
| Long range structural variant | 161 (29.5%) | 2 (0 to 34) |
| Tandem duplication | 48 (8.8%) | 1 (0 to 5) |

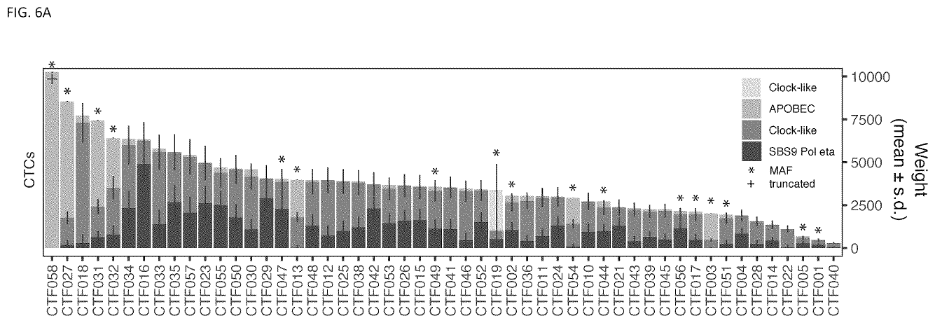

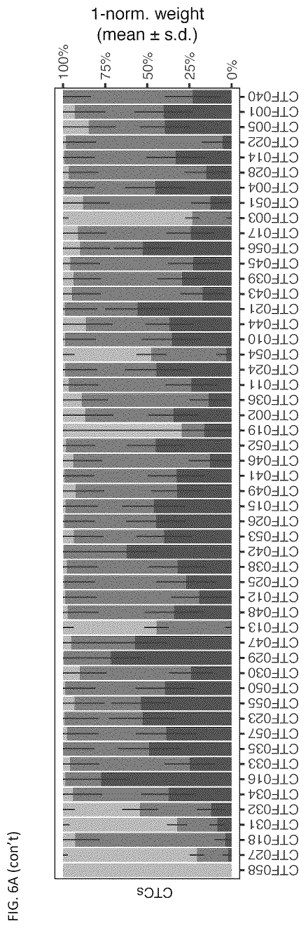

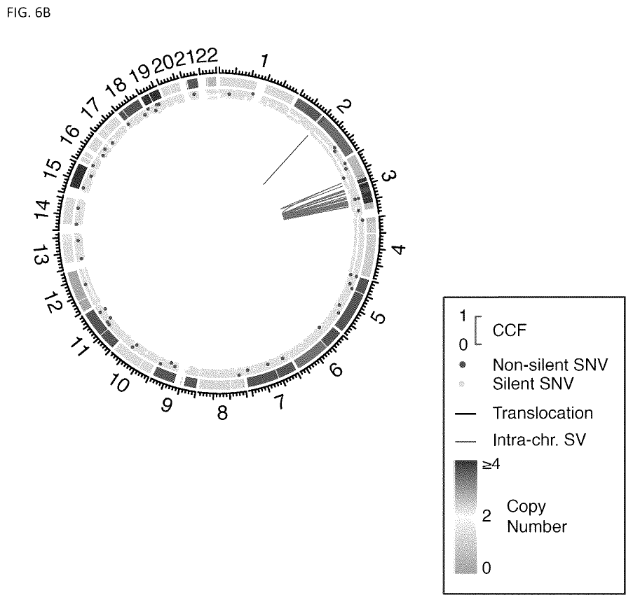

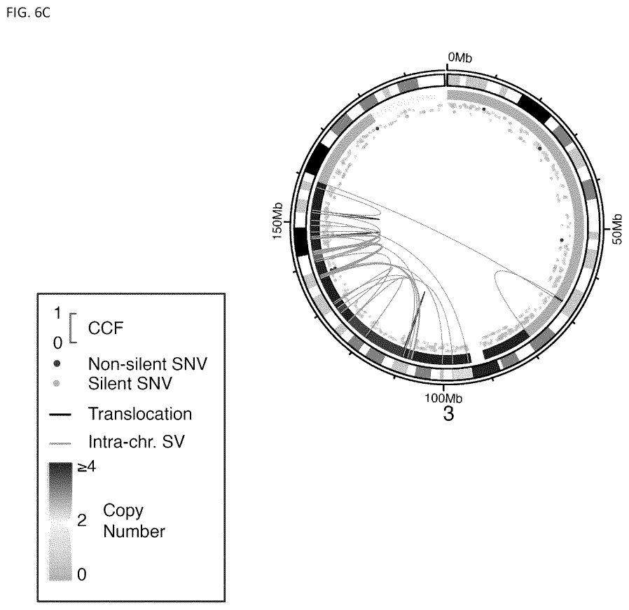

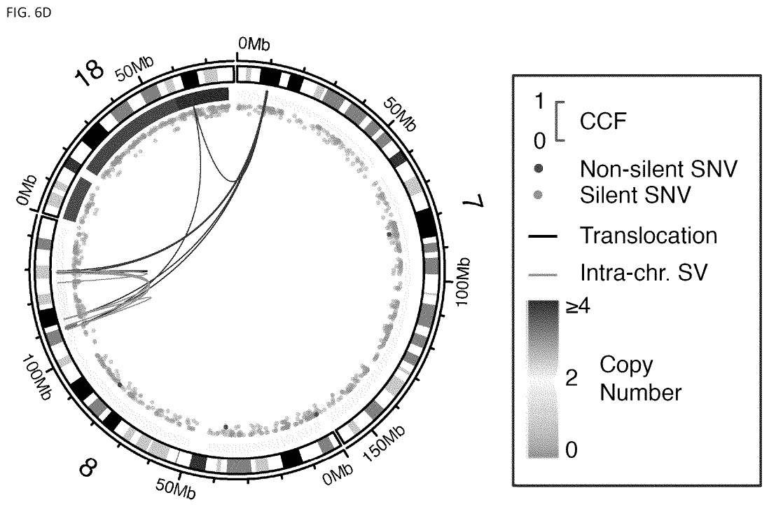

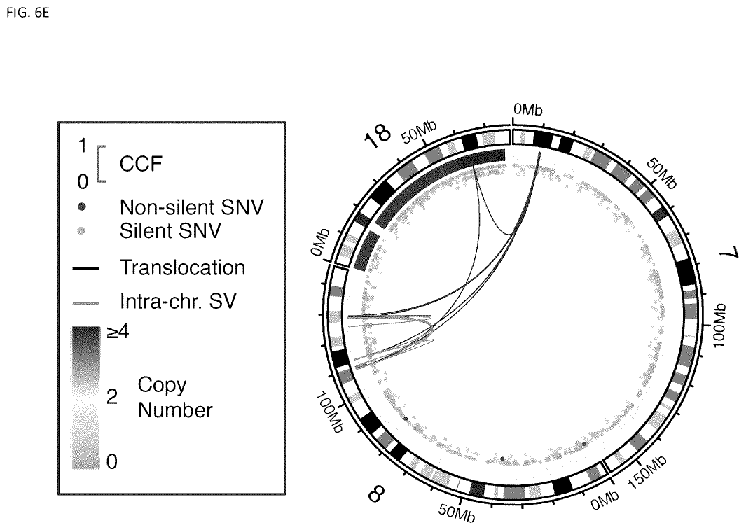

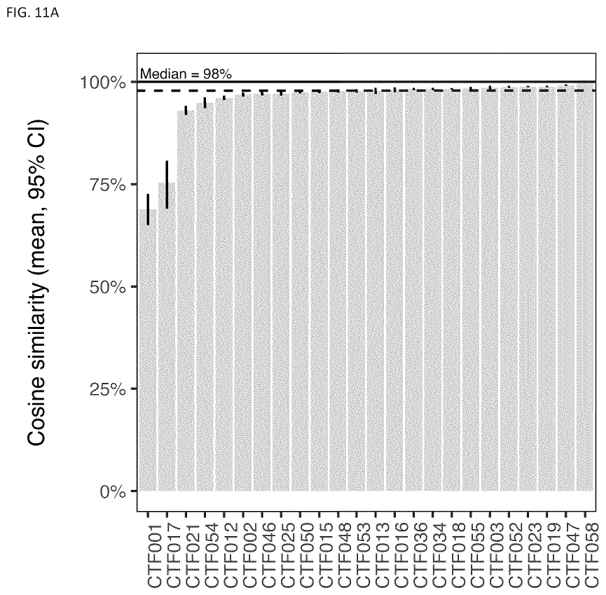

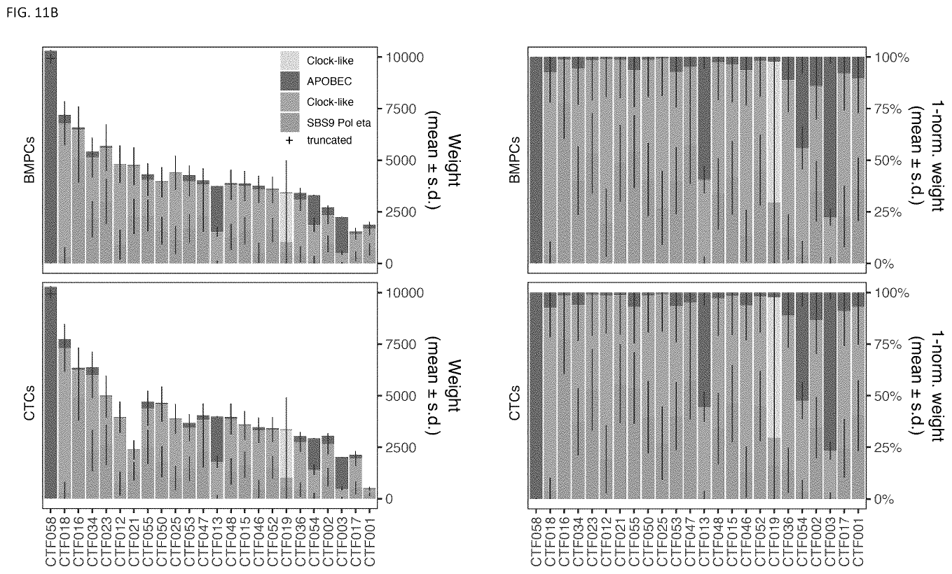

In addition to detection of somatic mutations, mutational processes and their distinctive signatures are an important feature in MM. Previous studies have revealed the contribution of AID, APOBEC and aging signatures to the mutational spectrum of MM (2,33,34). Recent studies highlighted that they may have clinical importance in SMM (35,36), with APOBEC shown to be enriched in those patients that progress or have a shorter time to progression. Mutation spectra and signature activity analysis reveals a comparable overlap of mutation types found between BM and CTCs (median cosine similarity 98%, IQR [94%, 99%]) [FIG. 11A]. Signatures extracted in a dataset comprised APOBEC, clock-like, and SBS9 as annotated by SignatureAnalyzer [FIG. 6A], with comparable contribution of mutational processes to the CTC and BM compartments [FIG. 11B]. In line with the literature, all participants with APOBEC contribution of more than 25% to the mutation spectrum were of MAF subtype (N=7/51, either IGH-MAF, MAFA, or MAFB). Finally, since WGS is not biased towards coding regions of the genome, more complex structural events could be deciphered, such as chromothripsis of chromosome 3 in CTF033 [FIGS. 6B and 6C] and chromoplexy of chromosomes 7, 8, and 18 involving the MYC locus in matched BM and PB of CTF034 [FIGS. 6D and 6E]. Overall, it was shown that additional novel information, not found by routine clinical tests, can be gained from CTCs that could be layered into patient assessment.

Here, proof was shown of principle that both enumeration and molecular analyses of CTCs using WGS have clinical utility in MM. MinimuMM-seq for comprehensive genomic characterization of CTCs was demonstrated, which robustly matches data derived from BM samples, and could be used as a surrogate to accompany and possibly replace BM biopsies for the diagnosis and longitudinal monitoring of MM patients through pathognomonic variant detection (translocations and hyperdiploidy) supplanting the need for clinical variables, or close observational monitoring of patients who are viewed as high-risk. The role of PB is well established in clinical workup of precursor MM with the assessment of markers such as monoclonal protein, serum albumin and light chain concentrations, however, does not take into consideration tumor biology analytes. Enriching and selecting CTCs from a large background of mononuclear cells, and subsequent extraction of nucleic acids from this minute cell fraction poses significant technical challenges, precluding their extensive molecular characterization. The above Examples provide the first demonstration that intact CTCs can be purified and used to detect complex structural events and chromosomal abnormalities which are hallmarks of MM disease. This was a substantial advance demonstrating that initiating events of MM can be detected upon genomic profiling of CTCs a priori, with similar sensitivity and specificity to clinical BM FISH. Importantly, this method can be the basis for clinical applications that use liquid biopsies to assay genomic biomarkers for diagnostic and prognostic purposes, where screening and monitoring of MM clones can be achieved using blood sampling only.

The prevalence of MGUS in the general population is estimated at 3-5% of individuals aged over 50 years, and SMM in 0.53% of individuals aged over 40 years old. Screening may be especially important for the SMM stage, which is heterogeneous and captures multiple clinical trajectories of disease including “MGUS-like”, indolent or “early MM-like” (Landgren, O, Hematology 2014, the American Society of Hematology Education Program Book 1, 194-204, 2017). Capturing high-risk patients, before end organ damage occurs, is of especially high importance for decisions on clinical management strategies. Using MinimuMM-seq, CTCs were observed to indeed represent the major BM clone upon blood sampling, with a high concordance of genomic profile between both compartments, illustrating that dominant clones in the BM possess the fitness to circulate and define the current tumors' biology. Initiating mutational profiles included primary translocations of t(11;14), t(4;14), t(14;16) and t(14;20). A similar representation of the overall clonal complexity in PB compared to BM was observed in matched sample patients. Notably, in few cases subclones with preferential circulation were observed in the PB compartment, harboring driver mutations such as KRAS conferring improved fitness. This shows that blood sampling and screening of CTCs is sufficient to reliably identify mutations of the BM to screen and monitor patients for initiating clones and potentially emerging high-risk clones that may have more aggressive characteristics. While genomic analyses of MM have not yielded targeted therapies to date (with the exception of venetoclax in t(11;14) patients), improved molecular profiling using CTCs and BM could indeed help to better stratify patients for clinical decisions using current models such as the four-risk factor 2/20/20 model (for SMM) (Mateos et al., Blood Cancer J 10, 102, 2020), Revised ISS(31) or mSMART models (for MM) (Mikhael et al., Mayo Clin Proc 88, 194-204, 2017). Targeting actionable mutations such as CDK, RAS/RAF and t(11;14) are currently being investigated within the MyDRUG trial (NCT03732703).

While BM remains the gold standard for diagnosis, recent studies have highlighted the importance of spatial heterogeneity in MM disease, a feature that is well-known from functional imaging. As such, current methods of BM biopsy at a single iliac crest or sternal site may not accurately represent the full picture of tumor biology, potentially missing high-risk genomic events of prognostic interest such as TP53 mutations (Rasche et al., Nat Commun 8, 268, 2017). The above Examples provide that high-risk arm-level abnormalities of 1q gain and deletion of chromosome 13 could be found at a higher cancer cell fraction in blood-derived CTCs than in BM biopsy samples of the same time point. Conversely, no high-risk events occurring exclusively in BM were detected. Furthermore, similar observations applied to driver mutations detected in KRAS and NRAS, which were all found to be of equal or higher clonality in the blood compared to BM. Thus, CTCs may assist in defining spatial genomic architecture and systemic disease through the detection of private mutations upon PB sampling not found in the standard BM biopsy site. This suggests that capturing CTCs could give a major unexpected advantage over BM biopsies, in that CTCs harboring such abnormalities lose dependence and diffuse into the blood from proliferative niches throughout the body before they settle and induce a clonal sweep and fixation at a primary or secondary regional site. Further studies of longitudinal abnormalities in matched BM and PB compartments will help elucidate this hypothesis.

MinimuMM-seq for CTCs also addresses the current challenge of repeated sampling of patients for the continuous monitoring of disease development and tumor evolution. CTCs at a single time point can provide assessment of genomic profile and actionable mutations, while longitudinal blood collection at multiple time points can track mutation architecture and clonal dynamics through CTCs, similar to that of the major BM clone, in a minimally invasive manner. In particular, it was shown that serial sampling of blood from untreated SMM patients can be used to identify the clonal complexity, phylogeny and evolution of CTCs, where emergence (of high-risk subclones with selective advantage) and extinction (of passenger subclones) is observed over time. Conceivably, this approach could also be applied in a treatment setting, to provide evidence for response or resistance, restaging and end-of-trial assessment where BM is unable to be collected, and measure how fitness landscape changes with treatment pressure. Indeed, in one SMM patient that began receiving early interventional therapy, it was shown that longitudinal liquid biopsies monitoring CTCs provide information on clonal dynamics and selection of high-risk subclones that emerge in real-time. This observation illustrated that dense sampling of blood for genomic profiling may assist to closely track patient response and tailor changes given a patient's specific evolving biology and clonal composition. As whole genome sequencing becomes less expensive, this approach will likely be easily accessible and may become more affordable than traditional FISH for clinical application. Many tests using next-generation sequencing, including germline whole genome sequencing, have been introduced in recent years, and this category will likely continue to expand. Furthermore, at MRD testing time points it was observed that CTCs are rarely present in the PB when patients were either MRD positive or MRD negative by NGS evaluation. This is similar to previous studies showing high false negativity of non-invasive MRD assessment by monitoring CTCs as a measure of MRD burden post-treatment compared to BM (Sanoja-Flores et al., Blood 134, 2218-2222, 2019).

Compared to higher burden SMM and overt MM, a minority of MGUS, lower-risk SMM, and MRD status patients may be eligible for genomic characterization by MinimuMM-seq at the first sampling date, however, monitoring by enumeration and moving to genomic characterization when CTC numbers increase could coincide with the time at which such an assay is relevant in the disease course. In particular, patients on treatment and being assessed for MRD may benefit from regular serial sampling of peripheral blood for CTC burden as an assessment of the duration of MRD status. The above Examples suggest copy number abnormalities face no lower limit of detection, while translocations could be detected in samples down to 50 CTCs and clonal mutations were reliably detected from around 300 CTCs. In the above Examples, researchers relied on affinity-based selection of PCs (CD138+38+), where, notably, CTCs could be missed due to potential dynamic surface protein expression or clonogenic potential of CD138− cells (Hosen et al., Leukemia 26, 2135-2141, 2012; Matsui et al., Blood 103, 2232-2236, 2004; and Paiva et al., Blood 122, 3591-3598, 2013). MinimuMM-seq provides a foundation for minimally invasive detection, enumeration and genomic interrogation of rare CTCs from the peripheral blood, illustrating the clinical potential of using liquid biopsies for monitoring and managing disease in MM. Addressing cost and strategies to bring WGS into the clinical standard of care will consequently provide an unbiased test to improve on and refine genomic biomarker assessment for patient diagnostics. Ultimately, CTCs possess great potential to enable precision oncology and prevention in MM and its precursor conditions.

The following methods were employed in the above examples.

### Patient Sample Collection

Blood samples from a cohort of 261 patients (84 MGUS, 155 SMM, 22 MM) were prospectively collected from the Dana-Farber Cancer Institute observational Precursor Crowd (PCROWD) study (NCT02269592) and the Plasma Cell Dyscrasias study. All patients provided written informed consent for the research use collection of peripheral blood and bone marrow samples (IRB #14-174 and #07-150). Research studies were carried out in accordance with the Declaration of Helsinki. Peripheral blood was drawn into CellRescue Preservative Tubes (Menarini Silicon Biosystems) and kept at room temperature before processing on the CellSearch instrument. Samples were all processed within 96 hours from time of collection. Patients with matched BM biopsies had routine clinical cytogenetics and FISH analysis performed at a molecular pathology laboratory.

### Enrichment and Enumeration of CTCs

From each patient, peripheral blood was collected in CellRescue Preservation Tubes (Menarini Silicon Biosystems) and processed on the The CellSearch® System (Menarini Silicon Biosystems) comprised of the CELLTRACKS® AUTOPREP® System and the CELLTRACKS ANALYZER II® using the CELLSEARCH Circulating Multiple Myeloma Cell (CMMC) Assay (Menarini Silicon Biosystems), for the enrichment and enumeration of circulating Multiple Myeloma cells. The sensitivity and linearity of circulating multiple myeloma cells using the CellSearch system has previously been reported (Foulk et al., Br J Heaematol 180, 71-81, 2018). Briefly, four milliliters of blood was removed from the CellRescue Preservation tube and processed on the CELLTRACKS® AUTOPREP® System using the CELLSEARCH Circulating Multiple Myeloma Cell (CMMC) Assay to enrich for myeloma cells following manufacturer's recommendations. Myeloma cells were immuno-magnetically captured using an anti CD138 antibody conjugated with ferrofluid. All captured cells were stained with an anti CD38 antibody conjugated with Phycoerythrin (PE), an anti CD19/CD45 antibody cocktail conjugated with Allophycocyanin (APC), to differentiate leukocytes from the circulating myeloma cells. All cell nuclei were stained with DAPI and then fixed with paraformaldehyde. The cartridges containing the enriched myeloma cells were placed in the CELLTRACKS ANALYZER II®, a semi-automated fluorescence microscope. The sample was then exported from CELLTRACKS ANALYZER II® and imported into The GateWorks software. The GateWorks software segmented the objects in the browser images, extracted object features and ‘gated’ those events that were most likely to be CMMCs for review by the user. The myeloma phenotype was CD138+/CD38+/DAPI+/CD19−/CD45−. Any background cells such as leukocytes, which had a phenotype CD138−/CD38−/DAPI+/CD19+/CD45+, were not counted as a myeloma cell. Samples were then removed from the cartridges and stored in glycerol stocks at −20 °C for subsequent downstream molecular characterization.

### Sorting of CTCs

Upon enrichment of peripheral blood samples using The CellSearch® System, ~100-10,000 leukocytes may remain in the background of the extracted sample, therefore methods of cell sorting were employed for sorting of pure CTCs to obtain high tumor fraction pools of CTCs. High sensitivity fluorescence-activated cell sorting (FACS) (BD Biosciences Aria II) was used to sort pure populations of tumor PCs and germline WBCs based on immunophenotypic gating of CD138+38+45− and CD45+CD138−CD38−, respectively.

### DNA Library Construction

Sorted samples underwent DNA purification (Thermo Fisher PicoPure DNA Isolation Kit) and library preparation using the NEBNext Ultra II FS DNA Library Prep kit (New England Biolabs) with unique dual index adapters (NEBNext Multiplex Oligos) according to manufacturers' instructions. Final library fragment sizes were assessed using the BioAnalyzer 2100 (Agilent Technologies), with yields being quantified by Qubit 3.0 fluorometer (Thermo Fisher Scientific) and qPCR (KAPA Library Quantification Kit).

### DNA Sequencing and Genomic Data Analysis

Final sample libraries were normalized and pooled, before whole genome sequencing was performed on Illumina Novaseq6000 S4 flowcells, 300 cycles paired-end reads, at the Genomics Platform of the Broad Institute of MIT and Harvard. Whole-Genome Sequencing analysis was performed on an in-house cloud-based HPC system for copy number, mutation, and structural variant analysis and detailed steps are described herein. Briefly, sequencing reads were aligned to the hg19 reference genome with the bwa mem v0.7.7 algorithm, and duplicate reads were marked with MarkDuplicates from picard v1.457, indels were realigned with GATK 3.4 IndelRealigner, and base qualities were recalibrated with the GATK 3.4 BaseRecalibrator software. Mutations were called with MuTect1 (Cibulskis et al., Nat Biotechnol 31, 213-219, 2018) and small indels with Strelka2 (Kim et al., Nat Methods 15, 591-594, 2018), (for single nucleotide variants and indels), and were filtered (1) against a panel of normals (PoN), (2) for potential technical artefacts (oxoG) and (3) for multiple alignment with BLAT. After copy number normalization with AllelicCapSeg, ABSOLUTE (Carter et al., Nat Biotechnol 30, 413-421, 2012) solutions were manually reviewed to estimate mutations cancer cell fraction (CCF), purity and ploidy of tumor samples. Phylogeny reconstruction was performed with the PhylogicNDT (Leshchiner et al., bioRxiv, 508127, 2019) suite of tools with the following parameters: minimum cancer cell fraction: 20%, minimum coverage: 10, number of iterations: 1,000. Mutational signatures were quantified with SignatureAnalyzer (Kim et al., Nat Genet 48, 600-606, 2016; Kasar et al., Nat Commun 6, 8866, 2015) method and n=100 runs. Structural variants were detected and filtered as previously described (Morton et al., Science 372, 6543, 2021.

### Statistical Analyses

Quantitative bio-clinical variables were described with median and interquartile range (IQR) or absolute range, or with mean and standard deviation. Average difference between groups was assessed with Kruskal-Wallis method for multiple group testing followed by Dunn's post hoc tests, and/or Wilcoxon test for 2 groups. When the mean is estimated from a random process, it is given with its 95% confidence interval. Qualitative variables were described using the frequency of their respective modalities. Distinct distribution between groups was assessed with χ\hat{ }2 Pearson's test (or Fisher's exact test if appropriate). Patients were stratified for progression-free survival by presence of CTCs and by quartiles of CTC enumeration. Time to event was calculated from screening to clinical progression to multiple myeloma, or death of any cause, whichever occurred first. Likelihood ratio test statistic is reported in the absence of an event in the non-progressor group. P values were corrected for multiple testing with the Benjamini-Hochberg method. Adjusted p values under 0.05 were considered significant. All calculations were done using R 4.1.1 software.

### Data Availability

Data and code used in the analysis are available at https://github.com/jalberge/ms-cmmcs/ or in the Supplementary Materials and Methods. The sequencing data presented in the current publication have been deposited in and are available from the dbGAP database under accession number phs003084.v1.

### Fish Analysis

FISH analysis was performed on BM aspirate cells with fluorescence in situ pretreatment, hybridization and fluorescence microscopy in accordance with laboratory specimen-specific protocols. Fifty plasma cell (PC) nuclei were analyzed per probe set, as available when at least 15 PC were available. Otherwise, analysis was considered insufficient. FISH analysis was performed by two qualified clinical cytogenetic technologists and interpreted by a board-certified (American Board of Medical Genetics and Genomics) clinical cytogeneticist. BM aspirate samples were subjected to one of two MM FISH panels. A limited panel included probes designed to detect high risk multiple myeloma abnormalities; loss of chromosome 17p (TP53 [17p13.1]/D17Z1 [CEN17], Abbott Molecular, Des Plaines, IL), gain or amplifications of chromosome 1q (TP73 [1p36.3]/1q22 [1q22], laboratory-developed test) and IGH gene rearrangements (break-apart probe (BAP), laboratory-developed test, and a IGH::CCND1 dual color dual fusion (DF) probe set for t(11;14) (Abbott Molecular). If the IGH break-apart probe was abnormal (separation of 5′ and 3′ IGH probe sets; i.e. 1R1G1F, 1R1F or 1G1F) without evidence of IGH::CCND1 fusion, double fusion (DF) probe sets to identify classic partners t(4;14) (IGH::NSD2 or FGFR3, Abbott Molecular), t(14;16) (IGH::MAF, Abbott Molecular), t(14;20) (IGH::MAFB, laboratory-developed test) were subsequently performed. In addition to the probes comprised on the limited panel, the extended panel also included probes to detect loss of chromosome 13/13q (RB1 [13q14]/LAMP1 [13q34]/D4Z1 [CEN4], Abbott Molecular) and MYC rearrangements (BAP, Abbott Molecular). Reflex analyses for the extended panel included those from the initial panel in addition to a DF probe to identify t(6;14) (IGH::CCND3, laboratory-developed test) in the setting of an IGH rearrangement. If ploidy status could not be determined by flow cytometry, investigation for gains of chromosomes 9, 15 (D9Z1 [CEN9]/D15Z4 [CEN15], Abbott Molecular), 3 and 7 (D3Z1 [CEN3]/D7Z1 [CEN7], Abbott Molecular) was also sought. In addition, a smaller panel including FISH probes to detect classic multiple myeloma progression markers; gain or amplification of chromosome 1q, loss of TP53 at chromosome 17p13 and MYC rearrangements. The level of detection required to identify abnormalities was as follows: a minimum of 3 cells displaying fusion signals in the setting of DF probes, a minimum 5 cells with disrupted or separated signals in the setting of BAP probes and a minimum 5 cells with tetraploidy for tetraploid clones or 10 supporting cells for enumeration probes.

Cytoplasmic Immunoglobulin In Situ Hybridization (cIg-FISH)

Pre-analysis to assess adequacy of PC content of samples prior to cIg-FISH was performed using flow cytometry. Samples with more than 0.1% PC (identified with anti-CD19-PerCP 5.5 (clone SJ25C1, BD Biosciences), anti-CD38-APC (clone REA671, Miltenyi Biotec), anti-CD138-BV421 (clone MI15, BD Biosciences), anti-CD45-BB515 (clone HI30, BD Biosciences), anti-cytoplasmic kappa and lambda) were deemed satisfactory for cIg-FISH analysis. After hybridization of slides and post-hybridization wash steps, slides were washed with PBS and left to air dry. PCs were stained with fluorescein isothiocyanate (FIT-C)-conjugated antibodies directed against the kappa and lambda light chains. Only light-chain positive cells were targeted for scoring during FISH analysis. Samples processed before 2020 underwent cIg-FISH-based PC enrichment.

### Fluorescence Activated Cell Sorting (FACS-FISH)

BM cells (approximately 20×106) were lysed in ACK lysis buffer for 5 minutes, followed by PBS wash ×2 (lyse-wash procedure). The cell pellet was re-suspended in 3% BSA/PBS. Next, 10×106 cells were incubated for 15 minutes with the following antibodies: anti-CD19-PerCP 5.5 (clone SJ25C1, BD Biosciences), anti-CD38-APC (clone REA671, Miltenyi Biotec), anti-CD45-BB515 (clone HI30, BD Biosciences), anti-CD56-PE-Cy7 (clone NCAM16.2, BD Biosciences), anti-CD138-BV421 (clone MI15, BD Biosciences), and anti-CD319-PE (clone REA150, Miltenyi Biotec). The specimen was centrifuged and re-suspended in 1.5 mL of PBS. Sorting was performed on BD FACSMelody cell sorter (BD Biosciences, San Jose, CA). Sorting streams were defined for each case separately, using gates to include CD138-positive, CD319-positive, CD38-bright, CD56-positive and/or CD45-negative plasma cells, and separate them from normal plasma cells. A purity of at least 95% was achieved and verified by Kaluza software (Beckman Coulter Life Sciences, Indianapolis, IN). A minimum of 1000 sorted PCs collected in methanol/acetic acid was required to carry out FISH analysis. The sorted specimen was then processed for FISH analysis. Samples processed between 2020 and 2022 underwent PC enrichment via FACS (FACS-FISH).

### DNA Library Construction for Preliminary Ultra Low-Pass Whole-Genome Sequencing

Minipools of CTCs that were sorted by DEPArray underwent whole genome amplification using the Ampli1 kit (Menarini Silicon Biosystems), followed by PCR-free library preparation with unique dual indices (KAPA HyperPrep kit and KAPA Unique Dual Indexed Adapter kit), library quantification and ultra low pass whole genome sequencing (ULP-WGS) on RapidRun flowcell of HiSeq2500 (Illumina). ULP-WGS was used for molecular assessment to detect hyperdiploidy and copy changes as genomic biomarker events of MM disease, with ichorCNA (Adalsteinsson et al., 2017) analyses performed to determine copy number variant (CNV) events and infer tumor fraction.

### Genomic Sequence Alignment and Processing

Sequencing reads were aligned to the hg19 reference genome with the bwa mem v0.7.7 algorithm (Li, 2013) and the -M option. Duplicates were marked with the MarkDuplicates function from picard tools v1.475. BAM files were then processed for indel realignment with the RealignerTargetCreator (parameters -dcov 250 -nt 1 -L 9) and IndelRealigner functions and for base calling quality recalibration with the BaseRecalibrator function of GATK 3.4 and with the Broad institute's b37 bundle reference dbSNP 138, known indels, and variantEvalGoldStandard. Samples were checked for absence of contamination and sample mismatch with the CrossCheckLaneFingerprints function from picard v1.475 and with the ContEst tool (Cibulskis et al., 2011).

### Panel of Normal

Paired germline sequencing data were used as a panel of normals (PoN) to normalize and control for artifacts and variability of unknown source in copy number profiling and false-positive mutations. The copy number PoN was generated with the ReCapSeg algorithm (Lichtenstein et al., AACR, 2016). The token file for point mutations was generated with the CGA_Token_PoN_Maker v0.1 Firecloud task (available here: https://portal.firecloud.org/?return=terra#methods/getzlab/CGA_Token_PoN_Maker_v0.1_Jan2019/2 with a free account) and run on Terra.

### Copy Number Analysis

Copy number profiling was performed with the AllelicCapSeg algorithm (Landau et al., Cell 152, 714-726, 2013). AllelicCapSeg uses CNV and SNP haplotyping to infer allelic local copy number. The GATK CNV task was used to calculate segment copy number ratio between tumor and normal. CNV values within [−0.1, 0.1] in the log 2 ratio space were considered normal. Results were normalized with the PoN built from matched-germline samples of this cohort processed and sequenced with the same protocol. The heterozygotes sites were obtained from MuTect1 call_stats results, and used as an input of the AllelicCapSeg algorithm. Copy number estimate and minor allele frequencies were reported per tumor-normal pair and used in mutation calling step to estimate cancer cell fraction (CCF) of mutations with ABSOLUTE (Carter et al., Nat Biotechnol 30, 413-421, 2012).

### Structural Variant Detection

Three algorithms were used to detect and filter structural variants (SVs) genome-wide similar to Morton, Karyadi, Stewart and colleagues (Morton et al., Science 372, 6543, 2021). Briefly, Manta (Chen et al., Bioinformatics 32, 1220-1222, 2016), dRanger, and SVaBa (Wala et al., Genome Res 28, 581-591, 2018) algorithms are executed in parallel on paired tumor-normal BAM files. dRanger was run with the following parameters: tminmapq=5, minpairs=2, windowsize=2000, nminwindow=2000, minsomratio=50, nminspanfrac=0.5, minscoreforbp=0.01. Manta and SVaBa were run with the default parameters and the following filters were applied to each output: minscoreforbp=0.1, min_span=200, max_pon=1, max_norm=2, min_tum_SR=0 (1 for Manta), min_tum_RP=1 (0 for Manta), min_tum=4. Specific capture of reads supporting immunoglobulin (IG) translocations restricted to regions of interest was assessed with parameters min_tum=2 and minimum number of split-read and read-pairs both set to 0. Regions of interest were pre-defined as genomic intervals encompassing any IGH translocation from the CoMMpass study release IA15 and found in 1% of participants. Next, BreakPointer (Drier et al., Genome Res 23, 228-235, 2013) was used to aggregate results from the SV detection algorithms and to score structural variants after local assembly with the Smith-Waterman algorithm. SVs were all manually reviewed in IGV.

### Mutation Calling

Mutations and short indels were detected with MuTect1 and Strelka2 respectively. MuTect1 (Cibulskis et al., Bioinformatics 27, 2601-2602, 2013) was used in matched tumor-normal pairs genome-wide. To estimate the power to detect somatic variants given purity, ploidy, and cancer cell fraction, the MuTect formula was used. Additionally, Strelka2 (Kim et al., Nat Methods 15, 591-594, 2018) was used to characterize short insertions and deletions (indels). The DeTiN algorithm (Taylor-Weiner et al., Nat Methods 15, 531-534, 2018) was used to estimate tumor-in-normal contamination and to rescue somatic mutations originally discarded by MuTect1 and Strelka2. Filtering of mutations was done with in-house code and included detection and filtering of oxoG artefacts (Costello et al., Nucleic Acids Research 41, e67-e67, 2013) detection of mutation in the panel of normals. Additionally, mutations were inspected with the BLAT algorithm and for each sequencing read supporting a somatic mutation, the alternative alignments suggested by BLAT are examined. Mutations that are only supported by reads which are ambiguously mapped are removed. Finally, SNVs and indels were annotated with GATK's Funcotator v1.6 and used as an input of the ABSOLUTE algorithm (Carter et al., Nat Biotechnol 30, 413-421, 2012).

### Absolute Copy Number, Mutations, and Phylogeny Trees

ABSOLUTE (Carter et al., Nat Biotechnol 30, 413-421, 2012) was used to estimate purity, ploidy, and subclonal composition of mutations and copy number abnormalities. ABSOLUTE solutions were all reviewed manually and chosen based on optimal fit of subclonal SNV multiplicity and fraction of alternate reads and when available, BM samples were cross-validated with the fraction of cells bearing arm-level abnormalities according to matched FISH reports. For participants with matched BMPCs and CTCs available, and for the participant with serial sampling over time, the union of mutations between both compartments (or between both timepoints) was additionally force-called with the forcecaller task (available with a free Terra account at the following location: https://portal.firecloud.org/?return=terra#methods/danielr/forcecall_snps_and_indels/7). Cancer cell fraction from the union of mutations was then calculated again with ABSOLUTE. Phylogeny trees between both compartments (or between timepoints) were reconstructed with the PhylogicNDT algorithm (Leshchiner et al., bioRxiv, 508127, 2019). PhylogicNDT was run with the following parameters: minimum cancer cell fraction: 20%, minimum coverage: 10, number of iterations: 1,000.

### Mutational Signature Analysis

Mutational processes were weighted with the ARD-NMF decomposition provided in the SignatureAnalyzer method (Kasar et al., Nat Commun 6, 8866, 2015; Kim et al., Nat Genet 48, 600-606, 2016; Taylor-Weiner et al., Genome Biol 20, 228, 2019). SignatureAnalyzer was run with the following parameters: reference=pcawg_COMPOSITE, objective=poisson, n=100. The PCAWG composite reference dataset is used to annotate single-base substitutions in their pentanucleotide neighborhood (SBS with 1536 context possibilities), indels, and double-based substitutions, and assigned to most likely reference signature based on cosine similarity metrics. Stability of NMF decomposition was assessed with aggregation of signatures weights across all ARD-NMF runs. For matched BMPCs and CTCs sequencing data, bootstrapping (N=1000) was used to estimate mean and 95% confidence intervals of the mutations cosine similarity between assays.

### BCR Alignment

BCR sequences were reconstructed with the mixcr (Bolotin et al., Nat Biotechnol 35, 908-911, 2017; Bolotin et al., Nat Methods 12, 380-381, 2015) set of algorithms in “shotgun analyze” mode with default parameters for DNA BCR sequence reconstruction and with the “--only-productive” flag. Input regions included sequence mapping to reference immunoglobulin heavy chain loci hg19 coordinates. BCR hits were then compared between BM and PB and allele frequencies in both compartments were systematically reported.

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-51. (canceled)

52. A method for treating a subject for a hematological malignancy, comprising:
(a) performing whole genome sequencing of nucleic acids obtained or derived from circulating tumor cells isolated from a biological sample of said subject, thereby generating sequencing data, wherein said subject has been administered an agent for said treatment of said hematological malignancy;
(b) identifying a multiple myeloma (MM) stage of said subject based at least in part on said sequencing data; and
(c) treating said subject with said agent based at least in part on said MM stage.

53. The method of claim 52, wherein said agent comprises a chemotherapeutic agent, an immunotherapy agent, or a combination thereof.

54. The method of claim 52, wherein said identifying said MM stage comprises detecting one or more genomic alterations comprising an aneuploidy, a translocation, a chromosomal gain, a chromosomal deletion, a driver mutation, or any combination thereof.

55. The method of claim 52, further comprising sorting said circulating tumor cells based upon an immunophenotype of CD19−, CD138+, CD38+, and CD45−.

56. The method of claim 52, wherein said whole genome sequencing does not comprise whole-genome amplification.

57. The method of claim 52, further comprising sorting said circulating tumor cells based upon an immunophenotype CD19−, CD138+, CD38+, CD45−, or any combination thereof.

58. The method of claim 52, wherein said circulating tumor cells are isolated from said biological sample using an immunofluorescence-based technique.

59. The method of claim 52, wherein said hematological malignancy is multiple myeloma (MM), Monoclonal Gammopathy of Undetermined Significance (MGUS), or Smoldering Multiple Myeloma (SMM).

60. The method of claim 52, wherein said method is performed on a plurality of biological samples obtained from said subject at different time points, and wherein said identifying said MM stage is used to monitor clonal evolution of said hematological malignancy or to assess an effectiveness of said agent in said subject over time.

61. A method for identifying resistance to an agent administered to a subject for treatment of a hematological malignancy, comprising:
(a) performing whole genome sequencing of nucleic acids obtained or derived from circulating tumor cells isolated from a biological sample of said subject, thereby generating sequencing data, wherein prior to said whole genome sequencing, said subject has been administered said agent for said treatment of said hematological malignancy; and
(b) identifying said resistance to said agent administered to said subjects for said treatment of said hematological malignancy based at least in part on said sequencing data.

62. The method of claim 61, comprising treating said subject with said agent based at least in part on said resistance to said agent.

63. The method of claim 62, wherein said agent comprises a chemotherapeutic agent, an immunotherapy agent, or a combination thereof.

64. The method of claim 61, wherein said identifying said resistance comprises detecting a genomic alteration comprising of an aneuploidy, a translocation, a chromosomal gain, a chromosomal deletion, a driver mutation, or any combination thereof.

65. The method of claim 61, wherein said whole genome sequencing does not comprise whole-genome amplification.

66. The method of claim 61, further comprising sorting said circulating tumor cells sorted based upon an immunophenotype CD138+, CD19−, CD45−, CD38+, or any combination thereof.

67. The method of claim 61, wherein said circulating tumor cells are isolated from said biological sample using an immunofluorescence-based technique.

68. The method of claim 61, wherein said hematological malignancy is multiple myeloma (MM), Monoclonal Gammopathy of Undetermined Significance (MGUS), or Smoldering Multiple Myeloma (SMM).

69. The method of claim 61, further comprising sorting said circulating tumor cells based upon an immunophenotype of CD19−, CD138+, CD38+, and CD45−.

70. The method of claim 61, wherein said identifying said resistance comprises detecting: a translocation selected from the group consisting of t(11;14), t(14;16), t(4;14), t(6;14), t(14;20), t(8;14), t(2;8), and t(8;22); a chromosomal gain comprising a 1q gain; a chromosomal deletion comprising a 1p deletion; a chromosome 13q deletion; a chromosome 16q deletion; a chromosome 17p deletion; a driver mutation comprising a non-silent mutation to a KRAS and/or NRAS gene sequence; a mutation to a DIS3, FAM46C, BRAF, TP53, or any combination thereof gene sequence; or any combination thereof.

71. The method of claim 61, wherein said method is performed on a plurality of biological samples obtained from said subject at different time points, and wherein said identifying said resistance is used to monitor clonal evolution of said hematological malignancy or to assess an effectiveness of said agent in said subject over time.
